(19) United States
(12) Patent Application Publication
(10) Pub. No.: US 2009/0186949 A1
(43) Pub. Date: Jul. 23, 2009

(54) Method for Treating Endothelial and Epithelial Cell Disorders by Administering High Molecular Weight PEG-Like Compounds

(75) Inventors: John C. Alverdy, Glenview, IL (US); Joe G.N. Garcia, Chicago, IL (US); Steven Dudek, Frankfort, IL (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: THE UNIVERSITY OF CHICAGO, Chicago, IL (US)

(21) Appl. No.: 12/281,102

(22) PCT Filed: Feb. 28, 2007
(86) PCT No.: PCT/US2007/063007

§ 371 Date: Nov. 14, 2008

Related U.S. Application Data
(60) Provisional application No. 60/778,165, filed on Feb. 28, 2006.
(60) Provisional application No. 60/787,548, filed on Mar. 29, 2006.

(51) Int. Cl. A61K 31/08; A61P 11/00

(52) U.S. Cl. 514/723

(57) ABSTRACT

The invention provides methods of screening for modulators of microbial PA-I lectin/adhesin activity, including modulators of PA-I expression, as well as the modulators so identified, pharmaceutical compositions and kits containing such modulators. These modulators include soluble and membrane-bound bacterial signaling compounds produced by cells of a host containing a microbial pathogen. Methods for preventing and treating cell disorders, such as epithelial cell disorders including gut-derived sepsis, a burn injury, neonatal necrotizing enterocolitis, severe neutropenia, toxic colitis, inflammatory bowel disease, enteropathy, transplant rejection, pouchitis, pig belly, Pseudomonas-mediated opthalmologic infection, Pseudomonas-mediated otologic infection and Pseudomonas-mediated cutaneous infection, using the modulators are contemplated, as are methods for ameliorating a symptom associated with such a disorder. The invention further provides methods of treating or preventing disorders of the lung, including disorders affecting endothelial or epithelial cell barrier function, such as acute respiratory distress syndrome or pulmonary edema, as well as methods of preventing such disorders, and the above-named disorders, such as neonatal necrotizing enterocolitis, by administering a high molecular weight polyethylene glycol-like compound. Related methods of ameliorating a symptom associated with one of these disorders is also contemplated.

The invention was made with U.S. Government support under contract nos. HL058064 and GM062344. The U.S. Government has certain rights to this invention.

### FIELD OF THE INVENTION

The invention generally relates to the field of prophylactic and therapeutic modulation of microbial pathogen involvement in disorders and diseases of mammals such as humans. The invention also relates specifically to the field of prophylactive and therapeutic modulation of disorders of lung cell barrier function.

### BACKGROUND

Acute lung injury (ALI) and acute respiratory distress syndrome (ARDS) are inflammatory lung syndromes characterized by diffuse alveolar infiltration, hypoxemia, respiratory failure, and deaths due to multi-organ failure. Mortality rates in ARDS, the most severe ALI clinical scenario, range from 34 to 58% (MacCallum et al., Curr Opin Crit Care 11:43-49, 2005) with 150,000-200,000 ALI cases per year in the United States (Ware et al., N Engl J Med 342:1334-1349, 2000) and an incidence of 17-34 cases/100,000 people per year in Europe, Australia, and other developed countries. Thus ALI and ARDS constitute a major healthcare burden due to the intensive and often prolonged intensive care unit (ICU) hospitalizations. In addition to these epidemiologic studies, race and gender differences in ARDS deaths in the United States over the past several decades have demonstrated an increase in incidence and mortality due to sepsis and ALI in African-Americans when compared with Caucasians (Moss et al., Crit Care Med 30:1679-1685, 2002). ALI is usually caused by sepsis, acid inhalation, or trauma with mechanical ventilation, an intervention strategy commonly used in the ICU to treat ALI, potentially exacerbating ALI pathophysiology and reducing survival if excessive (Slutsky et al., Chest 116:9 S-15S, 1999, Tremblay et al., Crit Care Med 30:1693-1700, 2002, Kamat et al., Alcohol Clin Exp Res 29:1457-1465, 2005). The hallmarks for ALI, i.e., cellular and spatial heterogeneity, profound high permeability, leukocyte influx, and lung edema, are often augmented by mechanical ventilation in animal models of ALI (Zhang et al., Anesthesiology 97:1426-1433, 2002) and are contributing factors for death due to multi-organ failure (Slutsky et al., Am J Respir Crit Care Med 157:1721-1725, 1998).

Despite recent advances in low tidal volume mechanical ventilation and a better understanding of the underlying inflammatory pathophysiology of ALI, there remain few effective treatments for this devastating illness. ALI and the more severe acute respiratory distress syndrome (ARDS) represent a spectrum of common syndromes in response to a variety of infectious and noninfectious insults. The immune response to insults, such as infection or shock, includes recruitment of neutrophils and other inflammatory cells, induction of proinflammatory cytokines, and subsequent generation of reactive oxygen intermediates, which cause tissue damage and contribute to the induction and perpetuation of ALI (Lang et al. Chest 122:314 S-320S, 2002, 3). One consequence of enhanced oxidative stress is the peroxidation of phospholipids, abundantly present in the surfactant layer, into active metabolites, which have been observed in a variety of acute and chronic inflammatory diseases of the lung, including ARDS and asthma (Chabot et al., Eur Respir J 11:745-757, 1998, Wood et al., Eur Respir J 21:177-186, 2003). In addition, reactive nitrogen species generated during acute inflammation may also modify blood-borne and cellular lipids, representing a novel source of bioactive oxidized or nitrated phospholipids (Pennathur et al., Biol Chem 279:42977-42983, 2004, Kalyanaraman, Proc Natl Acad Sci (USA) 101:11527-11528, 2004). Increased production of exhaled isoprostanes serves as an index of oxidant stress and lipid oxidation in a number of lung pathologies (see Morrow et al., Am J Respir Crit Care Med 166:S25-S30, 2002, for review) and is elevated in ALI (Carpenter et al., Chest 114:1653-1659, 1998). After tissue insult, oxidized phospholipids released from membrane vesicles may serve as stress signals, triggering both pro- and anti-inflammatory cascades.

The infectious insults giving rise to lung injury and disease are frequently attributable to pathogenic microbes, including opportunistic bacterial pathogens, which pose a significant health threat to man and a variety of domesticated and wild animals. One exemplary class of organisms is the Pseudomonads, which are opportunistic pathogens. Pseudomonas aeruginosa, a prototypical member of the Pseudomonads, is a ubiquitous bacterium found in soil, water, and plants and can be part of the normal flora in humans. In the context of human infection, it is an opportunist which causes disease primarily in patients with other underlying disease states. During physiologic stress, as occurs during critical illness or severe inflammation, the presence of P. aeruginosa in the lung or intestine of patients is associated with a poor prognosis and can cause lethal pneumonia or gut-derived sepsis. P. aeruginosa is the most common gram-negative bacterium isolated among cases of nosocomial infection and carries the highest reported fatality rate of all hospital acquired infections. The mere presence of this pathogen within the intestinal tract of a critically ill patient is associated with a four fold increase in mortality, independent of its dissemination to remote organs. It is generally reasoned that the observed increase in mortality of critically ill patients colonized by powerful nosocomial pathogens is simply due to lowered host resistance. Yet an alternative explanation for the continued high mortality rate among such severely stressed patients, despite more powerful antibiotics, is that certain opportunistic pathogens have evolved highly sophisticated mechanisms to sense both the susceptibility and liability of a physiologically stressed host and respond with enhanced virulence.

Despite improvements in antibiotics and patient care, mortality rates from severe sepsis following surgical injury and infection have remained unchanged for decades. Among patients at risk for severe sepsis, it has become axiomatic that those subjected to the greatest degree and number of physiologically stressful insults (termed “hits”), suffer the greatest mortality. While advances in medical care can rescue patients from many of these physiologic “hits”, the incidence and mortality from sepsis has been held constant by the survival of patients subjected to unprecedented levels of catabolic stress who become subsequently colonized by some of the most virulent and resistant nosocomial pathogens known to man. The intestinal tract reservoir is the anatomic site on which physiologic stress exerts a profound effect on organ function, and in which the greatest microbial burden accumulates through the course of such stress. That intestinal pathogens acquired during the course of critical illness might play a major role in the mortality due to sepsis has led to the term gut-derived sepsis. Since as many as 30% of critically ill patients with severe sepsis have no identifiable pathogen or focus of infection, the intestinal microflora could play a major role in driving and sustaining the systemic inflammatory response.

A recent surveillance study of 35,790 non-duplicate, gram-negative aerobic bacteria isolated from intensive care units (ICUs) in 43 US states identified P. aeruginosa as the most common gram-negative species isolated among critically ill patients (Neuhauser et al., JAMA, 289:885-8, 2003). The mere presence of P. aeruginosa in the intestinal tract of a critically ill host is associated with a 4-fold increase in mortality. Antibiotic resistance to this pathogen has increased 70% over the last 5 years and crude mortality statistics demonstrate that P. aeruginosa continues to be associated with the highest case fatality rate of all nosocomially acquired infections. Numerous studies have now confirmed that the intestinal tract is the single most important reservoir for subsequent infection with this pathogen (Arbo et al., Eur J Clin Microbiol Infect Dis, 17:727-30, 1998). A direct role for intestinal P. aeruginosa in the mortality of critically ill patients is supported by a recent prospective randomized trial of approximately 800 critically ill patients, half of whom where treated with non-absorbable oral antibiotics to decontaminate the intestinal tract of its flora. A statistically significant reduction in mortality (about 17%) was observed in antibiotic decontaminated patients in association with a significant reduction in antibiotic resistant strains of P. aeruginosa isolated from feces. Yet precisely how intestinal P. aeruginosa contributed to the mortality of these critically ill patients remains unknown.

Microbial pathogens such as P. aeruginosa can express a protein termed PA-I lectin/adhesin (PA-I). PA-I is a lectin, comprised of four 13 kDa subunits, that binds to D-galactose and its derivatives. Among monosaccharides, the binding specificity is strictly for galactose, with the exception of N-acetyl-D-galactosamine (GalNAc). Disaccharides containing a terminal α-D-galactose residue bind PA-I. The highest binding affinity to date has been demonstrated for the disaccharide mellibiose (α-GAl1-6GlC). P. aeruginosa strains suspended in a 0.6% solution of the specific PA-I binding sugar GalNAc or the disaccharide mellibiose, have markedly decreased adhesion to intestinal epithelial cells and do not alter their barrier function. The distribution of PA-I in bacteria can be either primarily intracytoplasmic or extracellular, depending on its environment. When bacteria are grown in ideal growth conditions, about 85% of PA-I is located intracellularly with small, but significant, amounts located within the cytoplasmic membrane, on the outer membrane, and in the periplasmic space. In sharp contrast, within the intestinal tract of a stressed host, PA-I abundance is increased and localizes to the outer membrane facilitating the adherence of P. aeruginosa to the intestinal epithelium. In addition, there is evidence that free PA-I is shed into the extracellular milieu and can be detected at concentrations as high as 25 μg/ml in both culture supernatants and sputum from P. aeruginosa infected lungs. This finding is of considerable importance, as treatment of cultured epithelial cells (e.g., T-84, Caco-2bbe, MDCK, airway epithelial cells) with 25 μg/ml purified PA-I, causes a profound permeability defect. This effect is also seen in the intestinal tract in vivo.

In P. aeruginosa, PA-I expression is under the regulatory control of two important systems of virulence gene regulation: the quorum sensing signaling system and the alternative sigma factor system, termed RpoS. P. aeruginosa has an exceptional ability to sense and respond to changes in its local microenvironment and switch on and off its virulence genes accordingly. Because of their importance, these two mechanisms of gene regulation are described in the following sections.

Quorum sensing signaling system. The plasticity of P. aeruginosa gene expression is achieved, in part, via the well described quorum sensing signaling system. The term quorum sensing arose from the observation that certain pathogens activate their virulence genes only when they reach a critical population density (i.e., quorum), presumably that amount necessary to mount an overwhelming attack on the host. Small molecules termed pheromones or “autoinducers” (homoserine lactones-HSLs), are self-generated by the bacterial cells and accumulate in the environment when bacteria achieve a high population density, as occurs in late stationary phase of growth. Once an intracellular threshold level of an autoinducer is reached, the autoinducer (C4-HSL, C12-HSL) binds to its cognate transcriptional regulator protein (LasR, RhlR) to activate or repress target genes. This process was first described in Vibrio fisheri as regulating the lux genes responsible for bioluminescence. P. aeruginosa possesses one of the most highly characterized models of QS, and two complete lux-like QS systems, lasR-lasI and rhlR-rhlI have been identified. Quorum sensing (QS) in P. aeruginosa controls the cell-to-cell communication pathways that direct its complex assemblage behavior including motility, biofilm synthesis, clumping, PA-I expression, and the secretion of a variety of cytotoxic exoproducts (exotoxin A, proteases, rhamnolipids, pyocyanin, elastase). Data from many models of both acute infection and chronic infection have supported the hypothesis that QS is important in P. aeruginosa pathogenesis.

RpoS signaling system. A second major regulatory system used by P. aeruginosa is the RpoS (σ^{s}) system. Bacterial σ (sigma) factors are positive regulators of gene expression that direct the initiation of transcription through direct binding to promoter sequences and recruitment of core RNA polymerase. The σ factor RpoS (σ^{s}) was originally identified in E. coli as an alternative σ factor that activates gene expression in stationary phase when cells are experiencing nutrient starvation. Now, σ^{s} is considered to be a master stress response regulator important for adaptation to a variety of conditions, including hyperthermia and oxidative stress. There is considerable evidence linking the RpoS response to QS in P. aeruginosa. The rhl quorum-sensing system has been reported to activate the transcription of rpos, and recently RpoS has been shown to affect the expression of more than 40% of all quorum-controlled genes. A master regulator like RpoS (σ^{s}) can commit a bacterial cell to a certain complex developmental program with specific temporal and spatial control being exerted by various secondary regulatory systems (FIGS. 1 and 2).

Micro array technology has enabled studies aimed at improved understanding of the molecular mechanisms of virulence gene expression in P. aeruginosa. Results from three independent studies demonstrate that both RpoS and QS play a key and interconnected role in the activation of virulence gene expression in P. aeruginosa. In addition, a key virulence gene in P. aeruginosa gut-derived sepsis, lecA (encoding PA-I) was one of only two genes shown to be directly regulated by both systems and ranked among the top 1% of QS-dependent virulence genes based on inducible transcript accumulation (Schuster et al., Mol Microbiol, 51:973-85, 2004; Winzer et al., J Bacteriol, 182:6401-11, 2000). The following is a brief description of these studies.

Greenberg and his colleagues recently performed a comprehensive analysis to identify genes regulated by RpoS in P. aeruginosa (Schuster et al.). Specifically, they compared the transcript profiles of an rpoS mutant with its parent at specific cell densities. 772 genes and 13 intergenic regions were identified that showed differential expression in stationary phase. Of the 772 genes examined, RpoS induced 504 and repressed 268. Remarkably, the PA-I gene, termed lecA, showed a 20-fold increase in expression over the rpos mutant in stationary phase of growth. In a separate microarray analysis of P. aeruginosa QS regulons, this same laboratory examined transcript abundance to exogenous HSLs (C4-HSL+C12-HSL) in wild type versus a lasRI and rhlR mutant strain. Mutants lacking lasRI and rhIR will respond poorly to their cognate QS signaling molecules, whereas wild type strains will increase transcript levels in response to exogenous HSLs. The ratio of wild type to mutant mRNA will therefore reflect increases in gene expression which are QS dependent. Among the 638 genes that showed a maximal response to exogenous HSLs of at least 2.5 fold, PA-I expression increased to a maximum of 200 fold, ranking in the top 10 inducible genes based on transcript accumulation. This study also demonstrated that lecA (which codes for the PA-I) was one of only two genes with upstream regulatory regions containing both las-rhl box sequences and RpoS sequences.

Iglewski similarly examined the effects of growth phase and environment on the P. aeruginosa QS regulon using microarray analysis in strains exposed to exogenous HSLs (Wagner et al., J Bacteriol, 185:2080-95, 2003). Media composition and oxygen availability were also examined for their effects on virulence gene expression. In this study, 616 genes were identified as being QS regulated. Only 5 genes exhibited increases of 60 fold or more in response to exogenous HSL; the PA-I was included in this high expressing group. Interestingly, the lecA gene (encoding for PA-I) was not expressed in a hypoxic/anaerobic environment.

The interconnectedness of QS and Rpos in P. aeruginosa provides a level of redundancy, additiveness, and internal feedback regulation that has armed this pathogen with signal-integrative power like no other microbe, thus accounting for its remarkable success as a lethal pathogen.

Diggle and Winzer have confirmed the above findings and identified yet a third system, the Pseudomonas Quinolone Signal (PQS) that can also activate PA-I expression by interacting directly with both the RpoS and QS systems (Diggle et al., Mol Microbiol, 50:29-43, 2003).

Microbiologists have long recognized that many bacteria activate their virulence genes in response to ambient environmental cues. In general such physico-chemical cues signal environmental stress or adversity, such as changes in redox status, pH, osmolality, and the like.

Although there has been very little work on specific membrane sensors that activate virulence gene expression in P. aeruginosa, two sensor proteins located within the cell membrane of P. aeruginosa, termed CyaB, GacS have been shown to respond to three known external signals, host cell contact, low calcium, and beet seed extract. CyaB (via cAMP) and GacS (via phosphorylation), activate the transcriptional regulators Vfr and GacA respectively, which along with the cell density sensitive PcrA, exert global regulatory influences on two central systems for virulence gene regulation in P. aeruginosa, the QS and Rpos signaling systems. Mutant strains defective in CyaB and GacS have attenuated lethality in mice following lung instillation.

Host cellular elements such as seed extract and cell contact, activate the membrane biosensors CyaB and GacS. These two component transmembrane alarm systems then activate two main global regulators of virulence, Vfr and GacA. Vfr is involved in the activation of LasRI which in turn promotes the activation of the RhlRI system of QS. GacA induces the transcription of lasR and rhlR genes, and is also implicated in the expression of rpoS. Finally a third system PQS, induces expression of both RhlR and RpoS. Thus activation of any of the membrane biosensors could lead to the expression of PA-I with the involvement of a number of different pathways.

While in vitro assays have been enormously useful and continue to provide important information on the mechanisms of bacterial pathogenesis, they cannot accurately reproduce all aspects of the host pathogen interaction, as a pathogen will encounter several radically different environments in the host at various points during infection. Consequently, a gene that seems important in in vitro studies may not be important in vivo, and genes that appear unimportant in an in vitro assay may play a critical role during a natural infection. Furthermore, it has recently been shown that bacteria growing on the surface of solid agar have a markedly different physiology from those in broth, as judged by differential regulation of nearly one-third of their functional genome. Therefore, experiments must now be designed that control for the variables of the growth environment and host environment, while at the same time allowing for measurements of gene expression patterns and phenotype analysis which are not possible in more traditional models, such as stressed mice.

Diseases and/or disorders of the lung, such as ARDS and ALI, continue to be significant problems in human and animal healthcare. Infectious and non-infectious insults to the lung continue to result in unacceptable damage to the lung. Newer and more powerful antibiotics to treat infectious insults have resulted in the emergence of highly resistant stains of bacteria for which there is no foreseeable therapy other than de-escalating their use. P. aeruginosa is now identified as one of the key microbes emerging as a resistant pathogen that poses a real and present danger to the public.

### SUMMARY

The materials and methods of the invention solve the aforementioned problem by providing high molecular weight PEG molecules to prevent, ameliorate and treat diseases and disorders arising from cell-cell barrier dysfunction, such as the endothelial and/or epithelial cell barrier dysfunction(s) characteristic of lung diseases and disorders. Acute Respiratory Distress Syndrome, or ARDS, is characterized by sudden, life-threatening lung failure with diffuse alveolar infiltrate, reduced arterial oxygenation, and pulmonary edema. Acute lung injury, ALI, is the end result of common pathways initiated by a variety of local or systemic insults. These conditions exemplify diseases and/or disorders of the lung that collectively impose a significant burden on worldwide healthcare for humans and animals. The HMW PEG compounds according to the invention are inert and non-toxic polymers that act as a surrogate mucin lining providing protection against bacterial infections of lung epithelial and endothelial cells. HMW PEG provides protection to, e.g., lung endothelium by attenuating endothelial cell (EC) activation that results in barrier dysfunction. The work described herein discloses the effects of a high molecular weight PEG (HMW PEG 15-20 kDa) on cultured human pulmonary microvessel EC exposed to barrier disruptive agents. The data show that HMW PEG induced a rapid, dose-dependent increase in transepithelial (or transendothelial) electrical resistance (TEER) similar to barrier-enhancing lipids such as sphingosine 1-phosphate (S1P). Optimal concentration of 7.5-9% HMW PEG induced a robust increase in TEER that was sustained for at least 40 hours. In comparison to S1P, which induced a 40% increase in resistance in 10-15 minutes, the maximum barrier enhancing effect of HMW PEG was achieved in about 45 minutes, but with 100% increase in resistance. With a 2-fold increase in resistance, HMW PEG is the most-potent barrier-enhancing agent tested among all the barrier-enhancing agents, such as S1P, FTY720, phospho-FTY720, and HGF. Immunofluorescence data revealed that HMW PEG altered the EC actin cytoskeleton to form a defined cortical actin ring that may help strengthen cell-cell junctional adhesion. HMW PEG rapidly induced dephosphorylation of ERK and MLC as early as 1 minute after exposure and completely inhibited thrombin-induced ERK and MLC phosphorylation. More importantly, pretreatment with HMW PEG for 1 hour attenuated thrombin-induced endothelial barrier dysfunction. In summary, HMW PEG activates a rapid, actin-associated, barrier-enhancing signal transduction pathway in EC, providing therapeutic materials and methods to prevent and reverse pulmonary diseases and disorders, such as pulmonary edema, ARDS and ALI.

The beneficial effects of HMW PEG on cell-cell barrier dysfunction have been obtained with epithelial cell barrier dysfunction, such as found in diseases of the intestine and lung, and with endothelial barrier dysfunction, such as found in the lung diseases and disorders identified herein. A greater understanding of the benefits to be obtained with the materials and methods of the invention will become apparent upon consideration of the following disclosure, stated in terms of the influence of HMW PEG on intestinal epithelial cells for ease of exposition but applicable to cell-cell interactions of epithelial cells of other tissues as well as to interactions of endothelial cells. Using a combination of in vivo and molecular methods, surgical stress has been shown to cause the release of host cell-derived Bacterial Signaling Compounds (host stress-derived BSCs) into the intestinal lumen that directly activate the virulence machinery of P. aeruginosa. The release of such host-derived BSCs, which include morphine, κ and δ opioid receptor agonists, and Interferon gamma (IFN-γ), can shift the phenotype of P. aeruginosa from that of indolent colonizer to lethal pathogen. Exposure of P. aeruginosa to host stress-derived BSCs induces the expression of the PA-I lectin/adhesin (PA-I), a key virulence gene involved in lethal gut-derived sepsis in mice. In at least some instances, induction of PA-I expression is mediated by a transcriptional regulator of virulence gene expression, MvfR. PA-I induces an epithelial and/or endothelial permeability defect to at least two potent cytotoxins of this organism, exotoxin A and elastase, causing lethal gut-derived sepsis and other disorders characterized by an epithelial cell banner dysfunction as well as disorders, e.g., acute respiratory distress syndrome, associated with an endothelial cell hairier dysfunction. The data provide evidence for a model in which opportunistic pathogens sense host stress and vulnerability by perceiving key mediators released by the host into the intestinal tract during stress, such as the stress resulting from surgery. These host stress-derived compounds directly activate critical genes in P. aeruginosa leading to enhanced virulence.

One aspect of the invention is drawn to a method of treating an epithelial and/or endothelial cell disorder of the lung comprising administering to an organism in need thereof a therapeutically effective amount of a high molecular weight polyethylene glycol-like compound having an average molecular weight of at least 8,000 daltons. In some embodiments, the polyethylene glycol-like compound is selected from the group consisting of straight-chain polyethylene glycol, branched-chain polyethylene glycol and polyethylene glycol comprising an aromatic functional group, with the latter compound being either of the straight-chain or branched chain form. Exemplary aromatic functional groups are unsubstituted and substituted phenol groups. In some embodiments, the polyethylene glycol has an average molecular weight of at least 15,000 daltons. This aspect of the invention contemplates the treatment of any epithelial and/or endothelial cell disorder of the lung; disorders amenable to the methods of the invention include acute respiratory distress syndrome, acute lung injury and lung edema. In some embodiments, the therapeutically effective amount is an amount sufficient to detectably inhibit ERK phosphorylation and/or MLC phosphorylation in an epithelial and/or endothelial cell of the organism being treated. Suitable organisms for treatment are humans, commercially useful domesticated animals such as farm animals, domesticated pets and zoo animals.

A related aspect of the invention is directed to a method of ameliorating a symptom associated with a lung cell disorder, e.g., an epithelial and/or endothelial cell disorder of the lung, comprising administering to an organism in need thereof a therapeutically effective amount of a high molecular weight polyethylene glycol-like compound having an average molecular weight selected from the group consisting of at least 8,000 daltons and at least 15,000 daltons. Another related aspect is drawn to a method of preventing an epithelial and/or endothelial cell disorder of the lung comprising administering to an organism at risk a prophylactically effective amount of a high molecular weight polyethylene glycol-like compound having an average molecular weight selected from the group consisting of at least 8,000 daltons and at least 15,000 daltons. For either of these aspects, suitable lung disorders include acute respiratory distress syndrome, acute lung injury and lung edema. A suitable organism for each of these aspects of the invention is described in the preceding paragraph and includes humans. For the method of preventing an epithelial and/or endothelial cell disorder of the lung, it is contemplated that a prophylactically effective amount is an amount sufficient to detectably inhibit ERK phosphorylation or MLC phosphorylation in an epithelial and/or endothelial cell of the organism at risk.

Another related aspect of the invention is a kit comprising a high molecular weight polyethylene glycol (HMW PEG) as described herein and a set of instructions for administering the HMW PEG to treat, prevent or ameliorate a lung disorder. In some embodiments, the kit comprises HMW PEG that has a molecular weight selected from the group consisting of at least 8,000 daltons and of at least 15,000 daltons.

In another aspect, the invention provides a method of preventing, inhibiting, reversing, or diminishing a paracellular barrier disruption comprising administering to an organism in need thereof a therapeutically effective amount of a high molecular weight polyethylene glycol-like compound having an average molecular weight of at least 8,000 daltons. The methods according to this aspect of the invention include methods wherein the polyethylene glycol-like compound has an average molecular weight of at least 15,000 daltons. In some embodiments, the paracellular barrier disruption is a paracellular barrier disruption of endothelial cells or epithelial cells, for example lung endothelial cells and/or lung epithelial cells. Embodiments of this invention include methods wherein the paracellular barrier disruption is associated with a lung disorder, such as acute respiratory distress syndrome, acute lung injury, and/or pulmonary edema. In some embodiments, the paracellular barrier disruption is produced by an edemagenic agent selected from the group consisting of thrombin and lipopolysaccharide. Also contemplated are methods further comprising enhancement of a cortical actin cytoskeleton. In some embodiments, the high molecular weight polyethylene glycol inhibits phosphorylation of a protein selected from the group consisting of ERK and MLC in the cell.

In another aspect, the invention provides a method of treating a lung epithelial and or endothelial cell disorder characterized by a cell barrier dysfunction (partial or complete barrier dysfunction) comprising administering to an organism in need thereof a therapeutically effective amount of a compound selected from the group consisting of an opioid receptor antagonist, an interferon-γ antagonist, an MvfR antagonist, a regulator of MvfR expression, a PA-I antagonist, a negative regulator of PA-I expression, an endomorphine-1 antagonist, an endomorphine-2 antagonist, an antagonist to δ opioid agonist BW373U86 and the modulator identified by one of the above-described screening methods. In preferred embodiments, the organism in need is a human patient. Contemplated as suitable for treatment is a disorder selected from the group consisting of gut-derived sepsis, a burn injury, neonatal necrotizing enterocolitis, severe neutropenia, toxic colitis, inflammatory bowel disease, enteropathy, transplant rejection, pouchitis, pig belly, Pseudomonas-medvaled opthalmologic infection, Pseudomonas-mediatQd otologic infection and Pseudomonas-mediated cutaneous infection. In some embodiments, the treatment method further comprises administration of a high molecular weight polyethylene glycol-like compound, such as a polyethylene glycol having an average molecular weight of at least 15 kilodaltons.

In some embodiments of the treatment methods of the invention, an opioid receptor antagonist is administered that is an antagonist of a κ-opioid receptor agonist selected from the group consisting of U-50488, U-69593, enadoline, ethylketocyclazocine, salvinorin A and asimadoline. In other embodiments, an opioid receptor antagonist is selected from the group consisting of nor-binaltorphimine, 5′-guanidinonaltrindole, nalmefine, naltrindole, an indolmorphinan, naltrexone and MR2266 ([(−)-(1R,5R, 9R)-5,9-diethyl-2-(3-furylmethyl)-2′-hydroxy-6,7-benzomorphan).

In yet other embodiments, a modulator suitable for use in the treatment methods include interferon-γ antagonists, opioid receptor (including κ, δ, and μ opioid receptor) antagonists, an endomorphine-1 (9E1; Tyr-Pro-Trp-Phe-NH₂) antagonist, and an endomorphine-2 (E2; Tyr-Tyr-Pro-Phe-Phe-NH₂) antagonist. An exemplary δ-opioid receptor antagonist is an antagonist to the δ-opioid receptor agonist, BW373U86. Preferred modulators are interferon-γ antagonists and κ-opioid antagonists. An exemplary interferon-γ antagonist is an antibody specifically recognizing interferon-γ, including polyclonal, monoclonal, chimeric, humanized, single-chain and all other forms of antibody known in the art. Contemplated examples of κ-opioid antagonists are nor-binaltorphimine (nor-binaltorphamine, Magers et al., J. Pharmacol. Exp. Ther. 305:323-330 (2003)), 51-guanidinonaltrindole (Magers et al.), 5′-acetamidinoethylnatrindole (Magers et al.), nalmefine (Culpepper-Morgan et al., Life Sci. 56:187-192 (1995)), naltrindole and indolmorphinans, i.e., compounds comprising a derivatized indole moiety of naltrindole (Stevens et al., J. Med. Chem. 43:2759-2769 (2000)), naltrexone (Craft et al., J. Pharmacol. Exp. Ther. 260:327-333 (1992)), and MR2266 ([(−)-(1R,5R, 9R)-5,9-diethyl-2-(3-furylmethyl)-2′-hydroxy-6,7-benzomorphan (Fanselow et al., Beh. Neurosci. 103:663-672 (1989). Additionally contemplated as useful κ-opioid antagonists are specific anti-κ-opioid agonist-recognizing antibodies, including polyclonal, monoclonal, chimeric, humanized, single-chain and all other forms of antibody known in the art. For example, an antibody specifically recognizing the U-50,488 κ-opioid agonist is contemplated by the invention, as are antibodies of any form that specifically recognize any known κ-opioid agonist, including but not limited to U-69,593 (Filizola et al., J. Comput. Aided Molec. Des. 15:297-307 (2001)), U-62,066 (spiradolinc, Pitts et al., J. Pharmacol.Exp. Ther. 271:1501-1508 (1994)), enadoline (C1-977, Pitts et al.), ethylketocyclazocine (Mello et al., J. Pharmacol. Exp. Ther. 286:812-824 (1998)), salvinorin A, and asimadoline (EMD 61753, Machalska et al., J. Pharmacol. Exp. Ther. 290:354-361 (1999)). Other κ-opioid antagonists according to the invention are antibodies of any form that specifically recognize a P. aeruginosa κ-opioid receptor. Each of the references cited in disclosing antagonist modulators is expressly incorporated herein by reference for disclosures relating to such antagonists.

Further contemplated in the treatment methods of the invention are modulators of MvfR-mediated virulence gene expression. Exemplary embodiments of these modulators include compounds that bind to at least one of the substrate binding domain or the DNA binding domain of MvfR. Related embodiments include treatment methods comprising modulators of MvfR-mediated virulence gene expression that inhibit expression of MvfR. The invention also comprehends treatment methods comprising the administration of a therapeutically effective amount of a modulator of PA-I lectin/adhesin activity. Exemplary modulators of PA-I activity are inhibitors of PA-I activity, such as specific anti-PA-I antibodies of any form. Further, such modulators include compounds that reduce the level of expression of PA-I.

Still other treatment methods according to the invention involve administration of a therapeutically effective amount of an anti-sense oligonucleotide capable of specifically hybridizing to a nucleic acid comprising a sequence required for the expression of an opioid receptor, OprF, or MvfR. Related treatment methods involve administration of a therapeutically effective amount of a compound capable of inducing post-transcriptional gene silencing, such as administration of a vector for intracellular expression of a double-stranded RNA homologous to, e.g., the PA-I coding region or an MvfR coding region, or administration of the dsRNA itself, in an RNAi approach to treatment.

In yet another aspect, the invention provides a method of reducing the risk of developing a disorder characterized by a lung epithelial and/or endothelial cell barrier dysfunction comprising administering to an organism at risk of developing the disorder a prophylactically effective amount of a compound selected from the group consisting of an opioid receptor antagonist, an interferon-γ antagonist, an MvfR antagonist, a regulator of MvfR expression, a PA-I antagonist, a negative regulator of PA-I expression, an endomorphine-1 antagonist, an endomorphine-2 antagonist, an antagonist to δ opioid agonist BW373U86 and the modulator described herein. In some embodiments, an opioid receptor antagonist is selected from the group consisting of a U-50488 antagonist, a U-69593 antagonist, an enadoline antagonist, an ethylketocyclazocine antagonist, a salvinorin A antagonist, an asimadoline antagonist, nor-binaltorphimine, 5′-guanidinonaltrindole, nalmefine, naltrindole, an indolmorphinan, naltrexone and MR2266 ([(−)-(1R,5R, 9R)-5,9-diethyl-2-(3-furylmethyl)-2′-hydroxy-6,7-benzomorphan. In preferred embodiments, the organism at risk is a human being, such as a human patient. The methods are contemplated as suitable for reducing the risk of acquiring or developing a disorder of the lung, such as acute respiratory distress syndrome. Related aspects of the invention provide methods for reducing the risk of such disorders comprising administration of a therapeutically effective amount of a high molecular weight polyethylene glycol-like compound, such as a polyethylene glycol having an average molecular weight of at least 15 kilodaltons, alone or in combination with the administration of a therapeutically effective amount of a compound described above.

Another aspect of the invention is drawn to a method of reducing a symptom associated with a lung epithelial and/or endothelial cell barrier disorder, comprising administering to a patient in need thereof a compound selected from the group consisting of an opioid receptor antagonist, an interferon-γ antagonist, an MvfR antagonist, a regulator of MvfR expression, a PA-I antagonist, a regulator of PA-I expression, an endomorphine-1 antagonist, an endomorphine-2 antagonist, an antagonist to δ opioid agonist BW373U86 and a modulator identified according to one of the above-described screening methods, wherein the compound is administered in an amount effective to reduce at least one symptom of the disorder. In some embodiments, an opioid receptor antagonist is an antagonist of a κ-opioid receptor agonist selected from the group consisting of U-50488, U-69593, enadoline, ethylketocyclazocine, salvinorin A and asimadoline. In some embodiments, an opioid receptor antagonist is selected from the group consisting of nor-binaltorphimine, 5′-guanidinonaltrindole, nalmefine, naltrindole, an indolmorphinan, naltrexone and MR2266 ([(−)-(1R,5R, 9R)-5,9-diethyl-2-(3-furylmethyl)-2′-hydroxy-6,7-benzomorphan). Related aspects of the invention provide methods for reducing a symptom of such disorders comprising administration of a therapeutically effective amount of a high molecular weight polyethylene glycol-like compound, such as a polyethylene glycol having an average molecular weight of at least 15 kilodaltons, alone or in combination with the administration of a therapeutically effective amount of a compound described above.

In all of the aspects of the invention drawn to methods of treating a disorder, methods of modulating the activity of PA-I lectin/adhesin, methods of modulating the activity of MvfR, methods of reducing the risk of developing an epithelial and/or endothelial cell barrier disorder, or methods of reducing a symptom associated with an epithelial and/or endothelial cell barrier disorder disclosed herein, administering the therapeutically or prophylactically active compound, as disclosed herein (e.g., a modulator identified by a screening method according to the invention), is achieved by any mode that is known in the art, and in particular, parenteral delivery, oral delivery, subcutaneous delivery, transcutaneous delivery, intramuscular delivery, intravenous delivery, topical delivery and nasal inhalation. The invention comprehends administering the active compound by direct routes, such as by nasal inhalation. In addition, the invention comprehends treatment of any disorder caused, at least in part, by a microbial pathogen such as P. aeruginosa by administering an active compound through conventional systemic routes, including intravitreously, intracerebroventricularly, and topically (e.g., opthalmologically, otologically, cutaneously), at levels sufficient to achieve therapeutically useful systemic levels of the active compound. More generally, the invention contemplates any route known in the art to be suitable for preventing or treating such disorder (e.g., infections), including direct delivery as well as delivery by a systemic mode of delivering the active compound. A number of administration routes are suitable for either localized administration or systemic administration, for which dosages would be optimized using techniques routine in the art

Yet another aspect according to the invention is a method of treating neonatal necrotizing enterocolitis comprising administering to an organism in need thereof a therapeutically effective amount of a high molecular weight polyethylene glycol-like compound comprising an average molecular weight of at least 8,000 daltons. In some embodiments, the polyethylene glycol-like compound is selected from the group consisting of straight-chain polyethylene glycol, branched-chain polyethylene glycol and polyethylene glycol comprising an aromatic functional group, with the latter compound being either of the straight-chain or branched chain form, as noted above. Exemplary aromatic functional groups are unsubstituted and substituted phenol groups. In some embodiments, the polyethylene glycol has an average molecular weight of at least 15,000 daltons. In some embodiments, the therapeutically effective amount is an amount sufficient to detectably inhibit ERK phosphorylation or MLC phosphorylation in an epithelial and/or endothelial cell of the organism being treated, such as an intestinal epithelial cell or a lung endothelial cell. Suitable organisms for treatment are humans, commercially useful domesticated animals such as farm animals, domesticated pets and zoo animals.

In a related aspect, the invention provides a method of ameliorating a symptom associated with neonatal necrotizing enterocolitis comprising administering to an organism in need thereof a therapeutically effective amount of a high molecular weight polyethylene glycol-like compound having an average molecular weight of at least 8,000 daltons. Yet another related aspect is directed to a method of preventing neonatal necrotizing enterocolitis comprising administering to an organism at risk a prophylactically effective amount of a high molecular weight polyethylene glycol-like compound having an average molecular weight of at least 8,000 daltons. For either of these aspects, the methods comprehend high molecular weight polyethylene glycol-like compounds that are polyethylene glycols having average molecular weights of at least 15,000 daltons. A suitable organism for each of these aspects of the invention is described above and includes humans. A therapeutically or prophylactically effective amount is contemplated as including an amount sufficient to detectably inhibit ERK phosphorylation or MLC phosphorylation in an epithelial cell, e.g., an intestinal epithelial cell, or an endothelial cell, e.g., an endothelial lung cell, of the organism in need or at risk.

Other features and advantages of the present invention will be better understood by reference to the following detailed description, including the drawing and the examples.

### BRIEF DESCRIPTION OF THE DRAWING

FIG. 1. PA-I expression is regulated by QS and RpoS. The regulatory region upstream of the lecA gene contains both lux box and rpos consensus sequences. The RhlRI system of QS is a key factor in the regulation of PA-I. Diffusible C4-HSL and C12-HSL are self generated by bacteria in response to population density. C4-HSL binds to the dimerized regulator protein RhlR, and then the RhlR-C4-HSL-RhlR complex binds to DNA at the site of lux box upstream of pa-I gene, thereby activating the promoter for RNA polymerase. The promoter of pa-I is specifically recognized by the alternative σ^{s} factor (Rpos). The binding of the initiation factor σ^{s} to RNA polymerase core enzyme results in the formation of the active form of RNA polymerase that binds to the pa-I promoter initiating the process of transcription. PA-I expression is both QS and RpoS dependent as mutant strains lacking RhlR, RhlI, or RpoS do not produce PA-I.

FIG. 2. Membrane activation of PA-I expression by host cell components. Host cellular elements such as seed extract and cell contact, activate the membrane biosensors CyaB and GacS. These two component transmembrane alarm systems then activate two main global regulators of virulence, Vfr and GacA. Vfr is involved in the activation of LasRI which in turn promotes the activation of the RhlRI system of QS. GacA induces the transcription of lasR and rhlR genes, and is also implicated in the expression of rpoS. Finally a third system PQS, induces expression of both RhlR and RpoS. Thus activation of any of the membrane biosensors could lead to the expression of PA-I with the involvement of a number of different pathways.

FIG. 3. PA-I GFP reporter strain. Plasmid contains QS lux box and RpoS consensus sequences upstream of the PA-I gene.

FIG. 4. Exposure of P. aeruginosa to IFNγ induces PA-I expression and the expression of rhlI. For controls (lane 1, 2) bacteria are suspended in media I (Tryptic soy broth) and media II (DMEM+10% FBS ). Lane 3, 4 are mRNA levels from bacteria exposed to IFNγ and TNFα in DMEM.

FIG. 5. IFN-γ binds to P. aeruginosa in a dose-dependent manner. A. Binding characteristics of recombinant human (rh) IFN-γ to whole bacterial cells (PA27853) by ELISA. B. Alexa 594 labeled antibody staining of IFN-gamma treated PA27853. C. FACS analysis to quantitate % binding of IFN-γ to individual cells of PA27853. (*P<0.001).

FIG. 6. IFN γ binds to solubilized membrane proteins of P. aeruginosa and E. coli A. Binding characteristics of IFN-gamma to solubilized PA27853 membrane fractions by ELISA. B. Solubilized membrane proteins were run on non-denaturing gels and demonstrated a single immunoreactive band at 30 kDa. C. Immuno-precipitation of bacterial membrane proteins showing single immunoreactive band at 25 kD. BSA, Bovine serum albumin.

FIG. 7. IFN-γ enhances barrier dysregulating effect of PA27853 against Caeo-2 cells. PA27853 was apically inoculated onto Caco-2 cells in the presence and absence of IFN-γ. Neither media nor IFN-γ alone altered TEER, whereas PA27853 decreased TEER by 60%; an effect which was enhanced in the presence of IFN-γ. *P<0.01 versus PA27853 at 5 hour time point.

FIG. 8. Morphine induces a 4-fold increase in PA-I mRNA in PA27853. Quantitative RT-PCR of PA27853 exposed to morphine (13 μM) and its cognate quorum sensing signaling molecule, C4-HSL (100 μM). *P<0.001.

FIG. 9. κ and δ opioid receptor agonists induce a 4-fold increase in fluorescence in PA-I reporter strains and enhance the ability of PA27853 to alter epithelial barrier function. A. PA-I GFP reporter strain PA27853/PLL-EGFP was incubated in FIDMEM media containing 60 μM of two pure p opiate agonists (E-1, E-2), a pure κ agonist, and a pure δ agonist. Fluorescence was measured over 9 hours. B. Opiate agonists induced PA27853 to display enhanced barrier dysregulating properties against MDCK cells. *P<0.001. Opioid receptor agonists alone had no effect on monolayer TEER.

FIG. 10. κ and δ opioid receptor agonists increase the production of biofilm in PA27853. Biofilm, a quorum sensing regulated virulence determinant, is markedly increased in PA27853 in the presence of κ and δ opioid receptor agonists. This effect was dose dependent and especially increased with the δ agonist. (*P<0.01) Right panel shows actual biofilm assay in presence of varying doses of δ agonist BW373U86.

FIG. 11. PA-I protein is abundantly expressed in PAO1 when strains are exposed to opioid receptor agonists. PA-I protein increased significantly in PAO1 in response to opioids. In these experiments the δ agonist, BW373U86, induced PA-I protein to the same degree as C4-HSL.

FIG. 12. IFN-γ induces the expression of the PA-I lectin in P. aeruginosa. (A) The GFP-PA-I reporter strain, PLL-EGFP/27853, was exposed to media from activated T cells. PA-I expression was assessed in PA-I reporter strains by measuring fluorescence over time and expressed as relative fluorescent units (RFU/control). The PA-I inducing effect of the media from activated T cells (maximum at 7 hours) was eliminated by immuno-depletion of the media with anti-IFN-γ antibody. (B) Optical density of P. aeruginosa grown in 96 wells at slow speed showed P. aeruginosa reached stationary growth phase at 6 hours. (C) Only IFN-γ induced PA-I promoter activity after exposure of various cytokines to the GFP-PA-I reporter strain. (D) P. aeruginosa (PAO1) was incubated with 200 ng/ml IFN-γ, TNF-α, IL-2, IL-4, IL-8, IL-10 in cell culture media for 4 hours, RNA was extracted, and PA-I mRNA measured by Northern blot. Induction of PA-I mRNA was observed in the presence of IFN-γ and C₄-HSL, but not TNF-α, IL-2, IL-4, IL-8, or IL-10. (E) P. aeruginosa was harvested at 2 hours (OD₆₀₀=1.0) and 4 hours (OD₆₀₀=1.8) after exposure to 200 ng/ml IFN-γ in cell culture media. Northern blot showed PA-I mRNA was significantly increased by IFN-γ exposure at early stationary phase of growth (OD₆₀₀=1.8). (F) Growth phase dependent expression of PA-I lectin measured by immunoblot. PA-I expression was induced following exposure to IFN-γ during stationary phase of growth-an effect not observed during log phase growth. (G) Dose dependent enhancement of PA-I expression following exposure to IFN-γ for 6 hours.

FIG. 13. The presence of rhlI and rhlR, core quorum sensing signaling elements in P. aeruginosa, are required for the PA-I expression and pyocyanin production in response to IFN-γ. (A) P. aeruginosa was harvested at 2 hours (OD₆₀₀=1.0) and 4 hours (OD₆₀₀=1.8) following incubation 200 ng/ml IFN-γ in cell culture media. Northern blot demonstrated that IFN-γ increased rhlI mRNA levels significantly. (B) IFN-γ but not TNF-α induced the transcription of rhlI mRNA. (C) The gene product of rhlI, C₄-HSL, a key diffusible quorum sensing signaling molecule, was measured by the luminescence reporter strain pSB536 in PAO1 supernatant and was increased following exposure to IFN-γ. (D) Pyocyanin, an additional quorum sensing dependent virulence factor, was also up-regulated in PAO1 in the presence of 100 ng/ml of IFN-γ. (E) (F) Pyocyanin production required the presence of rhlI and rhlR as mutants did not produce pyocyanin when exposed to IFN-γ. Exposure of the rhlR mutant strain to C₄-HSL did not restore PCN production.

FIG. 14. Identification of the IFN-γ binding site to solubilized membrane fractions of P. aeruginosa (PAO1). (A) Whole cells of P. aeruginosa were coated onto microtiter wells. IFN-γ was added to the wells and binding detected by standard ELISA assay. Dose dependent binding to P. aeruginosa was observed. (B) Epimicrography of immunofluorescence of IFN-γ bound to whole cells of P. aeruginosa. Binding was detected using biotin labeled anti-IFN-γ antibody and fluorescence Alexa 594 labeled streptavidin. Bar: 5 μm. (C) Coating of solubilized membrane protein onto microtiter wells demonstrated that IFN-γ binds to solubilized membrane proteins from P. aeruginosa. (D) Solubilized membrane proteins separated by non-denature PAGE gels were detected using IFN-γ as the first antibody. Representative Immunoblot (n=3) demonstrated dose-dependent IFN-γ binding to a single 35 kDa solubilized membrane protein. (E) Immunoprecipitation of the solubilized membrane protein with IFN-γ and anti-IFN-γ antibody resulted in a distinctive band. (F) MS/MS spectra of the tryptic peptide (T16) digested protein immunoprecipitated from the solubilized membrane proteins by ESI-TRAP-Electrospray LC-MSMS Ion Trap identified as OprF (outer membrane protein F).

FIG. 15. IFN-γ binds to OprF and induces PA-I expression. (A) ELISA binding assays of IFN-γ to solubilized membrane protein from wild-type P. aeruginosa (PAO1) and the OprF knockout mutant strain 31899 of P. aeruginosa showing attenuated IFN-γ binding to the solubilized membrane protein from the mutant strain. (B) Immunoprecipitation of solubilized membrane proteins with IFN-γ from OprF mutant strain 31899 demonstrating absence of the 35 kDa band seen with the parent wild-type strain (PAO1). (C) PA-1 protein expression measured by immunoblot in wild-type (PAO1) and mutant strains (31899, 43114) of P. aeruginosa exposed to 200 ng/ml IFN-γ demonstrating an inability of IFN-γ to enhance the expression of PA-I in the OprF mutant strains. (D) Wild-type strain (PAO1) and OprF mutant strains (31899, 43114) carrying the GFP-PA-I fusion plasmid were incubated with 200 ng/ml IFN-γ and fluorescence assessed over time. Results demonstrate a lack of enhanced PA-I expression in mutants exposed to IFN-γ (E) Reconstitution of OprF in mutant strain 31899 demonstrating re-establishment of the responsiveness of PA-I expression to IFN-γ. (F) Anti-OprF antibody (polyclonal: pAb, monoclonal: mAb) was coated onto microtiter plate. The complexes (OprF and IFN-γ, IFN-γ and Lys (lysozyme), OprF and TNF-α) were added and detected by biotin labeled anti-IFN-γ antibody. ELISA assay demonstrated that human IFN-γ binds to purified OprF. Results are a representative experiment of 3 independent studies.

FIG. 16. Epifluorescence photomicrographs of IFN-γ bound to whole cells of P. aeruginosa. P. aeruginosa was incubated with IFN-γ and detected by biotin labeled anti-IFN-γ antibody and Alexa 594 labeled streptavidin. Controls included: P. aeruginosa; Streptavidin-Alexa 594 only; IFN-γ+Streptavidin-Alexa 594; Anti-IFN-γ antibody+streptavidin-Alexa 594. Bar: 5 μm.

FIG. 17. Binding characteristics of the IFN-γ to membrane fractions of P. aeruginosa (PAO1). (A) IFN-γ binding capacity to P. aeruginosa harvested at various growth phases were measured by ELISA binding assay. P. aeruginosa at log growth phase has attenuated binding capacity to IFN-γ. (B) Membrane and cytosolic fractions of P. aeruginosa were coated onto microtiter wells. ELISA binding assay demonstrated that IFN-γ preferentially binds to P. aeruginosa membrane fraction. (C) Membrane fractions were digested with proteinase K and retested for IFN-γ binding by ELISA. (D) The binding characteristics of TNF-α, IL-2, IL-4, IL-10, EGF and TGF-β to membrane fractions of P. aeruginosa were determined by ELISA binding assay. Only IFN-γ bound to P. aeruginosa membrane fractions.

FIG. 18. κ-opioid receptor agonists U-50,488 and dynorphin induce PCN biosynthesis in P. aeruginosa 27853 and PAO1. (A) PAO1 grown overnight in tryptic soy broth (TSB) (control) in the presence of 1 mM U-50,488, BW373U86 or morphine demonstrated a bright green appearance in response to U-50,488. (B) PAO1 and 27853 grown overnight in TSB in the presence of varying concentrations of U-50,488 demonstrating increased pyocyanin (PCN) in a concentration-dependent manner. Data are mean±SD (n=3). (C) PAO1 grown overnight in TSB in the presence of varying concentrations of dynorphin demonstrated increased PCN production in a dose-dependent manner. Data are mean±SD (n=5). (D) PAO1 grown overnight in TSB in the presence of 200 μM U-50,488 and varying concentrations of a specific κ-opioid receptor antagonist nor-binaltorphimine (NOR). Data are mean±SD (n=3).

FIG. 19. PCN production dynamics. Dynamic tracking of PCN production in strain PAO1 grown in TSB supplemented with 200 μM of U-50,488 or 200 μM C4-HSL demonstrated a shift in the onset of PCN production at lower cell densities. Data are mean±SD (n=3).

FIG. 20. U-50,488 induces PCN production via elements of QS. (A) Proposed pathway of PCN regulation in P. aeruginosa PAO1. PCN biosynthesis is regulated by the RhlRI system (M. R. Parsek and E. P. Greenberg, Proc Natl Acad Sci USA 97, 8789 (2000)) which consists of the transcriptional regulator, RhlR, and the synthase RhlI which produces C4-homoserine lactone (C4-HSL). The RhlRI system is regulated by LasRI consisting of the transcriptional activator, LasR, and the synthase LasI which produces 3-oxo-C12-homoserine lactone (3OC12-HSL) (T. R. de Kievit, Y. Kakai, J. K. Register, E. C. Pesci and B. H. Iglewski, FEMS Microbiol Lett 212, 101 (2002)). These two core QS systems (LasRI, RhlRI) can be activated by the proximal transcriptional regulator GacA (C. Reimmann, et al., Mol Microbiol 24, 309 (1997)). Another proximal transcriptional regulator MvfR activates 3OC12-HSL production (38), and regulates the synthesis of the Pseudomonas quinolone signal (PQS) via expression of the phnAB and pqsABCDE operons controlling the synthesis of the PQS precursors, anthranilate and 4-hydroxy-2-heptylquinoline (HHQ). PQS has also been shown to induce rhlI (S. L. McKnight, B. H. Iglewski and E. C. Pesci, J Bacteriol 182, 2702 (2000)). PQS synthesis is also regulated by LasRI via activation of pqsH—a step that is necessary for the conversion of HHQ to PQS (E. Deziel, et al., Proc Natl Acad Sci USA 101, 1339 (2004)). (B) Effect of U-50,488 on PCN production in mutants defective in core elements of QS. (C, D) Effect of U-50,488 (C) and C4-HSL (D) on PCN production in mutants defective in the proximal transcriptional activators GacA and MvfR. Data are mean±SD (n=3).

FIG. 21. MvfR plays a key role in the effect of U-50,488 and C4-HSL on PCN production. (A) Complementation of strain 13375 with the mvfr gene restored PCN production at a level above that of the parental strain PAO1 (ΔMvfR, strain 13375 harboring blank plasmid pUCP24; ΔMvfR/mvfR, strain 13375 harboring mvfr gene on pUCP24). (B) Complementation of strain PAO6281 with the gacA restored PCN production at a level above that of the parental strain PAO1 (ΔGacA. strain PAO6281 harboring blank plasmid pUCP24; ΔGacA/gacA, strain PAO6281 harboring gacA gene on pUCP24). (C) Overnight culture of ΔMvfR/mvfR in TSB showing increased PCN production in the presence of 1 mM of U-50,488 or 100 μM of C4-HSL. (D) ΔGacA/gacA grown overnight in TSB, showing decreased PCN production in the presence of 1 mM of U20 50,488 or 100 μM of C4-HSL. (E) Dynamic tracking of PCN production in strain ΔMvfR/mvfR grown in TSB supplemented with 200 μM of U-50,488 or C4-HSL demonstrating a shift in the onset of PCN production similar to that of the parental strain PAO1 (FIG. 19). (F) Dynamic tracking of PCN production in strain in ΔGacA/gacA grown in TSB supplemented with 200 μM of U-50,488 and C4-HSL demonstrating no shift in the onset of PCN production in the presence of C4-HSL or U-50,488. Data are mean±SD (n=3).

FIG. 22. Intact substrate-binding and DNA-binding domains of MvfR are required for U-50,488- and C4-HSL-inducing effect on PCN production. (A) Specific LysR DNA binding domain (DBD) from 6 to 64 amino acids (blue) and substrate-binding domain (SBD) from 156 to 293 amino acids (pink) are shown in Mvfr. Truncations of C and N terminus of mvfR produced several versions of truncated MvfRs. (B) C-terminal truncations of 33 (1-299) and 39 (1-293) amino acids do not affect the functionality of MvfR as judged by ability of U-50,488 and C4-HSL to induce PCN production. However, C-terminal truncation of 40 (1-292) and additional amino acids (1-291, 1-283) that affected the substrate-binding domain, completely abrogated PCN production and the response to C4-HSL or U-50,488. Similarly, N-terminal truncation of 120 amino acids (121-322) that affected the DNA-binding domain, abrogated PCN production. Data are mean±SD (n=3).

FIG. 23. PQS plays an important role in the pathway by which U-50,488 enhances PCN production in PAO1. (A) Effect of U-50,488 on PCN production in ΔPhnA, a mutant defective in the synthesis of anthranilate, ΔPsqA, a mutant defective in the synthesis of 4-hydroxy-2-heptylquinolone (HHQ), and ΔPhzA1. (B) Use of the compound methyl anthranilate (MA), a known inhibitor of PQS synthesis, attenuated the ability of U-50,488 to enhance PCN production in PAO1. Data are mean±SD (n=3).

FIG. 24. U-50,488 induces biofilm and PA-IL production in P. aeruginosa PAO1 and 27853. (A) Exposure of PAO1 to U-50,488 showed a dose dependent increase in biofilm formation. Data are mean±SD (n=5). (B) Dynamic tracking of PA-IL expression by in the GFP-PA-IL reporter strain 27853/PLL-EGFP (L. Wu, et al., Gastroenterology 126, 488 (2004)) showed a time dependent enhancement of PA-IL expression in response to U-50,488. Data are mean±SD (n=3). (C) Immunoblot analysis of PAO1 exposed to 100 μM of U-50,488 demonstrated an increase in PA-IL protein expression at cell densities of 1.8 (OD_{600 nm}).

FIG. 25. PEG 15-20 attenuates the effect of U-50,488 on PCN production in P. aeruginosa. PAO1 was incubated overnight in TSB (control) or in a 5% PEG 15-20 solution in TSB in the presence of 0.5 mM U-50,488 or 0.2 mM C4-HSL (positive control). PCN was extracted and measured at OD_{520 nm}. Data are mean±SD (n=3).

FIG. 26. Adenosine exerts a direct effect on PA-I promoter activity in GFP-PA-I reporter strain PLL/EGFP. Dilutions of adenosine in HDMEM were tested against the GFP-PA-I reporter strain. Although PA-I promoter activity was enhanced in response to 10 mM of adenosine (*P< 0.05, ***P< 0.001), it required at least 6 hours of exposure to the compound, much longer than that observed with hypoxic media alone.

FIG. 27. Dose response of HMW PEG on HLMVEC. EC were cultured on gold microelectrodes and subjected to transendothelial electrical resistance measurements to detect changes in barrier permeability. Increases in resistance corresponded to tightening of junctional adhesion resulting in enhanced barrier function, while decreases in resistance corresponded to barrier dysfunction and increased paracellular permeability. Addition of HMW PEG at time=0 minutes resulted in a rapid and sustained increase in barrier enhancement.

FIG. 28. HMW PEG induced cortical actin and VE-cadherin formation. HLMVEC were stimulated with 0, 7.5, or 10% HMW PEG dissolved in EBM. After a 1-hour treatment, cells were fixed and stained with Texas Red-phalloidin to visualize F-actin (top 3 panels) or VE-cadherin antibodies to detect VE-cadherin (bottom 3 panels).

FIG. 29. HMW PEG inhibited ERK and MLC phosphorylation. S1P, a barrier-enhancing agent, has been shown to result in activation of specific signal transduction pathways as detected by phosphorylation of ERK and MLC. Thus, the effects of HMW PEG at various concentrations and treatment times on ERK and MLC phosphorylation were examined via Western blot analyses. In contrast to S1P, HMW PEG potently inhibited basal phosphorylation of ERK (shown) and MLC.

FIG. 30. HMW PEG blocked thrombin-induced ERK phosphorylation and attenuated MLC phosphorylation. Since HMW PEG inhibited basal ERK and MLC phosphorylation, the ability of HMW PEG to inhibit thrombin-induced ERK and MLC activation was examined. Cells were treated with PEG (7.5%, 30 minutes) and challenged with thrombin (1 U/ml, 5 minutes). Lysates were processed for Western blot analyses and probed using phospho-specific antibodies to ERK and MLC. HMW PEG completely blocked thrombin-induced ERK phosphorylation and potently attenuated thrombin-induced MLC phosphorylation. To determine if the HMW PEG treatment had persistent effect after removal, cells were pretreated with HMW PEG for 5 minutes followed by replacement with EBM. After 25 minutes incubation, cell lysates were harvested and processed for Western blots. The presence of HMW PEG was found necessary to induce the inhibitory effects on ERK and MLC phosphorylation.

FIG. 31. HMW PEG reconstituted in EBM enhanced barrier function but did not protect from thrombin-induced barrier dysfunction. The potential for HMW PEG to block thrombin-induced barrier dysfunction was investigated. Cells grown on gold microelectrodes were subjected to ECIS measurements to determine effects on barrier function. HMW PEG quickly and potently enhanced barrier resistance, but did not provide barrier protection upon thrombin challenge. The progressive decrease in resistance to thrombin stimulation indicated that serum-free conditions and HMW PEG had an unfavorable synergistic effect. HMW PEG reconstituted in serum-free medium induced quiescence and hypersensitivity in EC.

FIG. 32. HMW PEG reconstituted in growth media induced dose-dependent increases in endothelial barrier enhancement. In comparison to HMW PEG reconstituted in serum-free medium, HMW PEG in serum-containing medium also induced dose-dependent increases in electrical resistance measurements. The magnitudes of the increases appeared lower (approximately 1000 ohm) for equivalent HMW PEG concentrations, indicating a decrease in cell sensitivity.

FIG. 33. HMW PEG reconstituted in growth media protects cells from thrombin-induced barrier disruption. Since serum-free conditions do not favor prolonged treatment of cells with HMW PEG and an agonist, the effect of HMW PEG in serum conditions was examined. Cells were pretreated with HMW PEG (8%, 1 hour) reconstituted in growth medium, and subsequently challenged with thrombin (1 U/ml). HMW PEG induced a rapid and sustained barrier enhancement similar to that seen under serum-free conditions. Furthermore, HMW PEG in EGM was able to provide barrier protection from thrombin by preventing a decrease in resistance below 3500 ohm. In contrast to serum-free conditions, barrier restoration upon thrombin challenge returned to the elevated level of 4500-5000 ohm, indicating the importance of serum conditions for prolonged enhancement of barrier function provided by HMW PEG.

FIG. 34. PEG decreased intracellular calcium concentration and blocked S1P-induced, but not thrombin-induced, calcium spike. HPAECs grown on glass cover slips (Digilab, Randolph, Mass.) were loaded with 5 μM Fura-2 AM (Molecular Probes, Eugene, Oreg.) in 1 ml of basic medium (116 mM NaCl, 5.37 mM KCl, 26.2 mM NaHCO₃, 1.8 mM CaCl₂, 0.81 mM MgSO₄, 1.02 mM NaHPO₄,5.5 mM glucose, 10 mM HEPES/HCl, pH 7.4). Cells were incubated at 37° C. for 15 minutes in 95% O₂ and 5% CO₂, rinsed twice, and inserted diagonally in the 1.0 cm acrylic cuvettes filled with 3 ml basic medium at 37° C. Fura-2 fluorescence was measured with an Aminco-Bowman Series 2 luminescence spectrometer (SLM/Aminco, Urbana, Ill.) at excitation wavelengths of 340 and 380 nm and emission wavelength of 510 nm. Intracellular calcium was calculated from the 340/380 (nm) ratio using calibration curves and software. Stimulation of ECs with 1 U/ml thrombin (FIG. 34A) and 1 μM S1P (FIG. 34C) each induced intracellular calcium spikes. The addition of 8% HMW PEG caused a decrease in calcium signal (FIGS. 34B and 34D), in which pretreatment of HMW PEG blocked the S1P-induced calcium spike (FIG. 34D) but not the thrombin-induced calcium spike.

FIG. 35. Phorbol ester pretreatment attenuated barrier sustaining effects of HMW PEG. Endothelial cells were plated on gold microelectrodes and transendothelial electrical resistance (TEER), as measured in ohms, was measured to assess endothelial barrier permeability over a period of time. Confluent endothelium were either untreated or pretreated with phorbol ester (100 nM PMA, 24 hours) to down-regulate signaling molecules, such as Protein Kinase C, and subsequently challenged with 8% HMW PEG. Phorbol ester induced an increase in TEER that returned to a baseline of 1250 ohm after 8 hours. After 24 hours of PMA pretreatment, HMW PEG induced a potent increase in TEER similar to untreated cells, but the barrier enhancement was not sustained in the PMA-pretreated cells, consistent with the downregulation of key signaling proteins involved in maintaining endothelial barrier function upon HMW PEG challenge.

Exposure of cells to phorbol ester prior to exposure to HMW PEG showed an increased rate of reduction in TEER following the TEER spike associated with HMW PEG delivery. TEER was measured as a function of time for a period of 40 hours. Twenty-four hours after phorbol ester exposure at time t=0, cells were exposed to HMW PEG.

FIG. 36. The sustained barrier effect of high molecular weight PEG. HMW-PEG induced a potent and sustained, dose-dependent endothelial barrier enhancement that correlated with increased cortical actin reorganization.

FIG. 37. High molecular weight PEG blocked endothelial barrier dysfunction. HMW-PEG maintained endothelial barrier function above a basal level, thereby blocking a thrombin-induced barrier dysfunction.

FIG. 38. HMW PEG induces dose-dependent increases in endothelial barrier enhancement and alters F-actin redistribution. HLMVEC were plated on gold microelectrodes and grown to confluency. Changes in endothelial barrier permeability were assessed upon addition of 5-8% HMW PEG, in which increases in resistance correspond to enhanced barrier integrity. Inset are immunofluorescence images of unstimulated vs. HMW PEG-stimulated cells stained for F-actin. HMW PEG treatment (10%, 1 hour) resulted in increased cortical F-actin formation and bundling.

FIG. 39. HMW PEG inhibits ERK and MLC phosphorylation. S1P, a barrier-enhancing agent, has been shown to result in activation of specific signal transduction pathways as detected by phosphorylation of ERIC and MLC. Thus, the effects of HMW PEG at various concentration and treatment time on ERK and MLC phosphorylation is examined via Western blots. In contrast to S1P, HMW PEG potently inhibited basal phosphorylation of ERK (shown) and MLC.

FIG. 40. HMW PEG pretreatment protects cells from barrier disruption by blocking thrombin-induced ERK phosphorylation and/or by attenuating MLC phosphorylation. Cells were pretreated with HMW PEG (8%, 1 hr) and subsequently challenged with thrombin (1 U/ml). HMW PEG induced a rapid and sustained barrier enhancement which was able to provide barrier protection from thrombin by preventing a decrease in resistance below basal level. Since HMW PEG inhibited basal ERK and MLC phosphorylation, the ability of HMW PEG to inhibit thrombin-induced ERK and MLC activation was examined. Cells were treated with HMW PEG and challenged with thrombin (1 U/ml, 5 min). Lysates were processed for Western blot and probed for phospho-specific antibodies to ERK and MLC. HMW PEG completely blocked thrombin-induced ERK phosphorylation and potently attenuated thrombin-induced MLC phosphorylation.

FIG. 41. HMW PEG potently reversed thrombin-induced barrier disruption. Reversing barrier after disruption is clinically more-relevant than pretreatment with a drug. Thus, the ability of HMW PEG to reverse and enhance endothelial barrier function after thrombin challenge was examined. HPAEC were stimulated with thrombin (1 U/ml) for 20 minutes and subsequently treated with HMW PEG (8%) and compared with unstimulated cells or cells without thrombin stimulation. Thrombin induced a rapid and dynamic decrease in barrier resistance. Addition of HMW PEG at the maximum thrombin-induced decrease in resistance, resulted in a rapid barrier reversal and increased barrier resistance to levels of HMW PEG-treatment alone. Thus, HMW PEG reversed thrombin-induced barrier disruption and induced sustained barrier integrity after thrombin insult.

FIG. 42. HMW PEG potently reversed LPS-induced barrier disruption. Since thrombin is a rapid and temporal edemagenic agent, the effect of HMW PEG on a gradual barrier-inducing agent, LPS, was examined. HPAEC were stimulated with LPS (5 U/ml) for 4 hours and subsequently treated with HMW PEG (8%) and compared with unstimulated cells or cells without LPS stimulation. LPS induced a gradual and prolonged decrease in barrier resistance for over 20 hours. Addition of HMW PEG after 4 hours LPS challenge resulted in a rapid barrier reversal and increased barrier resistance near levels of HMW PEG-treatment alone. Similar to thrombin challenge, HMW PEG also reversed LPS-induced barrier disruption and induced sustained barrier integrity after LPS insult.

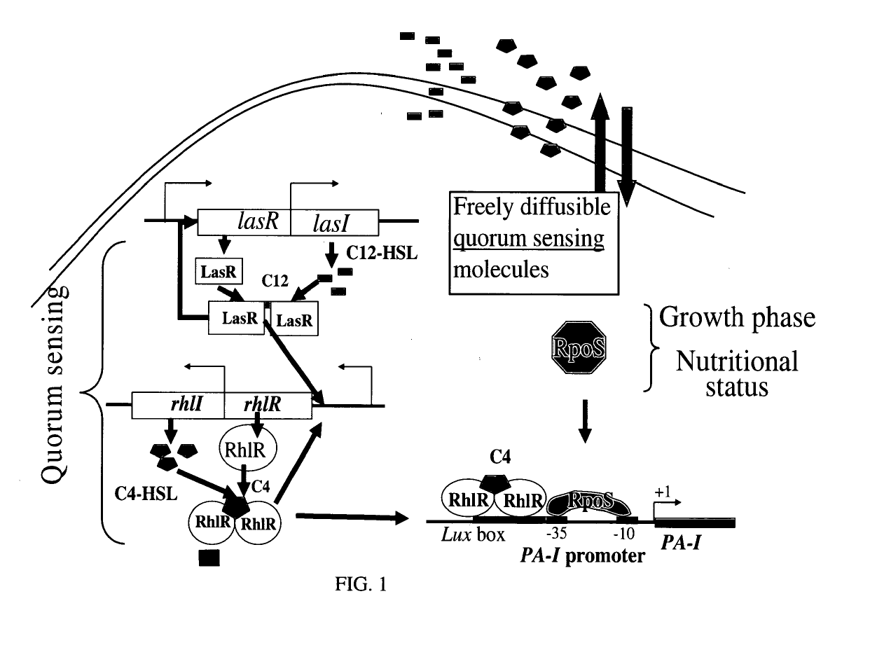

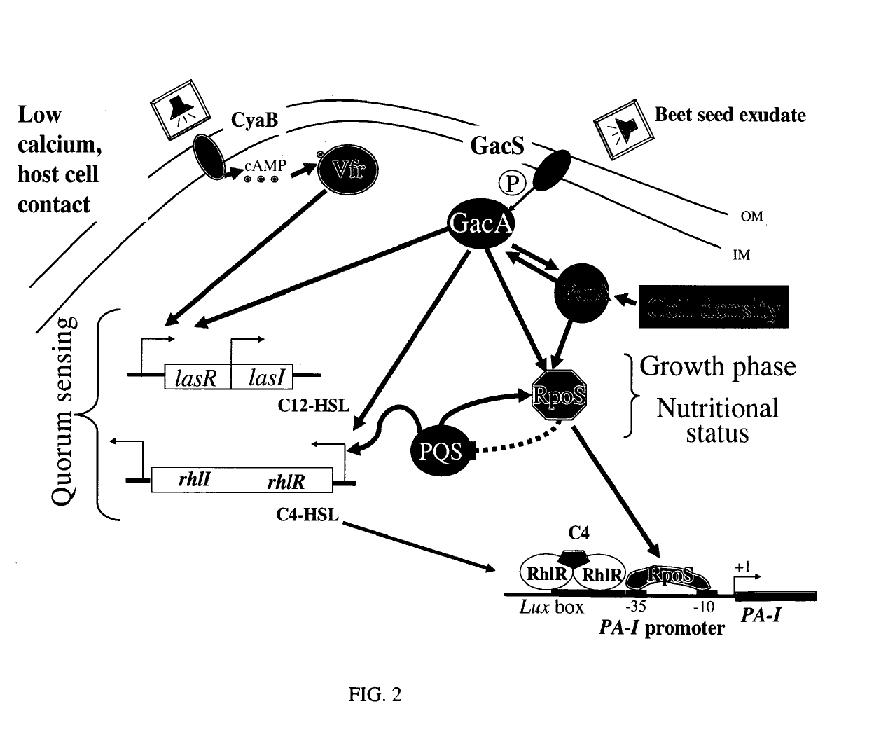

### DETAILED DESCRIPTION

Acute lung injury (ALI) and acute respiratory distress syndrome (ARDS) are inflammatory lung syndromes characterized by diffuse alveolar infiltration, hypoxemia, respiratory failure, and deaths due to multi-organ failure. Mortality rates in ARDS, the most severe ALI clinical scenario, range from 34 to 58% (MacCallum et al., Curr Opin Crit Care 11:43-49, 2005) with 150,000-200,000 ALI cases per year in the United States (Ware et al., N Engl J Med 342:1334-1349, 2000) and an incidence of 17-34 cases/100,000 people per year in Europe, Australia, and other developed countries. Thus ALI and ARDS constitute a major healthcare burden due to the intensive and often prolonged intensive care unit (ICU) hospitalizations. In addition to these epidemiologic studies, race and gender differences in ARDS deaths in the United States over the past several decades have demonstrated an increase in incidence and mortality due to sepsis and ALI in African-Americans when compared with Caucasians (Moss et al., Crit Care Med 30:1679 1685, 2002). ALI is usually caused by sepsis, acid inhalation, or trauma with mechanical ventilation, an intervention strategy commonly used in the ICU to treat ALI, potentially exacerbating ALI pathophysiology and reducing survival if excessive (Slutsky et al., Chest 116:9 S-15S, 1999, Tremblay et al., Crit Care Med 30:1693-1700, 2002, Kamat et al., Alcohol Clin Exp Res 29:1457-1465, 2005). The hallmarks for ALI, i.e., cellular and spatial heterogeneity, profound high permeability, leukocyte influx, and lung edema, are often augmented by mechanical ventilation in animal models of ALI (Zhang et al., Anesthesiology 97:1426-1433, 2002) and are contributing factors for death due to multiorgan failure (Slutsky et al., Am J Respir Crit Care Med 157:1721-1725, 1998).

Despite recent advances in low tidal volume mechanical ventilation and a better understanding of the underlying inflammatory pathophysiology of ALI, there remain few effective treatments for this devastating illness. ALI and the more severe acute respiratory distress syndrome (ARDS) represent a spectrum of common syndromes in response to a variety of infectious and noninfectious insults. The immune response to insults, such as infection or shock, includes recruitment of neutrophils and other inflammatory cells, induction of proinflammatory cytokines, and subsequent generation of reactive oxygen intermediates, which cause tissue damage and contribute to the induction and perpetuation of ALI (Lang et al., Chest 122:314 S-320S, 2002, 3). One consequence of enhanced oxidative stress is the peroxidation of phospholipids, abundantly present in the surfactant layer, into active metabolites, which have been observed in a variety of acute and chronic inflammatory diseases of the lung, including ARDS and asthma (Chabot et al., Eur Respir J 11:745-757. 1998, Wood et al., Eur Respir 721:177-186, 2003). In addition, reactive nitrogen species generated during acute inflammation may also modify blood-borne and cellular lipids, representing a novel source of bioactive oxidized or nitrated phospholipids (Pennathur et al., J Biol Chem 279:42977-42983, 2004, Kalyanaraman, Proc Natl Acad Sci (USA) 101:11527-11528, 2004). Increased production of exhaled isoprostanes serves as an index of oxidant stress and lipid oxidation in a number of lung pathologies (see Morrow et al., Am J Respir Crit Care Med 166:S25-S30, 2002, for review) and is elevated in ALI (Carpenter et al., Chest 114:1653-1659, 1998). After tissue insult, oxidized phospholipids released from membrane vesicles may serve as stress signals, triggering both pro- and anti-inflammatory cascades.

In the context of infectious injury to the lung, and other organs, identification of host stress bacterial signaling compounds and the bacterial membrane receptors to which they bind, will lead to the discovery of therapeutic targets that will allow for interdiction in the infectious process at its most proximate point. Furthermore, the identification of conserved bacterial receptors common to other microbial species will then lead to the development of receptor antagonists or decoys. Such an approach of rendering colonizing pathogens insensate to host stress activators, versus the current approach of elimination therapy with antibiotics, has the potential to provide an ecologically more neutral approach to infection prevention.

The ability of P. aeruginosa to be able to innocuously persist on mucosal surfaces one moment yet switch to a virulent phenotype the next, is one of the many characteristics that defines this highly opportunistic pathogen. In human disease, P. aeruginosa carries among the highest case-fatality rates of any hospital pathogen and is usually associated with patients exposed to prolonged and severe catabolic stress. In fact, among the most important predictors of mortality in patients infected with P. aeruginosa is the length and degree of physiologic stress and injury. The constancy of this observation led to the expectation that compounds released during host stress, especially those that characterize the most critically ill and injured patients, would serve as signals that directly activate the virulence machinery of P. aeruginosa.

While it is generally reasoned that lethal infections caused by opportunistic pathogens such as Pseudomonas are a result of a physiologically weakened host, an alternative explanation is that such pathogens have also evolved mechanisms to directly sense host stress and respond with enhanced virulence. Although it is well established that attachment and invasion of bacteria to host cells results in immediate recognition and immune activation, in the present study the possibility that within this interaction bacteria, in turn, recognize and respond to host immune activation, was considered.

The type I-P. aeruginosa lectin (PA-I or lecA gene product), an adhesin of P. aeruginosa, was used herein as a representative marker for virulence expression in this organism. The PA-I lectin (lecA) of P. aeruginosa was known to play a key role in the lethality of this organism in the intestinal tract of a stressed host by creating a permeability defect to its lethal cytotoxins. In addition, the PA-I lectin in P. aeruginosa is expressed in vivo within the intestinal tract of mice subjected to physiologic stress in the form of a 30% surgical hepatectomy. PA-I has been shown to also induce apoptosis in respiratory epithelial cells. Expression of PA-I (lecA) is regulated by three interconnected systems of virulence gene regulation, the quorum sensing signaling system (QS), the alternative sigma factor RpoS, and the Pseudomonas quinolone signaling system (PQS). Since the quorum sensing signaling system represents an important system of virulence gene regulation in P. aeruginosa, compounds released in response to host injury might signal P. aeruginosa via elements of the quorum sensing signaling system.

The opportunistic pathogen P. aeruginosa is able to sense the presence of the immune cytokine IFN-γ and respond by expressing two quorum sensing dependent virulence factors, PA-I and pyocyanin. From the perspective of P. aeruginosa, the ability to sense and respond to host immune activation, in particular to IFN-γ whose function is directed at bacterial clearance, provides this organism with a countermeasure against host immune activation. In particular, Interferon-γ is shown below to bind to an outer membrane protein in P. aeruginosa, OprF, resulting in the expression of a quorum sensing dependent virulence determinant, the PA-I lectin. These observations provide details of the mechanisms by which prokaryotic organisms are directly signaled by immune activation in their eukaryotic host.

Exposure of P. aeruginosa to opioids leads to the expression of several quorum sensing-dependent virulence factors in P. aeruginosa. That the QS system might be activated by opioids is a significant finding given that QS controls the expression of hundreds of virulence genes in P. aeruginosa.

Data disclosed herein provide evidence that MvfR is required for PCN production in response to U-50,488. In addition, data from these studies indicate that PCN production in response to U-50,488 also requires the synthesis of Pseudomonas quinolone signal (PQS), since methyl anthranilate attenuated the U-50,488-mediated effect on PCN production. That C4-HSL also requires intact MvfR to produce PCN, coupled with the finding of highly up-regulated PCN production in strains harboring multiple mvfR genes, is consistent with quorum sensing activation relying not only on the binding of QS signaling molecules to their core QS transcriptional regulators (i.e., RhlR, LasR), but also having QS signals activating proximal transcriptional regulators.

The data disclosed herein establish that opioid compounds may vary in their ability to induce a particular virulence phenotype in P. aeruginosa. It is contemplated that there are multiple host-stress-derived bacterial signaling compounds that are able to influence the state of virulence in P. aeruginosa. Norepinephrine can also affect the QS-dependent virulence factor PA-IL in P. aeruginosa and soluble compounds released into the media by hypoxic intestinal epithelial cells also induce PA-IL expression. Consistent with these disclosures is the knowledge that norepinephrine directly affects QS circuitry in E.

Identifying the host stress-derived compounds to which colonizing bacteria respond with enhanced virulence has led to the development of strategies to render bacteria insensate to host stress. The design of specific compounds that interdict in the lines of communication between host factors and the bacterial sensor mechanisms that recognize these factors, has led to strategies which seek to contain rather than eliminate opportunistic pathogens that typically colonize the epithelium or endothelium of a stressed host.

An understanding of the following more detailed description of the invention is facilitated by establishing the following meanings for terms used in this disclosure, and by a consideration of co-owned International Patent Application No. PCT/US02/37498, filed Nov. 26, 2002 and provisional U.S. Patent Application No. 60/564,031, filed Apr. 20, 2004. Each of these applications is incorporated herein in its entirety.

An “abnormal condition” is broadly defined to include mammalian diseases, mammalian disorders and any abnormal state of mammalian health that is characterized by an epithelial surface at risk of developing a microbial-mediated disorder. The abnormal conditions characterized by an epithelial surface at risk of developing a microbial-mediated disorder include conditions in which the epithelial surface has developed a microbial-mediated disorder. Exemplary conditions include human diseases and human disorders requiring, or resulting from, medical intervention, such as a burn injury, neonatal enterocolitis, severe neutropenia, inflammatory bowel disease, enteropathy (e.g., of the critically ill) and transplant (e.g., organ) rejection.

“Burn injury” means damage to mammalian tissue resulting from exposure of the tissue to heat, for example in the form of an open flame, steam, hot fluid, and a hot surface.

A “chemical contact” injury refers to an injury caused by direct contact with a chemical and can involve a chemical burn or other injury.

“Severe” neutropenia is given its ordinary and accustomed meaning of a marked decrease in the number of circulating neutrophils.

“Transplant rejection” refers to any development of transplanted material (e.g., an organ) recognized as being associated with ultimate rejection of that material by the host organism.

“Administering” is given its ordinary and accustomed meaning of delivery by any suitable means recognized in the art. Exemplary forms of administering include oral delivery, anal delivery, direct puncture or injection, including intravenous, intraperitoneal, intramuscular, subcutaneous, and other forms of injection, topical application, and spray (e.g., nebulizing spray), gel or fluid application to an eye, ear, nose, mouth, anus or urethral opening, and cannulation.

An “effective dose” is that amount of a substance that provides a beneficial effect on the organism receiving the dose and may vary depending upon the purpose of administering the dose, the size and condition of the organism receiving the dose, and other variables recognized in the art as relevant to a determination of an effective does. The process of determining an effective dose involves routine optimization procedures that are within the skill in the art.

An “animal” is given its conventional meaning of a non-plant, non-protist living being. A preferred animal is a mammal, such as a human.

In the context of the present disclosure, a “need” is an organismal, organ, tissue, or cellular state that could benefit from administration of an effective dose to an organism characterized by that state. For example, a human at risk of developing gut-derived sepsis, or presenting a symptom thereof, is an organism in need of an effective dose of a product, such as a pharmaceutical composition, according to the present invention.

“Average molecular weight” is given its ordinary and accustomed meaning of the arithmetic mean of the molecular weights of the components (e.g., molecules) of a composition, regardless of the accuracy of the determination of that mean. For example, polyethylene glycol, or PEG, having an average molecular weight of 3.5 kilodaltons may contain PEG molecules of varying molecular weight, provided that the arithmetic mean of those molecular weights is determined to be 3.5 kilodaltons at some level of accuracy, which may reflect an estimate of the arithmetic mean, as would be understood in the art. Analogously, PEG 15-20 means PEG whose molecular weights yield an arithmetic mean between 15 and 20 kilodaltons, with that arithmetic mean subject to the caveats noted above. These PEG molecules include, but are not limited to, simple PEG polymers. For example, a plurality of relatively smaller PEG molecules (e.g., 7,000 to 10,000 daltons) may be joined, optionally with a linker molecule such as a phenol, into a single molecule having a higher average molecular weight (e.g., 15,000 to 20,000 daltons).

“Cell membrane integrity” means the relative absence of functionally significant modifications of a cell membrane as a functional component of a living cell, as would be understood in the art.

“Detectably altered” is given its ordinary and accustomed meaning of a change that is perceivable using detection means suitable under the circumstances, as would be understood in the art.

“Growth pattern” refers collectively to the values of those properties of a cell, or group of cells (e.g., a population of cells), that are recognized in the art as characterizing cell growth, such as the generation or doubling time of the cell, the appearance of topography of a nascent group of cells, and other variables recognized in the art as contributing to an understanding of the growth pattern of a cell or group of cells.

“Inhibiting” is given its ordinary and accustomed meaning of inhibiting with, reducing or preventing. For example, inhibiting morphological change means that morphological change is made more difficult or prevented entirely.

“PA-I,” or “PA-I lectin/adhesin,” or “PA-IL” expression means the production or generation of an activity characteristic of PA-I lectin/adhesin. Typically, PA-I lectin/adhesin expression involves translation of a PA-I lectin/adhesin-encoding mRNA to yield a PA-I lectin/adhesin polypeptide having at least one activity characteristic of PA-I lectin/adhesin. Optionally, PA-I lectin/adhesin further includes transcription of a PA-I lectin/adhesin-encoding DNA to yield the aforementioned mRNA.

“Epithelium-induced activation” and “endothelium-induced activation” refer to an increase in the activity of a given target (e.g., PA-I lectin/adhesin) through direct or indirect influence of an epithelial or endothelial cell, respectively. In the context of the present invention, for example, endothelium-induced activation of PA-I Iectin/adhesin refers to an increase in that polypeptide's activity attributable to the indirect influence of an endothelium manifested through the direct contact of an endothelial cell or cells with a microbial pathogen.

“Morphological change” is given its ordinary and accustomed meaning of an alteration in form.

“Intestinal pathogen” means a microbial pathogen capable of causing, in whole or part, gut-derived sepsis in an animal such as a human. Analogously, “pulmonary pathogen” means a microbial pathogen capable of causing, in whole or part, a lung disorder in an animal such as a human. Intestinal and pulmonary pathogens known in the art are embraced by this definition, including gram negative bacilli such as the Pseudomonads (e.g., Pseudomonas aeruginosa).

“Ameliorating” means reducing the degree or severity of, consistent with its ordinary and accustomed meaning.

“Pathogenic quorum” means aggregation or association of a sufficient number of pathogenic organisms (e.g., P. aeruginosa) to initiate or maintain a quorum sensing signal or communication that a threshold concentration, or number, of organisms (e.g., intestinal pathogens) are present, as would be known in the art.

“Interaction” is given its ordinary and accustomed meaning of interplay, as in the interplay between or among two or more biological products, such as molecules, cells, and the like.

“Transepithelial Electrical Resistance,” or TEER, is given the meaning this phrase has acquired in the art, which refers to a measurement of electrical resistance across epithelial tissue, which is non-exclusively useful in assessing the status of tight junctions between epithelial cells in an epithelial tissue. “TEER” is also used to refer to transendothelial electrical resistance, with the intended meaning of TEER apparent from the context of its usage.

“Adherence” is given its ordinary and accustomed meaning of physically associating for longer than a transient period of time.

“Topographically asymmetrical” refers to an image, map or other representation of the surface of a three-dimensional object (e.g., a cell) that is not symmetrical.

“Atomic force microscopy,” also known as scanning force microscopy, is a technique for acquiring a high-resolution topographical map of a substance by having a cantilevered probe traverse the surface of a sample in a raster scan and using highly sensitive means for detecting probe deflections, as would be understood in the art.

“Pharmaceutical composition” means a formulation of compounds suitable for therapeutic administration, to a living animal, such as a human patient. Preferred pharmaceutical compositions according to the invention comprise a solution balanced in viscosity, electrolyte profile and osmolality, comprising an electrolyte, dextran-coated L-glutamine, dextran-coated inulin, lactulase, D-galactose, N-acetyl D-galactosamine and 5-20% HMW PEG (e.g., 15,000-20,000 daltons; see below).

“Adjuvants,” “carriers,” or “diluents” are each given the meanings those terms have acquired in the art. An adjuvant is one or more substances that serve to prolong the immunogenicity of a co-administered immunogen. A carrier is one or more substances that facilitate the manipulation, such as by translocation of a substance being canned. A diluent is one or more substances that reduce the concentration of, or dilute, a given substance exposed to the diluent.

“HMW PEG-like compounds” refer to relatively high molecular weight PEG compounds, defined as having an average molecular weight greater than 3.5 kilodaltons (kD). Preferably, HMW PEG has an average molecular weight greater than 5 kilodaltons and, in particular embodiments, HMW PEG has an average molecular weight at least 8 kilodaltons, more than 12 kilodaltons, at least 15 kilodaltons, and between 15 and 20 kilodaltons. Additionally, “HMW PEG-like compounds includes HMW PEG derivatives wherein each such derivative is an HMW PEG containing at least one additional functional group. Preferred HMW PEG derivatives are cationic polymers. Exemplary functional groups include any of the alkoxy series, preferably C1-C10, any of the aryloxy series, phenyl and substituted phenyl groups. Such functional groups may be attached at any point to an HMW PEG molecule, including at either terminus or in the middle; also included are functional groups, e.g., phenyl and its substituents, that serve to link to smaller PEG molecules or derivative thereof into a single HMW PEG-like compound. Further, the HMW PEG-like molecules having an additional functional group may have one such group or more than one such group; each molecule may also have a mixture of additional functional groups, provided such molecules are useful in stabilizing at least one therapeutic during delivery thereof or in treating, ameliorating or preventing a disease, disorder or condition of an epithelial cell.

“Media” and “medium” are used to refer to cell culture medium and to cell culture media throughout the application. The singular or plural number of the nouns will be apparent from context in each usage.

“Associated” in the context of a paracellular barrier dysfunction being associated with a lung disorder means that the dysfunction is observed in an organism suffering from a lung disorder, such as pulmonary edema, and does not imply any causal relationship between the paracellular hairier dysfunction and the lung disorder.

In general terms, a model of lethal sepsis in mice has been developed which provides unique insight into the process by which microbial pathogens can cause lethal sepsis syndrome from within the intestinal tract of a physiologically stressed host. As in human disease, three physiologic “hits” result in mortality, e.g., surgical stress (30% hepatectomy), starvation (48 hour of water only) and the introduction of P. aeruginosa into the distal intestinal tract (cecum). This model results in 100% mortality, whereas elimination of any one of the three factors results in complete survival. A single virulence determinant has been identified in Pseudomonas aeruginosa, PA-I, that is expressed in vivo in response to locally released compounds unique to the intestinal tract of a physiologically stressed host. That PA-I plays a role in lethal gut-derived sepsis, such as in mice, was demonstrated by experiments in which mutagenized strains of P. aeruginosa, void of PA-I yet capable of secreting exotoxin A, had markedly attenuated effects on the barrier function of cultured epithelial cells and were completely apathogenic in the mouse model of lethal gut-derived sepsis. PA-I lectin/adhesin plays a key role in the lethal effect of this organism by creating a permeability defect to potent and lethal cytotoxins of P. aeruginosa, such as exotoxin A and elastase. The lethal effect of intestinal P. aeruginosa appears to occur completely independent of its extraintestinal dissemination (translocation). Surprisingly, systemic injection (intravenous, intraperitoneal) of an equal dose of P. aeruginosa in this model produces no mortality and no systemic inflammation. Taken together, the data provide strong evidence that sepsis can be generated by a microbial pathogen whose virulence is activated locally by host stress-derived BSCs generated during surgical stress.

Observation that P. aeruginosa is much more virulent and lethal when present on an epithelial, or endothelial, surface than when bloodborne is supported by a lung model of sepsis (Kurahashi et al., J Clin Invest, 104:743-50, 1999). Intravenous injection of a highly cytotoxic strain of P. aeruginosa, PA 103, resulted in no systemic cytokine release and no mortality in rabbits, whereas lung instillation of an equal dose (approximately 10⁸ cfu/ml) resulted in significant systemic cytokine release (TNFα, IL-8) and 100% mortality. An extensive number of studies have now demonstrated that the most virulent and lethal strains of P. aeruginosa causing sepsis following lung instillation are not those that display the most invasive (translocating/disseminating) phenotype, but rather are those strains that are most disruptive of cellular integrity and epithelial permselectivity to its locally released cytotoxins. These observations, coupled with the findings that P. aeruginosa produces a 25-fold increase in its extracellular virulence factors (i.e., elastase, alkaline protease) when cultured in the presence of epithelial cells, suggests that the lethality of this pathogen is governed by its interaction with, and activation by, the epithelium itself. Experimental data show that both soluble and contact-mediated elements of the intestinal epithelium exposed to stress, enhance the capacity of P. aeruginosa to express PA-I, which is capable of causing a profound disruption in the cellular integrity of both intestinal and lung epithelial cells. Data disclosed herein extends these observations to endothelial cells of the lung.

The gene encoding PA-I (the lecA gene) is an ideal biological “read-out” and reporter gene in which to examine overall virulence gene expression in P. aeruginosa in response to host stress-derived BSCs.

The precise host cell elements that activate bacterial biosensors are not known. Because PA-I expression is both QS and RpoS dependent, GFP-PA-I reporter strains provide a unique opportunity to screen for host cell-derived bacterial signaling compounds released during stress that activate membrane sensors, leading to PA-I expression.

Various opioid receptor agonists, including endogenous morphine alkaloids, are released and maintained at sustained concentrations during severe stress. Opioids are highly conserved compounds and various bacteria and fungi, including P. aeruginosa, synthesize and metabolize morphine. Similarly, as shown herein, elements of the immune system, such as IFN-γ, can also serve as potent host stress-derived BSCs. Although there is data demonstrating that bacteria can activate multiple elements of the host immune system via several bacterial-derived compounds, that such a chemical dialogue functions bidirectionally is virtually unexplored. The ability to expose whole animal models and cell culture systems to clinically relevant stress, while harvesting extracellular fluids and media that can be screened for the presence of host cell-derived bacterial signaling compounds using GPF-PA-I reporter strains, fills a critical gap in knowledge that exists in the area of microbial pathogenesis. Such an approach opens new therapeutic targets for interdiction in the infectious process at its most proximate point.

The therapeutics according to the invention comprise a HMW PEG-like compound, which may be administered by any means suitable for the condition or disorder to be treated. The compound(s) may be delivered orally, such as in the form of tablets, capsules, granules, powders, or with liquid formulations including syrups; by sublingual; buccal; or transdermal delivery; by injection or infusion parenterally, subcutaneously, intravenously, intramuscularly, or intrasternally (e.g., as sterile injectable aqueous or non-aqueous solutions or suspensions); nasally, such as by inhalation spray; rectally such as in the form of suppositories; vaginally or urethrally via suppository or infusion, e.g., via cannulation, or liposomally. Dosage unit formulations containing non-toxic, pharmaceutically acceptable vehicles or diluents may be administered. The compounds may be administered in a form suitable for immediate release or extended release. Immediate release or extended release may be achieved with suitable pharmaceutical compositions known in the art.

Exemplary compositions for oral administration include suspensions which may contain, for example, microcrystalline cellulose for imparting bulk, alginic acid or sodium alginate as a suspending agent, methylcellulose as a viscosity enhancer, sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrates, diluents and lubricants, such as those known in the art. The inventive compounds may be orally delivered by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be excipients such as a relatively high molecular weight cellulose (AVICEL®) or a polyethylene glycol (PEG; GoLytely®, 3.34 kD); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®). Lubricants, glidants, flavors, coloring agents and stabilizers may also be added for ease of fabrication and use.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

The effective amount of a compound of the present invention may be determined by one of ordinary skill in the art. The specific dose level and frequency of dosage for any particular subject may vary and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, horses, and the like, at risk of developing a microbe-mediated epithelial condition or disease, such as gut-derived sepsis.

The data disclosed herein (see Examples) establish the following:

First, opioids, Interferon-gamma, and media from Caco-2 cells exposed to ischemia and hyperthermia, potently activate the virulence of P. aeruginosa as judged by the expression of PA-I and the ability to shift the barrier dysregulating dynamics of P. aeruginosa against Caco-2 cells.

Second, in clinical models of surgical stress (hepatectomy, ischemia/reperfusion injury to the gut), soluble factors are released into the gut that induce a robust PA-I response in P. aeruginosa.

Third, strains of P. aeruginosa harvested from critically ill humans have a high prevalence of the PA-I genotype/phenotype.

Fourth, strains of P. aeruginosa harvested from critically ill humans display a high level of aggressiveness (adherence, alteration in barrier function, cytotoxicity) to cultured epithelial cells compared to strains harvested from hospitalized non-critically ill patients.

And fifth, high molecular weight polymers (15-20,00 dalton HMW PEG) can render P. aeruginosa completely insensate to host stress-derived bacterial signaling compounds and protect mice from pathogenesis.

### EXAMPLES

### Example 1

### Construction of GFP-PA-I Reporter Strains

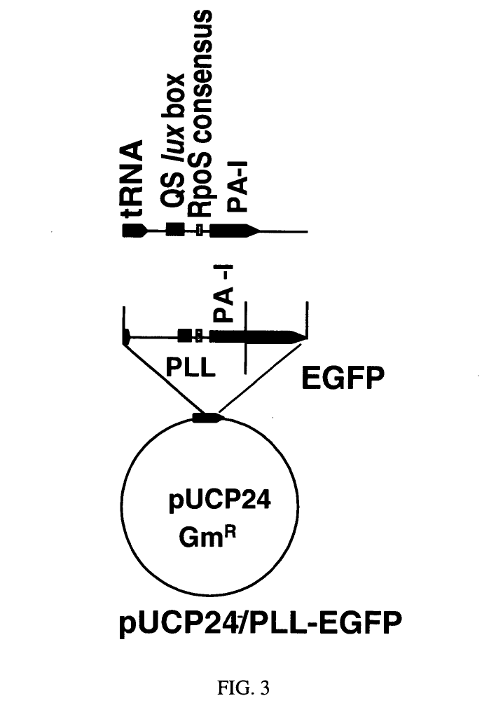

A plasmid containing the GFP-PA-I fusion construct was constructed using conventional recombinant DNA techniques. The EGFP gene encoding green fluorescent protein was amplified using the pBI-EGFP plasmid (Clontech) as a template. XbaI and PstI restriction sites were introduced using primers TCTAGAACTAGTGGATCCCCGCGGATG (SEQ ID NO: 1) and GCAGACTAGGTCGACAAGCTTGATATC (SEQ ID NO: 2). The PGR product was cloned directly into the pCR 2.1 vector using a TA-cloning kit (Invitrogen), followed by transformation of the pCR2.1/EGFP construct into E. coli DH5a. The EGFP gene was excised from this construct by digestion with XbaI and PstI and the fragment containing the excised gene was cloned into the E. coli-P. aeruginosa shuttle vector pUCP24, which had been digested with the same restriction enzymes. The resulting construct (i.e., pUCP24/EGFP), containing the EGFP gene in the shuttle vector, was typically electroporated at 25 μF and 2500 V into P. Aeruginosa electrocompetent cells. Cells containing pUCP24/EGFP were selected by gentamicin (Gm)challenge, typically at 100 μg/ml. As illustrated in FIG. 3, a derivative of pUCP24/EGFP was generated that placed the PA-I lectin/adhesin gene in close proximity to the EGFP gene, effectively linking the genes genetically. In addition to incorporating the structural lecA gene, the construct contained the QS lux box and RpoS consensus sequences in the 5′ non-coding region of lecA, along with rRNA sequence. The derivative construct was termed pUCP24/PLL-EGFP.

Upon consideration of FIG. 3, in conjunction with the explanatory text provided herein, one of skill would understand how to make and use the above-described construct, as well as other suitable constructs for providing lecA, alone or in physical proximity to a marker gene such as EGFP, using any of a variety of techniques.

### Example 2

### Location on of PA-I

PA-I lectin/adhesin was localized to a previously undescribed structural appendage on the outer surface of P. aeruginosa, using conventional techniques as would be understood in the art.

### Example 3

### Correlation of In Vitro and In Vivo Observations

C. elegans is suitable as an in vivo model system for BSC signaling and its role in the production of PA-I, C. elegans is accepted as a highly accurate and predictable model in which to study the host response to P. aeruginosa (Aballay et al., Curr Opin Microbiol, 5:97-101, 2002; Tan et al., Proc Natl Acad Sci (USA) 96:715-20, 1999). C. elegans worms feed on lawns of P. aeruginosa growing on solid agar and, thus, provides an ideal system in which to study microbial pathogenesis, especially in regard to gut-derived sepsis, since the mode of infectivity is via the digestive tract. These nematodes readily feed on bacteria such as E. coli growing on solid agar plates, yet when fed specific strains of P. aeruginosa, mortality rates exceed 50% within 72 hours. Mortality rates with this model have been shown to be dependent on both the agar environment as well as the strain of P. aeruginosa. Certain strains are highly lethal in this model (e.g., PAH), whereas other strains (PAO1) show intermediate kill rates. The ability to feed C. elegans on lawns of the completely sequenced P. aeruginosa strain PAO1, and selected transposon mutants, while enriching agar plates with various host stress-derived BSCs screened for their ability to express PA-I, makes available a rapid screening system for genes that actively participate in in vivo virulence against the intestinal epithelium. With this approach, the virulence phenotype observed in vitro has been transferred to an in vivo model, with the expectation that results obtained with such a model will prove much more reliable in accurately characterizing the virulence phenotype observed in human patients suffering from an epithelial cell barrier dysfunction. Moreover, it is expected that such results will provide reasonable accuracy in characterizing the virulence phenotype observed in patients suffering from cell barrier dysfunctions in general, such as endothelial cell barrier dysfunction (e.g., lung endothelial cell dysfunction) as well as epithelial cell barrier dysfunction.

### Example 4

### In Vitro Recapitulation of the In Vivo “Cues” Released During Surgical Stress

In vitro studies demonstrated that pH, osmolality, and norepinephrine did not change PA-I expression, while opioids, interferon-gamma, C4-HSL, and media from hypoxic and hyperthermic intestinal epithelial cells induced PA-I expression. It is expected that hypoxic and hyperthermic lung epithelial and lung endothelial cells will also induce PA-I expression. PA-I was functionally expressed in cell assays in the presence of the PA-I-inducing compounds.

### Example 5

### Toxin Flux Across Epithelia

Exotoxin A was labeled with AlexaFluor 594, and its transepithelial flux was measured at varying levels of decrease of transepithelial resistance (TEER) of MDCK monolayers that was achieved by apical application of MDCK cells to different concentrations of pure PA-I protein. A five-fold increase in exotoxin A flux across MDCK cells was found when transepithelial resistance was decreased below 50% of control. Purified PA-I decreased the TEER of epithelial cells to the same degree as P. aeruginosa. PA-I null mutants of P. aeruginosa had a significantly attenuated effect on the transepithelial resistance of MDCK cells. Techniques used in conducting the experiments are described in Example 24, below, or are conventional in the art. Apparent from this disclosure is that PA-I expression by a Pseudomonad such as P. aeruginosa will lead to decreases in transcellular resistance, whether the cell-cell barrier is provided by epithelial or endothelial cells and irrespective of the organ and/or tissue type of those cells.

### Example 6

### Response of Cells to Purified PA-I

The degree of cell polarity (i.e., degree of cell confluency and tight junctional apposition) has been shown to dictate the degree of response to purified PA-I protein. Cells that were loosely confluent had a more profound fall in TEER in response to PA-I compared to “tighter” and more differentiated cell monolayers. In addition, wounded monolayers exposed dense areas of PA-I binding. Cell culturing was performed as described in Example 24, below; relative confluency was assessed using conventional techniques as would be known in the art.

### Example 7

### Soluble Host Factors Induce Expression of PA-I Lectin/Adhesin

GFP-reporter strains permit demonstrations that virulence gene expression in P. aeruginosa is expressed in vivo within the intestinal tract of a stressed (30% hepatectomy) host. EGFP reporter constructs were specifically designed to contain known upstream regulatory regions involved in PA-I expression (e.g., lux box (QS promoter elements) and RpoS; see FIG. 3). The EGFP-PA-I reporter strain, termed PLL-EGFP, was then injected into the cecum of sham-operated (control) mice and mice undergoing surgical hepatectomy. Twenty-four hours later, feces and washed cecal mucosa were then assayed for the presence of fluorescent bacteria. Both within the cecal lumen and in response to contact with the intestinal epithelium, PA-I is expressed in vivo in response to elements of the local intestinal microenvironment (cecum) of mice subjected to catabolic (surgical) stress. These findings were verified in the non-reporter strain, PA27853, using an assay in which bacterial RNA is extracted from fresh feces using a RNA protection system. Reiterative studies were performed in which PA27853 was introduced into the cecum of control and hepatectomized mice and then bacterial RNA recovered from fresh feces 24 hours later for quantitative RT-PCR (QRT-PCR) of both PA-I and exotoxin A. This assay provides a precise molecular “snapshot” of the effect of the in situ cecal environment on P. aeruginosa virulence gene expression. Results demonstrated that the cecal microenvironment of a stressed host induced PA-I and exotoxin A virulence gene expression. Next, in order to determine whether these findings were due to soluble factors released into the intestinal lumen, particulate-free filtrates were prepared from cecal luminal contents from control and hepatectomized mice and added to fresh cultures of the reporter strain PLL-EGFP. Results demonstrated that when PA-I GFP reporter strains were exposed to filtered cecal contents from mice exposed to surgical hepatectomy, a 248%±12 increase in fluorescence was observed compared to 112%±15 for filtered cecal contents from sham-operated mice (P<0.001). These results indicated that a soluble factor is present in the intestinal lumen following surgical stress that activates PA-I expression. Two remaining issues included, first, whether the soluble PA-I-inducing components are generated from within the intestinal tract itself or from the systemic compartment and, second, whether the soluble PA-I-inducing components are specific to hepatectomy-induced stress. To address these issues an animal model of segmental intestinal ischemia was developed in which an isolated loop of intestine (6 cm, proximal ileum) was luminally cannulated and timed aliquots of luminal perfusates were collected following 10 minutes of ischemia followed by 10 minutes of reperfusion. Blood was then obtained at the end of the experiment in order to determine the effect of systemic factors on PA-I expression. Results indicated that 1) intestinal ischemia, similar to hepatectomy, can release soluble factors into the intestinal lumen capable of signaling P. aeruginosa to express PA-I; 2) these factors may originate from the intestinal tract itself, since during ischemia the intestine is isolated from systemic factors; 3) blood components do not induce PA-I expression; and 4) the presence of the normal flora, virtually absent in flushed small bowel segments, appears to play no role in this response. To rule out the possibility that the in vivo expression of PA-I was due to secondary effects of surgical stress on physico-chemical changes in the local microenvironment, P. aeruginosa strain PA-27853 and reporter strains (PLL-EGFP) were exposed to ambient hypoxia (0.3% O₂), pH changes (6-8), and 80% CO₂. None of these conditions induced PA-I expression. In addition, reporter strains exposed to the blood or liver tissue of mice following sham-operation or hepatectomy, did not display enhanced fluorescence. These studies suggest that bacterial signaling components released in response to surgical and ischemic stress are highly concentrated in the intestinal tract and are generated by host-cell derived factors that can be isolated from, and detected within, the intestinal lumen. Based on these results, it is expected that any form of stress (e.g., surgery, injury such as traumatic injury, illness, heat, starvation, hypoxia, and the like) to cells, such as intestinal epithelial cells, lung epithelial cells and lung endothelial cells (i.e., any cells engaged in cell-cell interactions in the proximity of a potential microbial pathogen such as a Pseudomonad) will typically lead to a change in the level of at least one soluble factor involved in bacterial signaling, i.e., at least one soluble BSC.

### Example 8

### Identification of Host Stress-Derived BSCs by Screening Candidate Agents: The Role of Cytokines

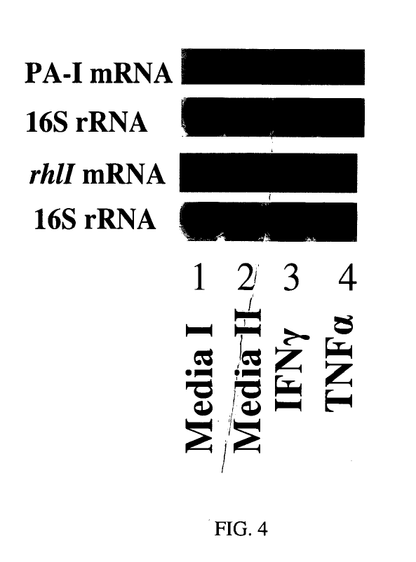

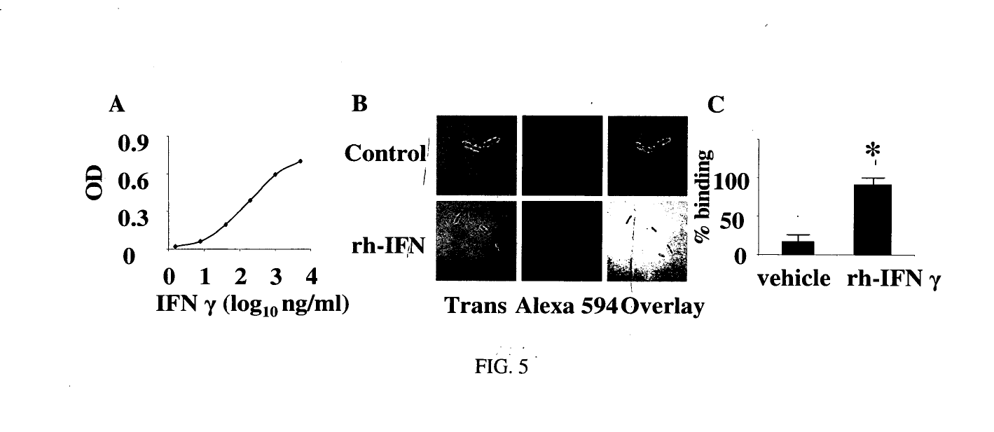

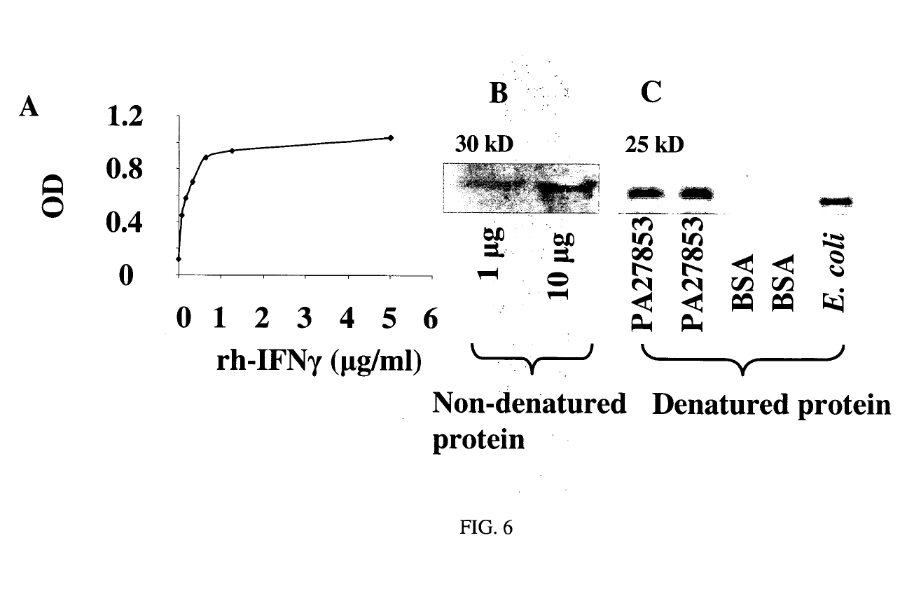

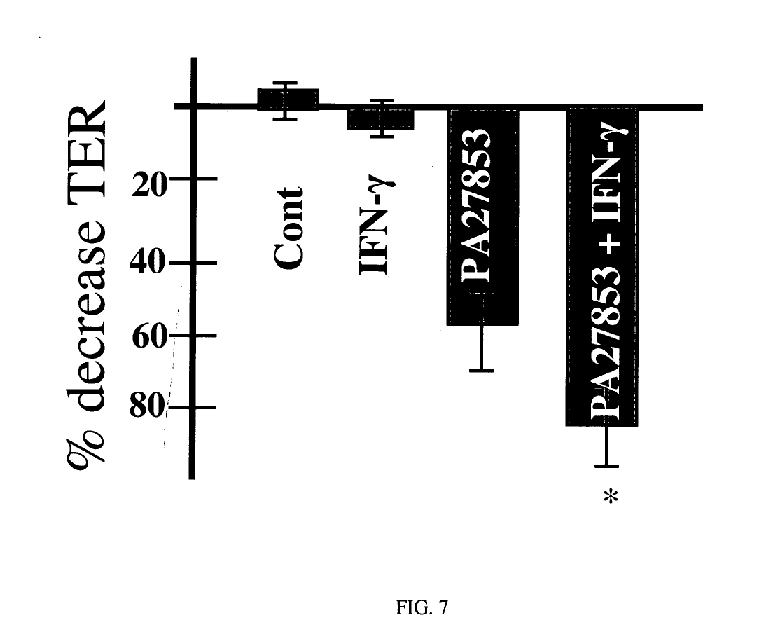

As a method to rapidly identify host BSCs, P. aeruginosa strains were exposed to media containing adenosine (released by Caco-2 cells in response to hypoxia) TNFα, IL-2, IL-6 IL-8 (released by epithelia in response to bacterial invasion/ischemia), and IFNγ (released by intraepithelial lymphocytes in response to bacterial invasion/ischemia). In addition, strains were exposed to apical media from Caco-2 cells basolaterally exposed to one or more of the various epithelial-derived cytokines. Basolateral exposure of Caco-2 cells to the combination of IFN γ and TNF α activates cellular signaling proteins that dramatically alter tight junctional proteins and function. Media from Caco-2 cells exposed to various combinations of these cytokines had no effect on PA-I expression. However, IFN-γ alone induced a direct effect on PA-I expression while none of the other compounds alone had any effect (FIG. 4). Another issue was whether IFN γ binding to P. aeruginosa could be demonstrated for strain PA27853. Using ELISA, immunofluorescence microscopy, and flow cytometry, the binding characteristics of IFNγ were determined for both whole bacteria and membrane fractions of P. aeruginosa. Results demonstrated that IFN-γ showed high binding affinity to whole bacterial cells of PA27853 (FIG. 5). These effects were also observed with strain PAO1. Next, solubilized and separated membrane proteins of P. aeruginosa (PA27853) were solubilized and separated, which showed that IFN-γ avidly binds to a single 30 kDa protein band (FIG. 6). This protein can also be immunoprecipitated from E. coli (FIG. 6C). Next, IFN-γ binding specificity to whole bacterial cells was determined using reiterative binding studies in the presence of various gram-negative bacterial strains, including P. aeruginosa. Multiple strains of bacteria displayed IFN-γ binding by ELISA binding assays, indicating that an IFN-γ binding site may be conserved across a wide variety of prokaryotic cells. Finally, in order to determine if PA-I was functionally expressed in PA27853 in the presence IFN-γ, PA27853 was inoculated onto Caco-2 cell monolayers in the presence of IFN-γ and the effect on barrier dysregulating dynamics of PA27853 against this cell line were assessed to determine if IFN-γ shifted the dynamics. Results seen in FIG. 7 demonstrate that IFN-γ enhanced the barrier dysregulating effect of PA27853 against the intestinal epithelium after five hours of incubation. Thus, cytokines such as IFN-γ are embraced by the invention as effective modulators of bacterial signaling and, ultimately, of eukaryotic (e.g., epithelial, endothelial) cell barrier function.

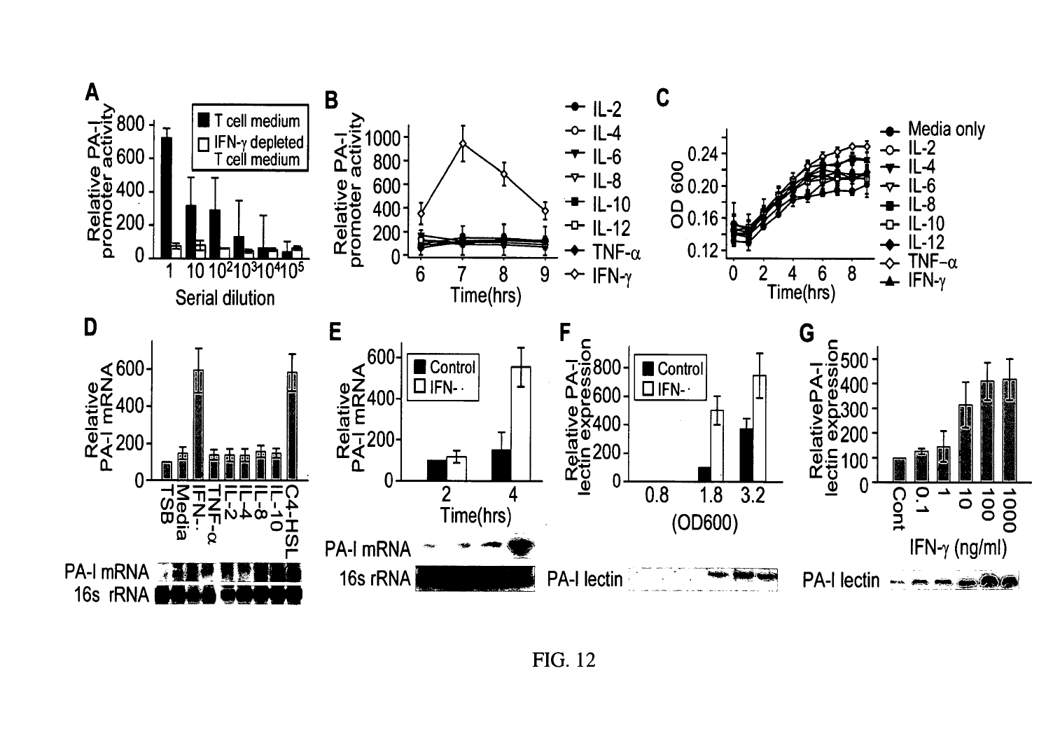

The expression of virulence in P. aeruginosa is highly regulated by the quorum sensing signaling system (QS), a hierarchical system of virulence gene regulation that is dependent on bacterial cell density and hence growth phase. In order to determine the effect of growth phase on the response of P. aeruginosa to IFN-γ, bacteria were harvested at various growth phases following exposure to IFN-γ, and PA-I mRNA and protein were measured by Northern blot and immunoblot, respectively. Both PA-I mRNA and protein were increased in response to IFN-γ at early stationary phase of growth (FIGS. 12E, 12F). PA-I protein expression in PAO1 was also dose dependent, with the greatest increase seen with 100 ng/ml (FIG. 12G). Taken together, these results indicated that the exposure of P. aeruginosa to IFN-γ enhanced PA-I expression but was not able to shift its expression to an earlier phase of growth.

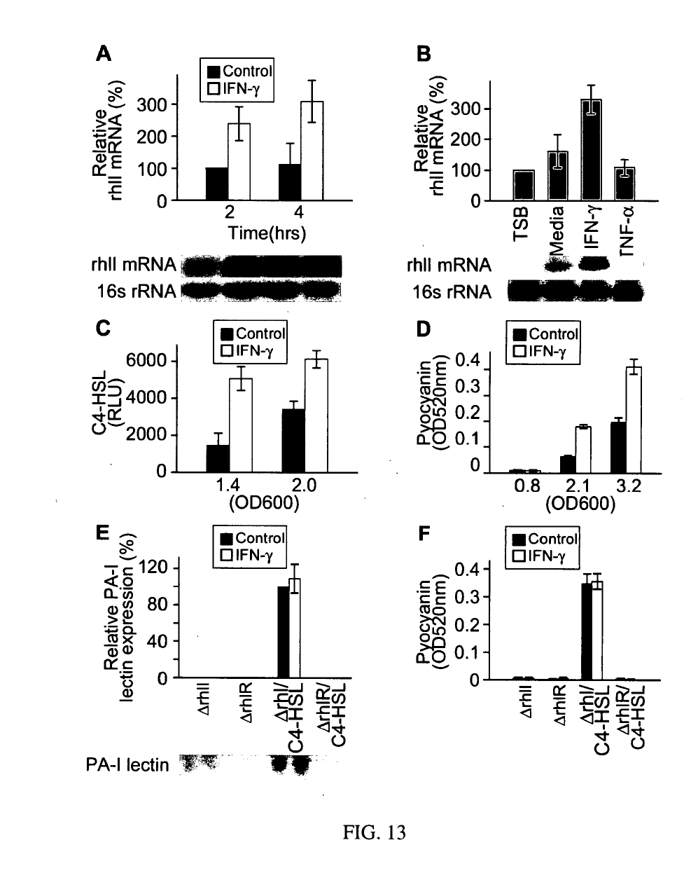

To determine whether IFN-γ induced PA-I via activation of the quorum sensing signaling system, rhlI gene expression in PAO1 in response to IFN-γ was measured by Northern blot. IFN-γ induced rhlI transcription in PAO1 (FIGS. 13A, 13B). RhlI is the gene required for the synthesis of C₄-HSL (C₄-homoserine lactone), a core quorum sensing signaling molecule that plays a key role in the expression of PA-I. Next, the exposure of P. aeruginosa to IFN-γ was tested for its capacity to lead to the synthesis of C₄-HSL. PAO1 was exposed to 100 ng/ml of IFN-γ and C₄-HSL was measured in bacterial supernatants. C₄-HSL synthesis was increased in PAO1 exposed to IFN-γ (FIG. 13C). To verify that activation of the QS system by IFN-γ led to the production of other QS-dependent virulence products, production of pyocyanin, a redox active compound, was measured in PAO1 at various phases of growth following exposure to IFN-γ. Results showed that IFN-γ increased pyocyanin production in PAO1 (FIG. 13D). Finally, to determine whether rhlI and rhlR are required for the production of pyocyanin (PCN) and PA-I expression in response to IFN-γ, an rhlT mutant P. aeruginosa strain and, independently, an rhlR⁻mutant P. aeruginosa strain, were exposed to IFN-γ. PCN production and PA-I expression induced by IFN-γ were abolished in these mutant strains (FIGS. 13E, 13F). These data indicate that the QS system plays a key role in the response of P. aeruginosa to IFN-γ.

### Example 9

### Interferon-γ Binds to the Surface of P. aeruginosa

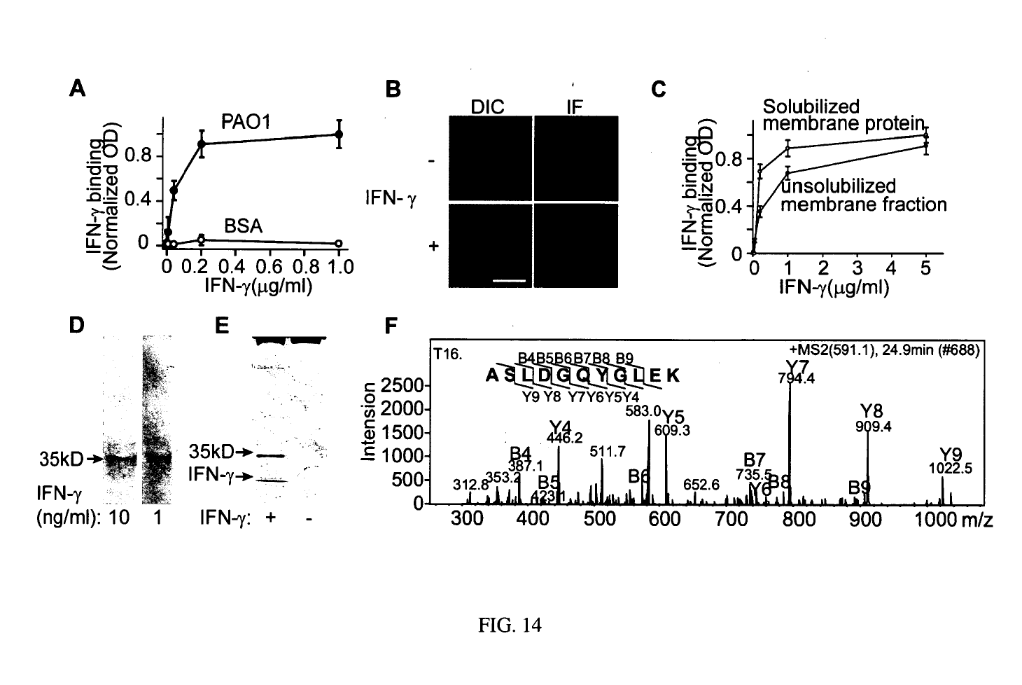

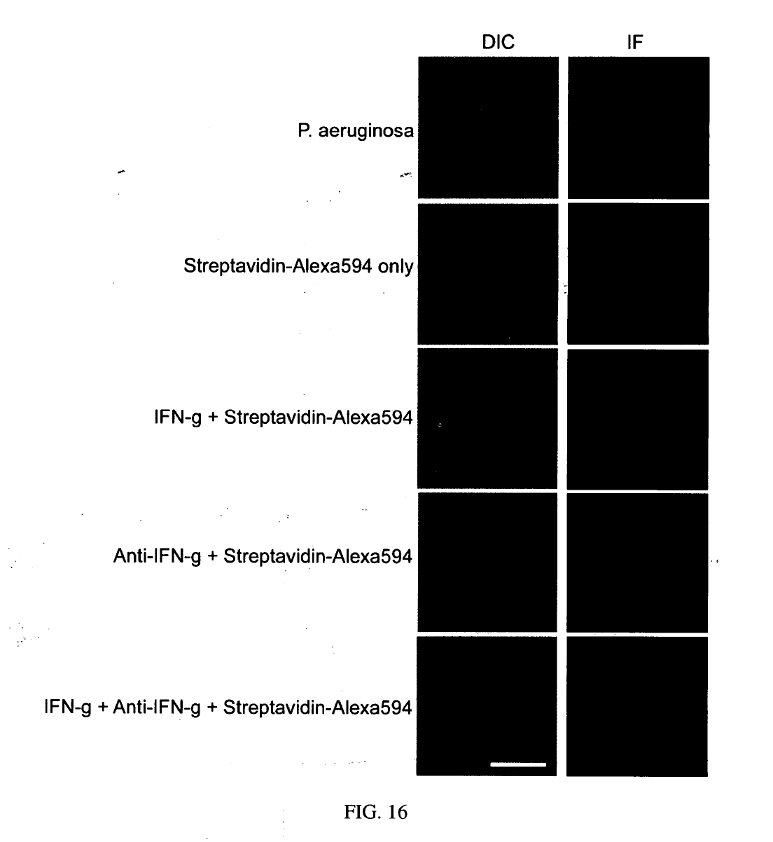

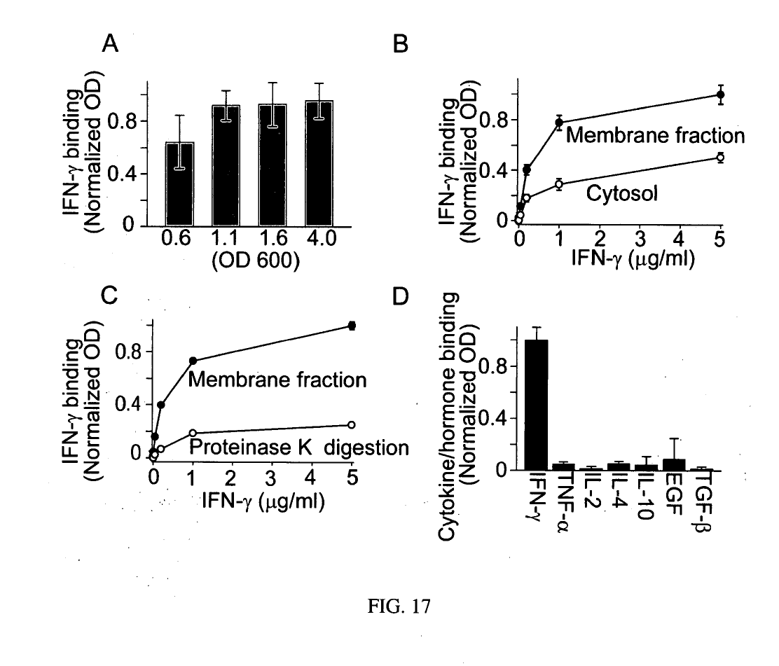

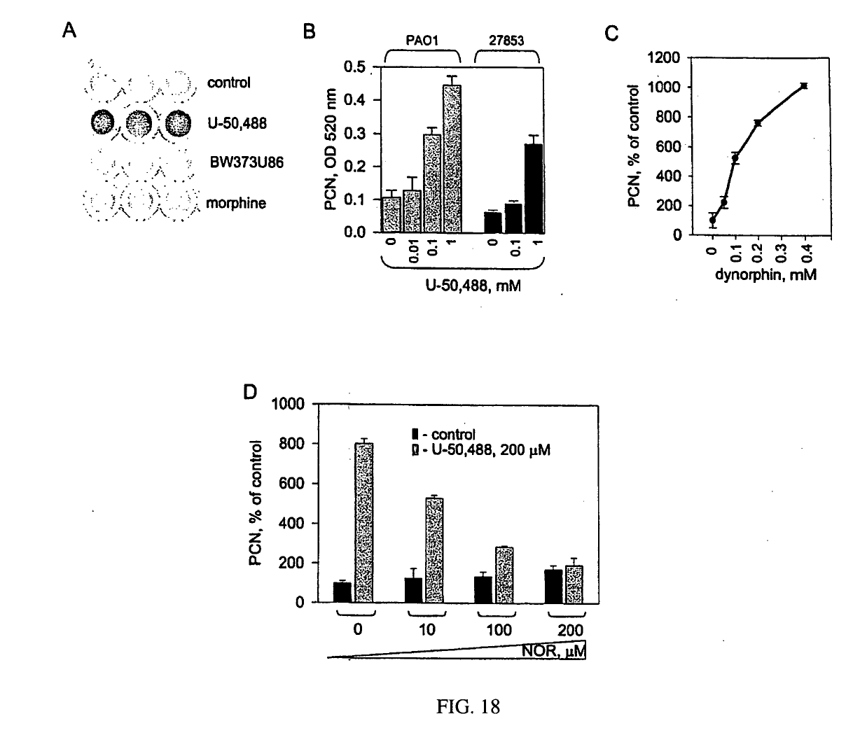

IFN-γ direct binding to a protein on the surface of P. aeruginosa, in the course of virulence activation, was also investigated. ELISA binding assays were performed by first coating microtiter plates with P. aeruginosa (strain PAO1), then adding recombinant human IFN-γ (rH IFN-γ), followed by biotin-labeled anti-IFN-γ antibody. IFN-γ avidly bound to whole fixed cells of P. aeruginosa in a dose-dependent manner (FIG. 14A). The ELISA data were confirmed by the results of immuno fluorescent imaging of bacterial cells exposed to IFN-γ followed by biotin-labeled anti-IFN-γ antibody and Alexa 594-labeled streptavidin. The vast majority of bacterial cells (73%±3.2%> vs. 8.5%±2.5%) bound IFN-γ (FIG. 14B, FIG. 16). The binding capacity of IFN-γ to P. aeruginosa was affected by bacterial growth phase (FIG. 17A). In order to localize the binding site of IFN-γ to P. aeruginosa (PAO1), equal protein concentrations of membrane and cytosol fractions of P. aeruginosa were prepared and coated onto ELISA microtiter plates. ELISA binding assays showed that IFN-γ preferentially bound to membrane fractions of P. aeruginosa (FIG. 17B). To determine if the observed membrane binding by IFN-γ was protein dependent, membrane fractions were treated with proteinase K for 3 hours and IFN-γ binding assessed. Binding by IFN-γ to P. aeruginosa membranes after treatment with proteinase K was decreased (FIG. 17C) suggested that IFN-γ binds to protein on the bacterial cell membrane. Next, other cytokines were examined to determine if they similarly would bind to P. aeruginosa cell membranes by performing reiterative binding studies with human TNF-α, IL-2, IL-4, IL-10, EGF, and TGF-β No binding was observed with any of these cytokines (FIG. 17D). Taken together, these data indicate IFN-γ bound to membrane protein on P. aeruginosa.

To isolate the putative protein to which IFN-γ binds on the cell membrane of P. aeruginosa, membrane proteins solubilized with mild detergents were initially shown to retain their binding capacity to IFN-γ by ELISA (FIG. 14C). Prior to isolation of the putative binding protein of IFN-γ, the binding of IFN-γ to single or multiple P. aeruginosa membrane proteins was investigated. Membrane proteins were separated by non-denaturing gel electrophoresis, transferred to PVDF membranes and hybridized with IFN-γ followed by biotin-labeled anti-IFN-γ antibody. Results demonstrated a single immunoreactive band of about 35 kD. Immunoreactivity was IFN-γ dose-dependent (FIG. 14D). In order to identify the putative binding protein, membrane protein was extracted from 4 L of freshly grown P. aeruginosa and fractionated by molecular weight between 10-100 kD. Solubilized protein was then immunoprecipitated using IFN-γ and anti-IFN-γ antibody. BSA was used as a control. Immunoprecipitation resulted in the appearance of a distinct protein with a molecular weight of about 35 kD. To further confirm that the protein isolated by immunoprecipitation was dependent on the presence of IFN-γ, equally divided solubilized membrane protein fractions were mixed with and without IFN-γ and then immunoprecipitated with anti-IFN-γ antibody. The 35 kD band appeared only in the solubilized membrane protein mixed with IFN-γ (FIG. 14E). The IFN-γ-dependent band was identified by ESI-TRAP-Electrospray LC-MSMS Ion Trap as the P. aeruginosa outer membrane porin OprF (FIG. 14F). These data established that IFN-γ binds to the P. aeruginosa outer membrane protein OprF.

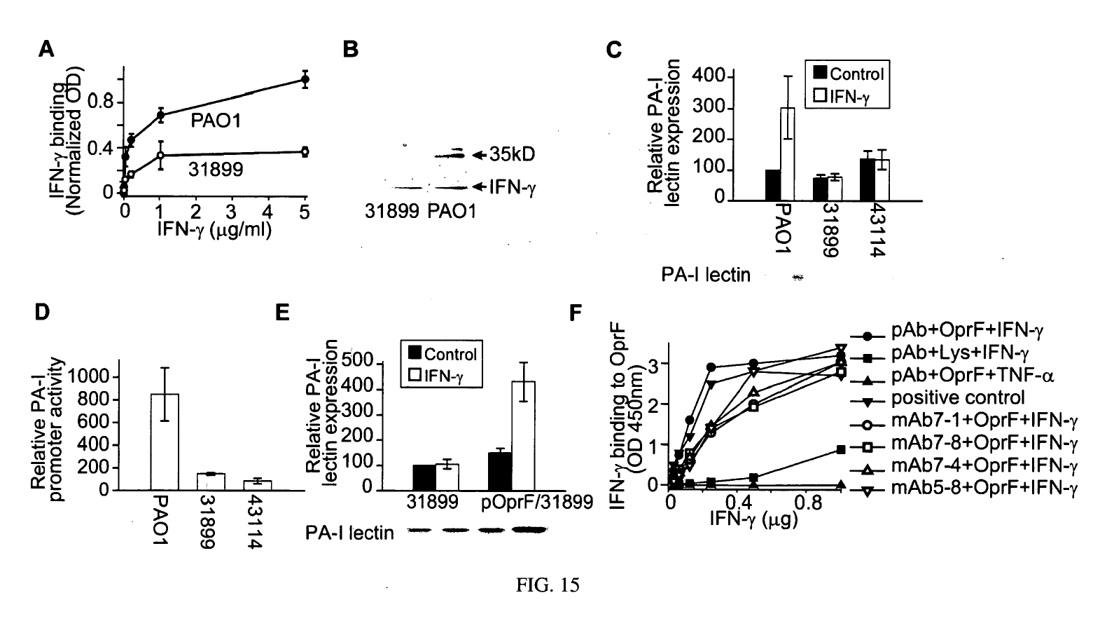

To verify that OprF represented the major binding site for IFN-γ in P. aeruginosa strain PAO1, solubilized membrane proteins from OprF knockout strains of P. aeruginosa strain PAO1 (Jacobs et al., Proc. Natl. Acad. Sci. (USA) 100:14339, 2003, were tested for their ability to bind IFN-γ in comparison to the wild-type strain using the established ELISA and immunoprecipitation technique. ELISA binding assays of solubilized membrane proteins demonstrated reduced binding of IFN-γ in OprF⁻ strains (FIG. 15A). Immunoprecipitation of solubilized membrane protein using IFN-γ and specific antibody confirmed the role of OprF by showing complete loss of the approximately 35 kD band in the OprF mutant strain (FIG. 15B). To verify the functional role of OprF in the responsiveness of P. aeruginosa to IFN-γ, the expression of the PA-I protein in wild-type and OprF mutant strains exposed to 100 ng/ml of IFN-γ was examined. Results demonstrated that mutant strains failed to increase the expression of the PA-I protein in response to an effective stimulating dose of IFN-γ as compared to the wild-type strain (FIG. 15C). The results from reporter gene fusion of wild-type and OprF mutant strains also demonstrated that IFN-γ activated PA-I expression through OprF (FIG. 15D). To further verify the role of OprF, OprF was reconstituted in mutant P. aeruginosa strain 31899 using the plasmid pUCP24/OprF, which expresses OprF. Reconstituted strains demonstrated recovery of their responsiveness to IFN-γ with an increase in PA-I protein expression (FIG. 15E). Finally, the binding between OprF and IFN-γ was verified by showing that purified OprF directly binds human IFN-γ (FIG. 15F) in a dose-dependent manner.

### Example 10

### Identification of Host Stress-Derived BSCs by Screening Candidate Agents: The Role of Endogenous Opioids

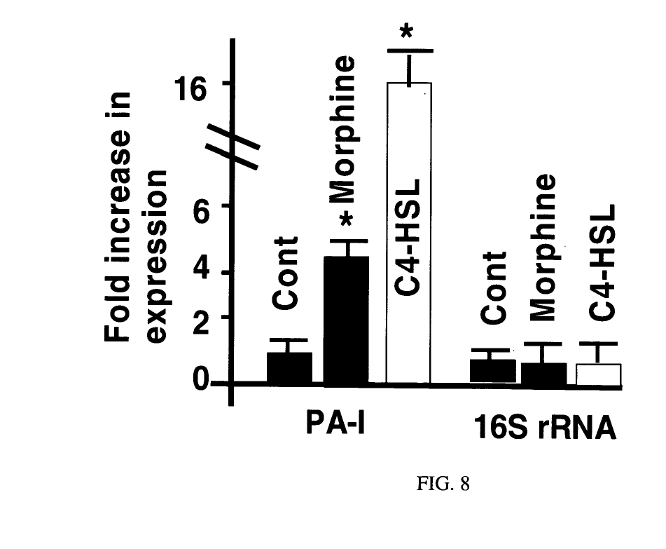

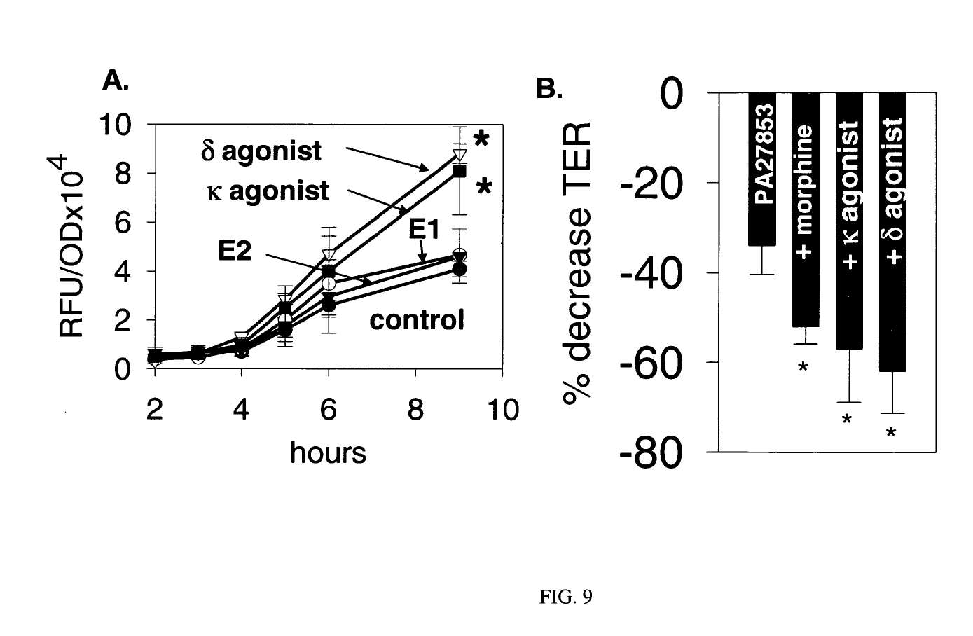

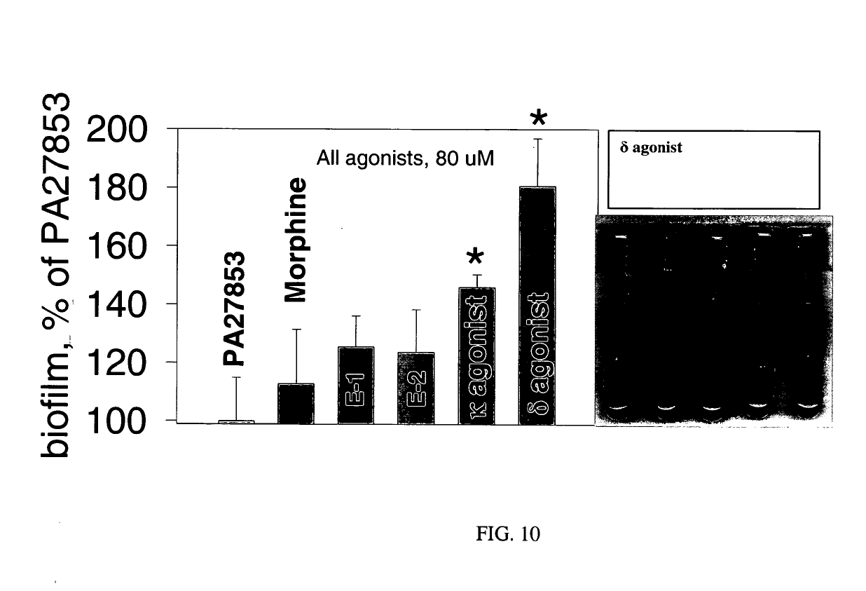

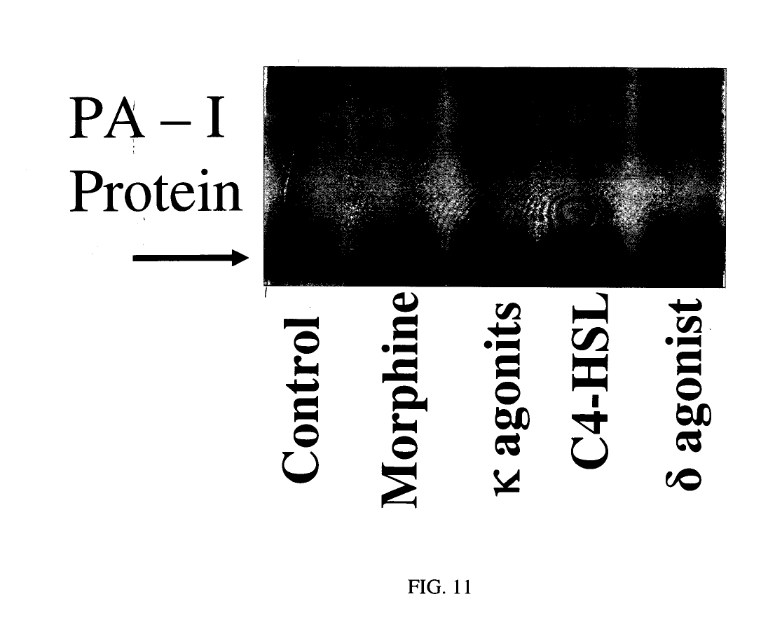

Although it was known that the counter-regulatory hormone, norepinephrine, increased the binding of P. aeruginosa to human O erythrocytes, there has been no information relating to the involvement of PA-I in the process. Accordingly, an assay to detect the presence of extracellular PA-I was performed. It was possible that norepinephrine would function as a host BSC for P. aeruginosa and, thus, affect human O erthyrocytes in a manner similar to the way it affected E. coli. Despite extensive analyses, PA-I expression was not affected by this compound. The screening of other catecholamines, all without positive results, led to the expectation that opioids, particularly morphine alkaloids, would activate PA-I. Endogenous morphine has been documented to be released in direct proportion to the magnitude of surgical stress/injury in both animals and humans. Initially, morphine was assessed for its effects. Interestingly, exposure of Pseudomonas strain PA27853 to physiologic concentrations of morphine (13 μM) resulted in a four-fold increase in PA-I expression (FIG. 8). As morphine is considered to be a non-selective opioid, specific endogenous opioid agonists with high selective affinity to μ, κ and δ receptors were tested for their abilities to induce PA-I lectin/adhesin expression in strains PA27853 and PAO1. Also tested were two pure u peptide agonists, endomorphine-1 (E1) (Tyr-Pro-Trp-Phe-NH₂) and endomorphine-2 (E2) (Tyr-Tyr-Pro-Phe-Phe-NH₂), the potent κ opioid non-peptide agonist U-50488, and the potent δ opioid non-peptide agonist BW373U86 for their respective abilities to induce PA-I expression in the reporter strain P. aeruginosa PA27853/PLL-EGFP (FIG. 9A). Results demonstrated that agonists targeting the κ and δ receptors had the greatest effect on PA-I expression as judged by increased fluorescence of the GFP reporter strain. In order to determine if PA-I was functionally expressed when exposed to the various opioid agonists, the agonists were tested for their abilities to shift the barrier dysregulating dynamics of PA27853 in MDCK cells. Results showed that all three of the opioids that induced PA-I expression (morphine, κ and δ agonists), shifted the virulence of PA27853, as judged by a more profound decrease in the TEER of MDCK cells following apical exposure (FIG. 9B).

In order to determine if morphine could shift the in vivo virulence of P. aeruginosa, mice were implanted with slow release morphine pellets that release a daily dose of morphine that is similar to that used clinically (pellets obtained from the National Institute on Drug Abuse (NIDA). Control mice were implanted with a placebo pellet. Mice drank infant formula spiked with a daily inoculum of 1×10⁸ cfu/ml of PA27853. All the morphine treated mice developed severe sepsis (4/4) and significant mortality while none of the control mice appeared septic and all survived. Finally, agonists were tested for their ability to induce biofilm in PA27853, a quorum sensing-dependent phenotype. Biofilm production by P. aeruginosa and other organisms has been established as a major phenotypic indicator of enhanced virulence. The κ and δ opioid agonists significantly increased biofilm production in strains PA27853 (FIG. 10). Taken together, these studies demonstrated that opioid agonists can directly influence the virulence, and potential lethality, of P. aeruginosa. It is expected that opioid agonists, whether found endogenously in a given cell type or not, and whether purified from a natural source, chemically synthesized, or produced by a combination thereof, are contemplated by the invention as useful modulators of the bacterial signaling affecting microbial pathogenesis generally, and eukaryotic (e.g., epithelial, endothelial) cell barrier function more specifically.

### Example 11

### Role of κ-Opioids in P. aeruginosa Virulence Expression

Opioid compounds, known to accumulate in tissues such as the lung and intestine following stress, directly activate the virulence of P. aeruginosa, as judged by pyocyanin production, biofilm formation, and the expression of the PA-IL protein. Specifically, pyocyanin production was enhanced in the presence of the selective κ-opioid receptor agonist. U-50,488, and the naturally occurring endogenous peptide dynorphin, also a selective κ-opioid receptor agonist. To understand the regulatory pathway(s) involved in opioid-induced virulence gene expression in P. aeruginosa, the effect of U-50,488 on multiple mutant P. aeruginosa strains defective in key elements involved in pyocyanin production was examined. Results demonstrated that the global transcriptional regulator. MvfR, plays a key role in pyocyanin production in response to U-50,488. Intact MvfR was also shown to be required for P. aeruginosa to respond to C4-HSL, a key quorum sensing signaling molecule known to activate hundreds of virulence genes. Taken together, these studies indicate that opioid compounds serve as host-derived signaling molecules that can be perceived by bacteria during host stress for the purposes of activating their virulence phenotype.

The experiments described herein used P. aeruginosa strains PAO1 and 27853, and their derivative strains (Table 1), which were routinely grown in tryptic soy broth (TSB) supplemented when necessary with tetracycline (Tc), 60 μg/ml, and/or gentamicin (Gm), 100 μg/ml. Alkaloid opiates morphine, a preferable μ-opioid receptor agonist, U-50,488, a specific κ-opioid receptor agonist, and BW373U86, a specific δ-opioid receptor agonist, along with the peptide opioid dynorphin, a specific κ-opioid receptor agonist, and specific κ-opioid-receptor antagonist nor-binaltorphimine were used in the experiments. Morphine was purchased from Abbott Laboratories, U-50,488, BW373U86, dynorphin, nor-binaltorphimine, and methyl anthranilate from Sigma-Aldrich, and C4-HSL from Fluka.

Amplified mvfR was directly cloned in pCR2.1 (Invitrogen), digested with XbaI-HindIII restriction endonucleases and subcloned into pUCP24 under the P_{lac} promoter to create pUCP24/mvfR. The plasmids pUCP24 (blank control) and pUCP24/mvfR were electroporated into strain 13375, defective in MvfR production, to create the P. aeruginosa strain 13375/MvfR (Tables 1, 2).

The gacA gene, a member of a two-component signaling method involved in the elaboration of virulence in many gram-negative bacteria, was amplified and directly cloned into pCR2.1 (Invitrogen). The gene was then excised with XbaI-HindIII restriction endonucleases and subcloned into pUCP24 under the P_{lac} promoter to create pUCP24/gacA. The plasmids pUCP24 (blank control) and pUCP24/gacA were electroporated into P. aeruginosa strain PAO6281, defective in GacA production, to create the P. aeruginosa strain PAO6281/GacA (Tables 1,2).

Truncation of MvfR was achieved by generating PCR products of truncated mvfR genes amplified from pUCP24/MvfR and their respective primers (Tables 1, 2). The PCR products were purified using a Geneclean kit (Qbiogene), digested with XbaI-HindIII restriction endonucleases, and ligated into pUCP24 followed by electroporation into P. aeruginosa strain 13375.

For pyocyanin assays, bacteria were grown in TSB at 37° C. under shaking conditions at 220 rpm, with opioid compounds added at the early exponential phase of bacterial growth (OD_{600 nm} of about 0.15-0.2). After incubation, pyocyanin was extracted from culture media in 6 chloroform extracts followed by re-extraction in 0.2 M HCl, and measured at OD_{520 nm}, as described (Essar et al., Bacteriol, 172:884, 1990).

Immunoblotting and fluorescence of the GFP-PA-IL reporter strain were used in PA-IL assays to determine the effect of opioids on PA-IL expression. For immunoblotting, P. aeruginosa PAO1 was grown in TSB media with or without 100 μM U-50,488, and cells were collected at the late exponential phase of growth (OD_{600 nm}=1.8). Equal amounts of protein from each sample were separated by 15% SDS-PAGE, transferred to a PDF membrane, and probed with affinity-purified rabbit polyclonal anti-PA-IL antibodies. The probed membranes were treated with anti-rabbit horseradish peroxidase-conjugated IgG, and developed using SuperSignal West Femto chemiluminescent substrate (Pierce). For PA-IL expression detected by fluorescence, a bacterial culture of the GFP-PA-IL reporter strain 27853/PLL-EGFP (Wu, et al., Gastroenterology, 126, 488, 2004, incorporated herein by reference) was plated at a final concentration of 10⁸ CFU/ml in a 96-well fluorometry plate (Costar) in HDMEM media containing 10% FBS and HEPES buffer with or without 60 μM of U-50,488. Incubation was performed at 37° C., 100 rpm, and fluorescence reading was performed hourly with a 96-well fluorometry Plate Reader (Synergy HT, Biotec Inc.) at excitation/emission of 485/528 nm. Fluorescence intensity was normalized to cell density measured at 600 nm.

Biofilm formation assays were conducted by initially plating bacterial cells in quadruplicate in 96-well U-bottom plates (Falcon) at a concentration of 10⁷ CFU/ml in M63S media (13.6 g KH₂PO₄ 1-1, 2.0 g (NH₄)₂SO₄ 1-1, 0.5 mg FeSO₄x7H₂O 1-1), supplemented with 0.5% casamino acids, 1 mM MgSO₄x7H₂0 and 0.2% glucose, and incubating overnight at 37° C. under static conditions. U-50,488 was added at the inoculation point. After inoculation, the wells were rinsed thoroughly with water and the attached material was stained with 0.1% crystal violet, washed with water, and solubilized in ethanol. Solubilized fractions were collected and absorbance measured at 590 nm as described (O'Toole et al., Mol Microbiol, 28, 449, 1998, incorporated herein by reference) with a Plate Reader.

The experiments disclosed herein establish that the κ-opioid receptor agonists U-50,488 and dynorphin stimulate pyocyanin production in P. aeruginosa. Pyocyanin, a redox active compound that increases intracellular oxidant stress, has been shown to play a key role in the virulence of P. aeruginosa in animal models mediating tissue damage and necrosis during lung infection (Lau, et al., Infect Immun, 72, 4275, 2004). P. aeruginosa PAO1 was exposed to peptide opioids and alkaloid opiates representing groups of μ-, κ-, and δ-opioid receptor agonists. Results indicated that following overnight exposure, the alkaloid opiate U-50,488, a specific κ-opioid receptor agonist, induced an intensely bright green color in P. aeruginosa PAO1, while no such effect was observed with any of the remaining compounds (FIG. 18A). To verify that the color change was due to PCN production, pyocyanin was measured at OD_{520 nm}. Results demonstrated that U-50,488 induced a dose-dependent effect on PCN production that was observed with P. aeruginosa strains PAO1 and 27853 (FIG. 18B). Exposure of P. aeruginosa to dynorphin, a naturally occurring specific κ-opioid receptor peptide agonist, also enhanced PCN production in a dose-dependent manner (FIG. 18C). Reiterative experiments performed in the presence of the specific κ-opioid receptor antagonist norbinaltorphimine (NOR), demonstrated that NOR attenuated enhanced PCN production in PAO1 following exposure to U-50,488 in a dose-dependent manner and completely inhibited enhanced PCN production at a dose of 200 μM (FIG. 18D).

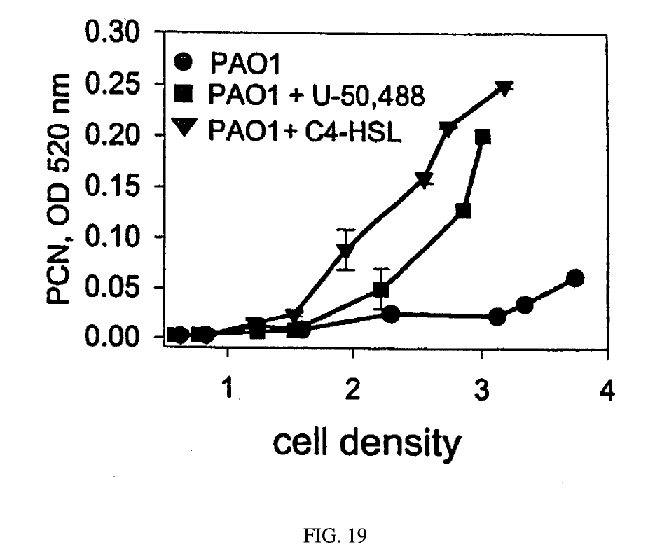

The dynamics of PCN production in response to U-50,488 at varying cells densities was assessed, since the expression of QS-dependent genes is known to occur at high bacterial cell densities when QS signaling molecules reach their threshold concentrations. Results showed that the κ-opioid-receptor agonist U-50,488 shifted pyocyanin production to lower cell densities in P. aeruginosa. As a positive control, bacteria were exposed to C4-homoserine lactone (C4-HSL), a QS signaling molecule involved in PCN regulation. Exposure of PAO1 to U-50,488 had a similar effect to exposure of cells to C4-HSL, resulting in a shift in the production of PCN to lower cell densities (FIG. 19). Neither compound had an effect on bacterial growth in TSB media.

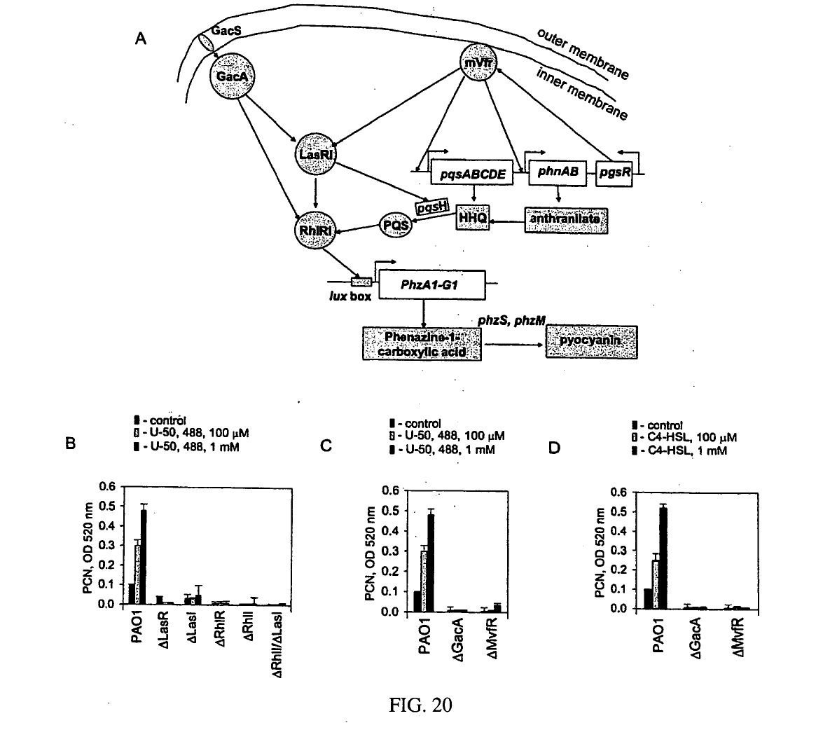

Additional experiments demonstrated that the κ-opioid-receptor agonist U-50,488 exerts its inducing effect on pyocyanin production via elements of the quorum sensing system in Pseudomonas aeruginosa. A summary of the pathways of PCN regulation and biosynthesis is depicted in FIG. 20A. In order to define potential pathways by which U-50,488 induces PCN production, mutant strains defective in key genes involved in PCN production were exposed to U-50,488 and the effect on pyocyanin production was measured. First, mutants defective in genes encoding core elements of the QS system (lasR, lasI, rhlI, rhlR) were analyzed and the results demonstrated that exposure to U-50,488 did not restore PCN production (relative to non-mutant strains) in any of these mutants (FIG. 20B). The roles of the global virulence regulators GacA and MvfR on PCN production were then investigated. Both GacA and MvfR have been shown to play a major role in PCN production in P. aeruginosa. Neither ΔGacA nor ΔMvfR produced PCN, as expected, and exposure to U-50,488 could not restore PCN production (FIG. 20C). C4-HSL was also unable to restore PCN production in the gacA and mvfR mutants (FIG. 20D). The finding that C4-HSL did not restore PCN production in the GacA mutant is consistent with the finding that the analogous QS molecule, N-hexanoyl-HSL (C6-HSL), did not restore phenazine production in a ΔGacA mutant of P. aureofaciens. Seven additional mvfR mutants from the comprehensive transposon library (Jacobs, et al., Proc Natl Acad Sci (USA) 100, 14339, 2003) (i.e., numbers 8902, 47418, 35448, 51955, 21170, 47853, and 47198) were exposed to C4-HSL in order to confirm this finding. Results demonstrated that none of these mutants produced PCN in the presence of 1 mM C4-HSL.

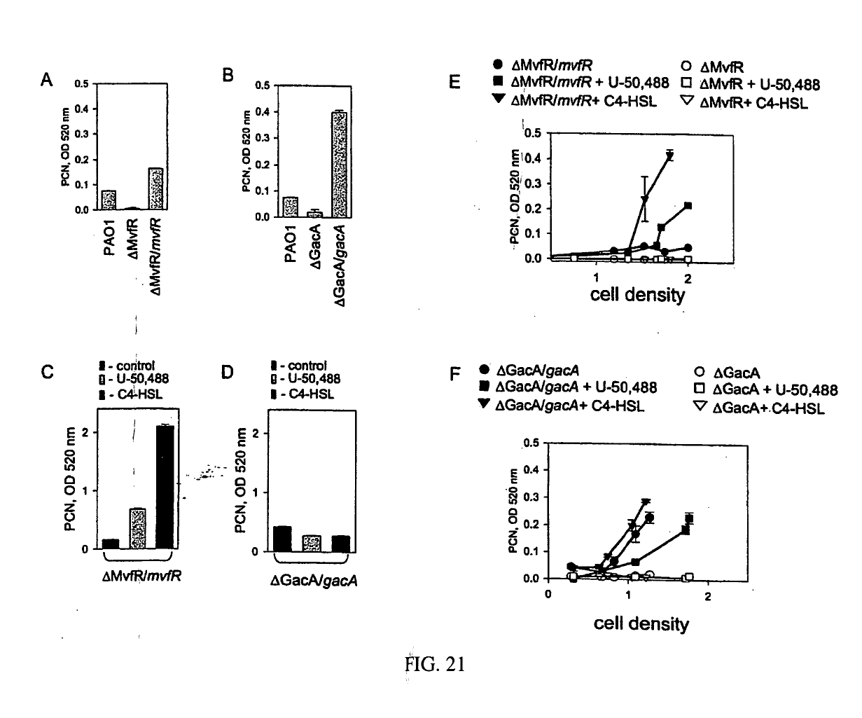

Further experiments showed that MvfR is involved in the ability of U-50,488 and C4-HSL to enhance PCN production in PAO1. In order to define the possible role of MvfR and GacA in the U-50,488-mediated upregulation of PCN synthesis, ΔMvfR and ΔGacA mutations were complemented with their respective wild-type genes on the multicopy plasmid pUCP24 (West, et al., Gene 148, 81, 1994). Both complemented mutants produced significantly higher amounts of PCN (FIGS. 21A,B). The addition of C4-HSL and U-50,488 further increased the already elevated PCN production in ΔMvfR /mvfR (FIG. 21C). In contrast, PCN production in ΔGacA/gacA was decreased, albeit minimally, when exposed overnight to either 1 mM U-50,488 or 100 μM C4-HSL (FIG. 21D). Dynamic tracking of PCN production in the complemented mutant ΔMvfR/mvfR exposed to U-50,488 and C4-HSL demonstrated a shift in PCN production at lower cell densities (FIG. 21E), similar to that observed in the parental strain PAO1 (FIG. 19). The gacA complemented mutant, ΔGacA/gacA, itself produced PCN at lower cell densities than those observed with the parental strain PAO1. Exposure of ΔGacA/gacA to C4-HSL had no effect on the dynamics of PCN production, whereas exposure to U-50.488 delayed PCN production. (FIG. 21F).

These results indicated that MvfR is involved in the up-regulation of PCN production by exogenously applied U-50,488 and C4-HSL.

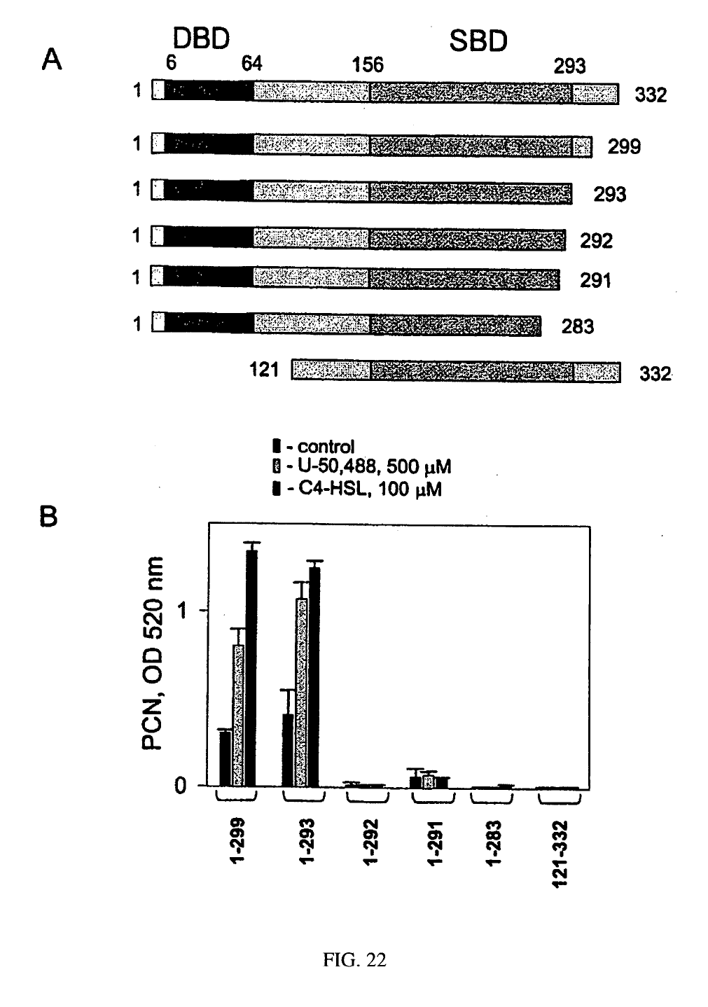

Intact substrate-binding and DNA-binding domains of MvfR have been shown to be required for U-50,488 enhancement of PCN production in PAO1. MvfR belongs to a family of prokaryotic LysR transcriptional regulators that possess a helix-turn-helix DNA-binding motif at the N-terminus and a substrate binding domain at the C-terminus. A NCBI Conserved Domain Search revealed similar domains in MvfR: a LysR DNA-binding domain located at amino acids 6-64, and a LysR substrate binding domain located at amino acids 156-293 (FIG. 22A). Therefore PAO1 mutants producing N- and C-terminus-truncated MvfR were constructed to determine if specific domains could be identified that play a functional role in mediating the κ-opioid receptor agonist effect on PCN production. Results indicated that the mutant lacking amino acids 121-332, defective in the DNA-binding domain, did not produce any PCN, and did not respond to U-50,488 or C4-HSL (FIG. 22B). Mutants lacking either amino acids 1-299 or 1-293, truncated at their C-termini without affecting the substrate binding domain, produced PCN and responded to U-50,488 and C4-HSL with enhanced PCN production. Further deletions, however, including amino acids Arg293, Leu292, and Phe284, did affect the substrate binding domain in mutants 1-292, 1-291, and 1-283. All three mutants failed to produce PCN and did not respond to U-50,488 and C4-HSL (FIG. 22B). These results confirm a key functional role for MvfR in mediating enhanced PCN production in P. aeruginosa in response to U-50,488 and C4-HSL.

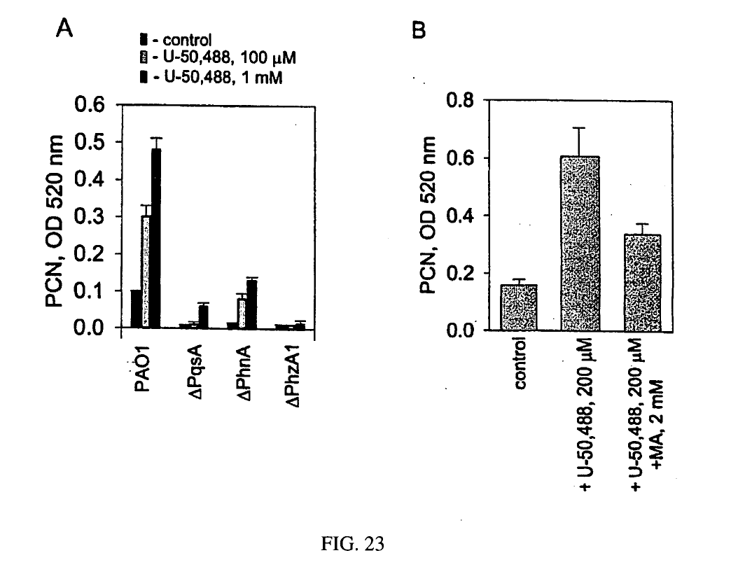

The data further revealed that the effect of U-50,488 on PCN production is dependent on MvfR-regulated synthesis of Pseudomonas Quinolone Signal (PQS). MvfR might play a critical role in PCN production via positive transcriptional regulation of the phnAB and PQS ABCDE operons that encode two 12 precursors of PQS, anthranilic acid (AA) and 4-hydroxy-2-heptylquinolone (HHQ) (Deziel, et al., Proc Natl Acad Sci (USA) 101, 1339, 2004, incorporated herein by reference). Therefore, the mutants APhnA and ΔPqsA were examined for their ability to produce PCN in the presence of U-50,488. Neither mutant produced PCN. Exposure of each mutant to U-50,488 resulted in a slight increase in PCN production, although the increase was much less than that observed with the wild-type strain PAO1 (FIG. 23A). These data indicated that MvfR-regulated PQS synthesis is important for the ability of U-50,488 to enhance PCN production. Finally, reiterative experiments were performed with a P. aeruginosa mutant defective in the phzA1 gene, which is part of the operon that contains the core genes for PCN biosynthesis and that is directly preceded by the lux box. ΔPhA1 produced no PCN even when exposed to U-50,488 (FIG. 23A).

To confirm that PQS plays a role in the pathway by which U-50,488 enhances PCN production, U-50,488 was applied to P. aeruginosa incubated with 2 mM methyl anthranilate (MA), a compound previously shown to inhibit PQS synthesis in P. aeruginosa.) Results demonstrated that MA inhibited the ability of U-50,488 to enhance PCN production in PAO1 (FIG. 23B). These findings indicated that U-50,488 triggers PCN production in P. aeruginosa via a signal transduction pathway that includes the activation of transcriptional regulator MvfR and the synthesis of the MvfR-regulated molecule, PQS.

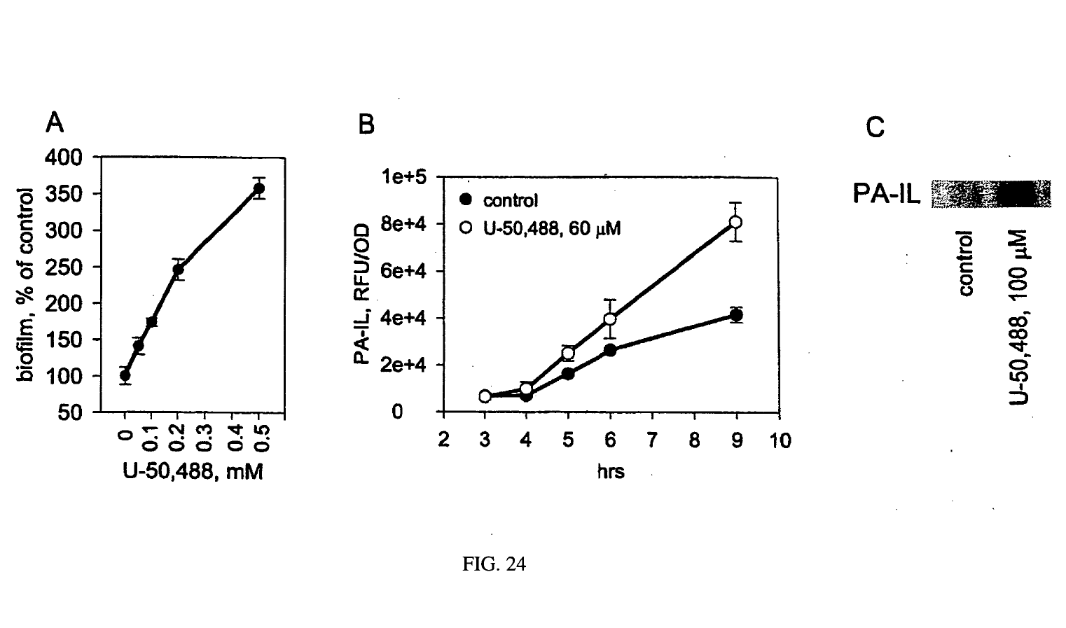

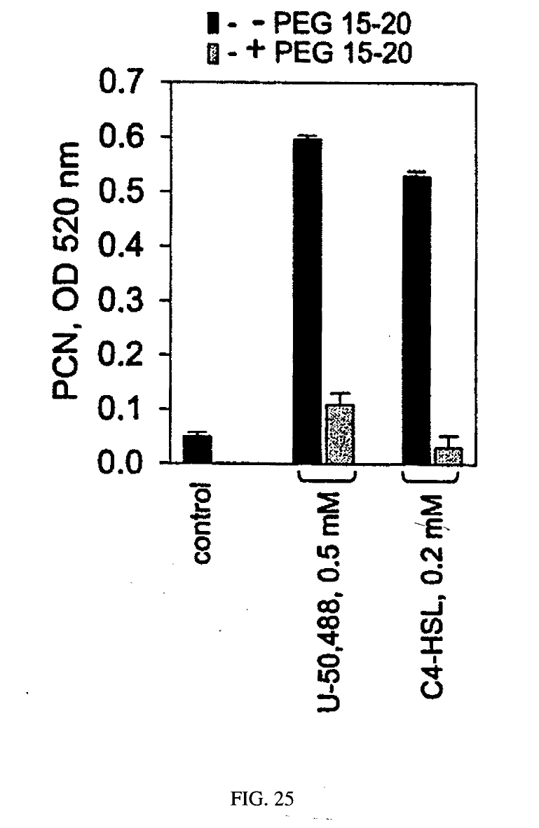

U-50,488 also stimulated other QS-regulated virulence determinants in P. aeruginosa, including biofilm formation and PA-IL production. To determine if other QS-dependent phenotypes could be expressed in response to U-50,488, biofilm production was measured and PA-IL lectin expression in P. aeruginosa exposed to this opiate. U-50,488 enhanced biofilm formation in PAO1 in a concentration-dependent manner (FIG. 24A). PA-IL expression was dynamically tracked in response to U-50,488 using the green fluorescent PA-IL reporter strain P. aeruginosa 27853/PLL-EGFP (Wu, et al., 2004, supra). Marked fluorescence was observed in this strain following 9 hours of growth in HDMEM media (FIG. 24B). Results were confirmed in strain PAO1 by immunoblotting using rabbit polyclonal antibody against PA-IL (FIG. 24C).

Experimental results also established that the effect of U-50,488 on PCN production in P. aeruginosa can be inhibited by the anti-infective high molecular weight polymer HMW PEG 15-20. A high molecular weight polymer, PEG 15-20, protects mice against lethal sepsis due to P. aeruginosa by interfering with the ability of both host elements (epithelial cell contact) and the QS signaling molecule C4-HSL to enhance P. aeruginosa virulence without affecting bacterial growth. The capacity of HMW PEG 15-20 to interfere with the U-50, 488 effect on P. aeruginosa was assessed by measuring PCN production in the media of P. aeruginosa PAO1 incubated in the presence of 5% HMW PEG 15-20 and 0.5 mM U-50,488 or 0.2 mM C4-HSL. Results demonstrated that HMW PEG 15-20 had a strong inhibitory effect on both U-50, 488- and C4-HSL-mediated up-regulation of PCN production.

TABLE 1
Bacterial strains

| P. aeruginosa strains | Relevant genotype | Source |
|---|---|---|
| PA27853 | Wild type | Dr. Alverdy |
| PAO1 | Wild type | Dr. Iglewski |
| PAO-JP-1 | ΔLasl (lasl::Tc^{r}) | Dr. Iglewski (21) |
| PAO-R1 | ΔLasR (lasR::Tc^{r}) | Dr. Iglewski |
| PDO100 | ΔRhlI (rhlI::Tn501) | Dr. Iglewski |
| PAO-MW1 | ΔRhlIΔLasI (rhlI::Tn501 lasI::tetA) | Dr. Greenberg (30) |
| PAO44488 | ΔRhlR (rhlR::ISphoA/hah) | PAO1 transposon library (24) |
| PAO6281 | ΔGacA (gacA::Sp^{r}/Sm^{r}) | Dr. Reimmann (22) |
| PAO6281/pUCP24/GacA | ΔGacA complemented with gacA | This study |
| | on pUCP24 | |
| PAO6281/pUCP24 | ΔGacA transformed with blank | This study |
| | pUCP24 | |
| PAO8902 | ΔMvfR (mvfR:: ISlacZ/hah) | PAO1 transposon library |
| PAO47418 | ΔMvfR (mvfR:: ISphoA/hah) | PAO1 transposon library |
| PAO35448 | ΔMvfR (mvfR:: ISphoA/hah) | PAO1 transposon library |
| PAO51955 | ΔMvfR (mvfR:: ISphoA/hah) | PAO1 transposon library |
| PAO21170 | ΔMvfR (mvfR:: ISlacZ/hah) | PAO1 transposon library |
| PAO47853 | ΔMvfR (mvfR:: ISphoA/hah) | PAO1 transposon library |
| PAO47198 | ΔMvfR (mvfR:: ISphoA/hah) | PAO1 transposon library |
| PAO13375 | ΔMvfR (mvfR:: ISlacZ/hah) | PAO1 transposon library |
| PAO13375/pUCP24/MvfR | ΔMvfR complemented with mvfR | This study |
| | on pUCP24 | |
| PAO13375/pUCP24 | ΔMvfR transformed with blank | This study |
| | pUCP24 | |
| PAO53589 | ΔPqsA (pqsA:: ISphoA/hah) | PAO1 transposon library |
| PAO37309 | ΔPhzA (phzA:: ISphoA/hah) | PAO1 transposon library |
| PAO47305 | ΔPhzA1 (phzA1:: ISphoA/hah) | PAO1 transposon library |
| PAO3375/pUCP24/MvfR | ΔMvfR complemented with pUCP24 | This study |
| 1-299 | harboring mvfR truncated with 33 | |
| | amino acids at the C terminus | |
| PAO13375/pUCP24/MvfR | ΔMvfR complemented with pUCP24 | This study |
| 1-293 | harboring mvfR truncated with 39 | |
| | amino acids at the C terminus | |
| PAO13375/pUCP24/MvfR | ΔMvfR complemented with pUCP24 | This study |
| 1-292 | harboring mvfR truncated with 40 | |
| | amino acids at the C terminus | |
| PAO13375/pUCP24/MvfR | ΔMvfR complemented with pUCP24 | This study |
| 1-291 | harboring mvfR truncated with 41 | |
| | amino acids at the C terminus | |
| PAO13375/pUCP24/MvfR | ΔMvfR complemented with pUCP24 | This study |
| 1-283 | harboring mvfR truncated with 49 | |
| | amino acids at the C terminus | |
| PAO13375/pUCP24/MvfR | ΔMvfR complemented with pUCP24 | This study |
| 121-332 | harboring mvfR truncated with 120 | |
| | amino acids at the N terminus | |
| 27853/PLL-EGFP | Green fluorescent PA-IL reporter | Alverdy (13) |
| | strain | |

TABLE 2
Primers designed for complementation and truncation

| | | |
|---|---|---|
| Strain | Template | Primers |

| | | |
|---|---|---|
| 13375/MvtR | PAO1 DNA | forward 5′-AAGGAATAAGGGATGCCTATTCA-3′ |
| | | reversed 5′-CTACTCTGGTGCGGCGCGCTGGC-3′ |
| PAO281/GacA | PAO1 DNA | forward 5′-CGACGAGGTGCAGCGTGATTAAGGT-3′ |
| | | reversed 5′-CTAGCTGGCGGCATCGACCATGC-3′ |
| 13375/1-299 | pUCP24/mvfR | MvfrXbaI 5′-GCTCTAGAAAGGAATAAGGGATGCCTAT-3′ |
| | | C33HindIII 5′-CCCAAGCTTCTAACGCTGGCGGCCGAGTTC 3′ |
| 13375/1-293 | pUCP24/mvfR | MvfrXbaI 5′-GCTCTAGAAAGGAATAAGGGATGCCTAT-3′ |
| | | C39HindIII 5′-CCCAAGCTTCTAGCGCAGGCGCTGGCGGGC-3′ |
| 13375/1-292 | pUCP24/mvfR | MvfrXbaI 5′-GCTCTAGAAAGGAATAAGGGATGCCTAT-3′ |
| | | C40HindIII 5′-CCCAAGCTTCTATACAGGCGCTGGCGGGCGCT-3′ |
| 13375/1-291 | pUCP24/mvfR | MvfrXbaI 5′-GCTCTAGAAAGGAATAAGGGATGCCTAT-3′ |
| | | C41HindIII 5′-CCCAAGCTTCTAGCGCTGGCGGGCGCTTTC-3′ |
| 13375/121-232 | pUCP24/mvfR | N120XbaI |
| | | 5′-GCTCTAGAAAGGAATAAGGGATGGTCAGCCTGATACGC-3′ |
| | | MvfRHindIII |
| | | 5′-CCCAAGCTTCTACTCTGGTGCGGCGCGCTGGC-3′] |

### Example 12

### P. aeruginosa PAO1 Expresses Abundant PA-I and Alters MDCK Monolayer Permeability in a PA-I Dependent Manner

In order to verify that the sequenced P. aeruginosa strain, PAO1, expressed PA-I, and to verify that strains altered the TEER of MDCK cells in a PA-1-dependent manner, both wild-type and PA-I mutant strains deleted of the PA-I gene (lecA) (obtained from the comprehensive transposon mutant library, Jacobs et al., Proc Natl Acad Sci (USA) 100:14339-44, 2003), were assayed for PA-I protein expression and their abilities to decrease MDCK monolayer TEER. PA-I protein expression is highly abundant and responds to varying doses of C4-HSL, its cognate quorum sensing signaling molecule. In addition, in this strain, the ability of P. aeruginosa to decrease MDCK monolayer integrity (TEER) is highly dependent on the expression of PA-I. Also, it was determined that the PA-I induced permeability defect in MDCK cells was of sufficient magnitude to permit the apical to basolateral flux of exotoxin A across the monolayers. Finally PA-I protein was abundantly expressed in PAO1 when strains were exposed to the various opioid agonists. For PA-I protein, the δ agonist induced a response equal to C4-HSL. The data establish that PA-I expression affects eukaryotic cell barrier function. Thus, it is expected that modulators of PA-I expression, as well as modulators of PA-I activity, will be useful in affecting the virulence phenotype of microbial pathogens and will be useful in affecting the eukaryotic (e.g., epithelial, endothelial) cell barrier dysfunction associated with pathogenesis. Moreover, such modulators will be useful in treating a variety of mammals, and particularly humans, exhibiting disorders or diseases characterized by permeability defects, including epithelial and endothelial cell disorders exhibiting a cell barrier dysfunction; the modulators will also be useful in ameliorating at least one symptom of such a disorder and in preventing such a disorder.

### Example 13

### Host Cell-Derived Bacterial Signaling Components Enhance the Barrier Dysregulating Properties of P. aeruginosa Against Epithelial Cells

In order to demonstrate that host stress BSCs could shift the barrier dysregulating dynamics of P. aeruginosa against the epithelium, media and cell membrane fractions from Caco-2 cells exposed to hypoxia were added to the apical wells of MDCK cells apically inoculated with PA27853. TEER was measured over time. C4-HSL was also added to serve as a positive control for PA-I expression. Both media and cell membranes enhanced the barrier dysregulating properties of P. aeruginosa (PA27853) against MDCK cells at four hours. None of the host cell-derived bacterial signaling compounds alone had any effect on MDCK TEER. The results demonstrated that the microbial pathogen (e.g., P. aeruginosa) is necessary to alter the banner function of host cells, as demonstrated for epithelial cells and as reasonably expected for endothelial cells.

### Example 14

### PA-I is Expressed In Vivo within the Digestive Tube of Caenorhabditis elegans

The PA-I-GFP reporter plasmid was introduced by electroporation into P. aeruginosa strain PA14, a strain highly lethal to C. elegans. Worms were then fed GFP-tagged PA14 and PA27853 and examined for fluorescent bacteria. Worms feeding on lawns of PA14 and PA27853 displayed fluorescent bacteria within the digestive tube, whereas no fluorescence was seen within the surrounding media, indicating that PA-I promoter activity was activated by local factors within the worm digestive tube. Finally, the killing dynamics of strain PA-14, a highly lethal strain in this model, was compared to the dynamics associated with the completely sequenced PAO1 strain. The strain of E. coli (OP50) upon which worms normally feed, resulted in 100% survival, whereas, PA-14 displayed fast killing dynamics, as predicted. The PAO1 strain displayed slow killing with only a 50% mortality rate at 80 hours. Thus PAO1 exhibited killing dynamics that will allow assessments of whether host stress-derived BSCs shift the killing curve to that of a more virulent strain. It is expected that BSCs, whether soluble or membrane-bound, will shift the killing dynamics of relatively quiescent, or benign, microbes towards the dynamics exhibited by lethal microbial strains. Stated in the alternative, it is expected that a BSC will shift the phenotype of a microbe towards a virulent phenotype. Modulators of such activities are expected to be useful in preventing and treating disorders associated with the display of a virulence phenotype by any such microbe and, in particular, by P. aeruginosa. Such modulators are also expected to be used in methods for ameliorating a symptom of such a disorder.

### Example 15

### P. aeruginosa Genes Involved in BSC-Induced PA-I Lectin/Adhesin Gene Expression

The data demonstrate that i) morphine, the potent opioid agonists U-50488 and BW373U86, which target κ and δ receptors, respectively, and IFN-γ, induce a robust response in P. aeruginosa strains PA27853 and PAO1 to express PA-I; and that ii) PA-I expression is dependent on multiple elements of the virulence gene regulatory circuitry in P. aeruginosa, including the quorum sensing signaling system (QS) and RpoS. The data will show the genes that are required for opioids and IFN-γ to elicit a PA-I response in P. aeruginosa and will facilitate a determination of whether these host stress-derived BSCs use common genes and membrane receptor proteins to activate PA-I expression.

A. Genes Required for P. aeruginosa PA-I Expression Responsive to Morphine, κ and δ Opioid Agonists, and IFN-γ

At least two techniques are contemplated for use in gene identification: 1) transcriptome analysis on P. aeruginosa strain PAO1 exposed to morphine, κ and δ opioid receptor agonists, and IFN-γ, and 2) screening of transposon mutants of the candidate genes identified by transcriptome analysis for their ability to up-regulate PA-I protein expression in response to opioids and IFN-γ, thereby establishing a functional role for such genes.

### B. Transcriptome Analysis

Genes in strain PAO1 whose expression is increased in the presence of opioids and/or IFN-γ will constitute the initial focus. Transcriptome analyses are performed using Affymetrix GeneChip genome arrays in strain PAO1 to identify the genes that respond to the host cell elements such as morphine (non-selective opioid receptor agonist), U-50488 (κ receptor agonist). BW373U86 (δ opioid receptor agonist), and IFN-γ. Time and dose variables for the following experiments are based on data for PA-I expression (mRNA) in strain PAO1.

Briefly, bacteria are grown in TSB overnight and diluted 1:100 in TSB containing either morphine (20 μM), κ agonist (80 μM), δ agonist (80 μM), or IFN-γ (10 μg/ml). Bacteria are then grown to an OD₆₀₀ of 0.5, 1.0, and 2.0, representing three stages of growth: exponential phase, late exponential phase, and stationary phase, respectively. These three time points will permit the capture of genes that are expressed early in the PA-I signaling pathway as well as during time points of high cell density. For transcriptome analysis, RNA is isolated from bacterial cells (treated and non-treated with morphine, κ and δ opioid receptor agonists, and IFN-γ) at the three designated points in the growth phase. cDNA synthesis, fragmentation, labeling, and hybridization, as well as P. aeruginosa GeneChip genome array processing, are performed as described herein or as known in the art. Each experiment is preferably performed in triplicate.

### C. Functional Analysis of Candidate Genes

Genes showing at least a 2.5-fold change in expression resulting from exposure to morphine, κ and δ opioid receptor agonists, and/or IFN-γ, are individually tested for their specific role in PA-I protein expression by screening mutant strains from a PAO1 transposon library (University of Washington Genome Center, see below) using dot blot analysis. Briefly, strains are grown in sequential runs using 384-well microtiter plates at 2 separate bacterial cell densities (OD₆₀₀ of 1.0 and 2.0) predetermined to respond to the inducing compound (opioids, IFN-γ). Dose-response curves are generated with varying doses of the PA-I inducing compounds at different bacterial cell densities in wild-type strains and in several mutant strains to determine the optimal conditions for screening. Experiments are performed separately for morphine, U-50488, BW373U86, and IFN-γ. Briefly, either morphine, U-50488, BW373U86, or IFN-γ are added to the wells containing mutant strains at the predetermined dose. All runs are performed with the wild-type strain as a control. The PA-I-inducing compound is added to the well for a predetermined time. Next, the supernatant is removed and the bacterial cell pellet is lysed by the addition of lysis solution directly into the well. The entire 384-well plate is then spun down (4000 g) and the supernatant transferred to an Immobilon P-PDF membrane using a 384 replicator. Membranes are then treated with anti-PA-I primary and secondary antibodies. Dot blot analysis allows for rapid identification of all of the mutant strains that do not up-regulate PA-I in the presence of host stress-derived bacterial signaling compounds, thereby identifying genes that are required for PA-I expression. All assays are preferably performed in triplicate (3 cell densities×5 groups (4 experimental+1 control)×triplicate (3) assays=45 gene arrays).

It is expected that many of the genes that have already been established to play a role in PA-I expression, including genes in the QS and RpoS regulon, will be identified. However, it is expected that new and unanticipated functions for known genes will also be identified. Further, if CyaB or GacS transcripts are increased in response to opioids or IFN-γ, and if Cya B and GacS transposon knockouts do not respond to either opioids or IFN-γ with an increase in PA-I, then the role of these established biosensors as two-component regulators of opioids or IFN-γ signaling to P. aeruginosa will be confirmed. Combining the results of the transcriptome analyses with the functional analyses of the transposon library will permit a determination of whether opioids and IFN-γ activate common membrane biosensors and common downstream genes involved in PA-I expression. It is possible that one or more of the non-peptide opioids diffuses directly into the bacterial cell cytoplasm where it initiates gene activation downstream of the two-component membrane biosensors. If this is the case, then all of the transposon knockout strains encoding membrane proteins are expected to respond with an increase in PA-I and microarray data will demonstrate that levels of transcripts encoding membrane proteins will be unaltered by either opioids or IFN-γ. However, it is possible that membrane biosensors are constitutively expressed and therefore gene expression will not change in response to opioids or IFN-γ. If this is the case, then the entire transposon library will be screened for PA-I expression in response to opioids or IFN-γ, approaches that are feasible given the high-throughput nature of the Dot-Blot technique. Of note here is that gene expressions can be triggered at different times during culturing and can respond to an exogenous compound(s) to varying degrees depending on the concentration of compound. The genomically sequenced strain PAO1 makes abundant PA-I and the anti-PA-I lectin/adhesin antibodies are highly specific.

The data demonstrate that opioid receptor agonists and IFN-γ signal P. aeruginosa to express PA-I mRNA and protein. In addition, these PA-I signaling compounds induce P. aeruginosa to express a more virulent phenotype against the cell in their environment (e.g., epithelium, endothelium). The genes that control PA-I expression are dependent on two key global regulatory systems that activate hundreds of virulence genes in P. aeruginosa. The activation of these interconnected systems of virulence gene regulation are directly influenced by membrane biosensors that recognize elements of host cells and include, but are not limited to, CyaB and GacS, via a hierarchical cascade involving the transcriptional regulators Vfr and Gac A. Genes that are differentially expressed in response to opioids and IFN-γ will be identified using an unbiased transcriptome analysis approach. This approach was chosen instead of pursuing individual candidate genes involved in known pathways of PA-I expression because all previous studies have been performed only at high cell densities and in the absence of any host cell elements

D. Identify the Receptors in P. aeruginosa that Bind Morphine and IFNγ

The data show that a single solubilized membrane protein from P. aeruginosa can be isolated that avidly binds IFN-γ. In addition, morphine also binds to membrane protein fractions. Because antibody is available that specifically recognizes each of IFN-γ and morphine, initial studies are examining the effect of these two BSCs. Using commercial antibodies, the membrane proteins that bind IFN-γ and/or morphine are identified and, optionally, purified. This protein-based approach provides data which complements the experiments described above.

Two approaches available for use in identifying membrane proteins that bind IFN-γ and/or morphine are now described. First, membrane proteins of P. aeruginosa strain PAO1 are solubilized using mild detergents. The binding capacity of solubilized protein fractions for IFN-γ or morphine is then determined using simple ELISA binding assays.) Protein fractions are then immunoprecipitated using the respective antibody and proteins are identified, e.g., by MALDI-MS (matrix-associated laser desorption ionization-time-of-flight mass spectroscopy).

Confirmation of the identity of a binding protein(s) is achieved by determining that a transposon knockout of the gene encoding the candidate protein(s) does not respond to IFN-γ or morphine with an increase in PA-I, using the techniques described herein. In order to confirm the function of candidate proteins showing fidelity in these two analyses, candidate proteins are re-expressed in the corresponding transposon knockout to verify that the PA-I response is re-established. Additionally, receptor antagonists may also be developed.

The data indicate that membrane receptors for morphine and IFN-γ can be identified by identifying proteins from solubilized membranes. A potential limitation using this technique is that morphine could diffuse directly into the bacterial cytoplasm and interact with a downstream target and not a membrane protein. This possibility is consistent with results demonstrating that morphine does not change the transcript profiles of any genes encoding membrane proteins, but the data for IFN-γ disclosed herein is inconsistent with this interpretation. In addition, morphine binding to a solubilized bacterial membrane protein was demonstrated using fluorescent imaging and analysis. Also, there is the possibility that transmembrane proteins or proteins that bind host stress-derived BSCs could be secreted into the culture medium and not be present within bacterial membranes. Examples of such proteins are the bacterial iron binding proteins (enterochelins), which are released by bacteria into the culture medium and then re-enter the bacterial cells. Under such circumstances, the screening of cytosolic fractions and inner and outer membrane preparations are contemplated, along with iterative experiments probing for binding proteins with specific antibodies. Any discordance between the transposon mutant experiments and the proteins purified from bacterial membranes will be reconciled by analyzing IFN-γ-membrane protein or morphine-membrane protein interactions directly using surface plasmon resonance and mass spectrometry.

### Example 16

### The Impact of Host Signaling on Microbial Virulence States

The data demonstrate that PA-I knockout strains (lecA⁻) do not decrease the TEER of cultured epithelial cells. The lethality of strains of P. aeruginosa exposed to opioid agonists and IFN-γ can be defined in vivo using the well-characterized invertebrate Caenorhabditis elegans.

A. The Defect in Cell Barrier Function Induced by P. aeruginosa Exposed to Opioid Agonists and IFN-γ and the Role of PA-I in this Response

One issue is whether opioids or IFN-γ can activate P. aeruginosa to express a lethal phenotype against an epithelium, or endothelium, as judged by an increase in exotoxin A flux across cell monolayers, through the action of its PA-I lectin/adhesin.

To address that issue, MDCK cells or HLMVEC cells are grown to confluence to maintain a stable TEER in transwells. Cells are apically inoculated with P. aeruginosa strain PAO1 (10⁷ cfu/ml) in the presence and absence of varying doses of morphine (about 20 μM), κ agonist (about 80 μM), δ agonist (about 80 μM), or IFN-γ (about 10 μg/ml). To optimize the effect of opioids and IFN-γ on the barrier dysregulating effect of P. aeruginosa against the cells, dose and time response curves are generated. TEER is measured using chopstick electrodes hourly for 8 hours. The apical to basolateral flux of exotoxin A using Alexa-594 labeled exotoxin A is determined in iterative experiments performed at each hourly time point in order to correlate the decrease in TEER to exotoxin A flux for each condition. In selected experiments in which a significant permeability defect to exotoxin A is established, the specific role of PA-I is defined by performing iterative experiments in the presence and absence of 0.3% GalNAc (N-acetylgalactoside) and 0.6% mellibiose, two oligosaccharides that specifically bind to PA-I. Irrelevant sugars (heparin/mannose) are used as negative controls. Iterative studies are also performed using the PA-I transposon knockout (lecA−) mutant to define the specific role of PA-I in strains exposed to opioids and IFN-γ. It is expected that PA-I will be expressed and localized to the microbial pathogen cell surface, where it will be situated in position to interact with host cells, thereby influencing, at a minimum, the cell barrier properties of the cells.

It is expected that opioids and IFN-γ will decrease the TEER of MDCK cells and/or HLMVEC cells. Exotoxin A flux that is increased in cell monolayers with a low TEER will suggest that the opioids and IFN-γ alone can induce a lethal phenotype in P. aeruginosa. If the GalNAc, mellibiose inhibition studies, or the PA-I lectin/adhesin knockout strains, prevent P. aeruginosa from altering TEER and exotoxin A flux across the cell monolayers, then this will indicate that the observed response is PA-I-mediated. If the PA-I knockout mutant strains alter TEER and exotoxin A flux in response to opioids or IFN-γ, then this will indicate that PA-I alone may not be responsible for the virulence of P. aeruginosa against these cells. Data from these studies are directly compared and correlated to worm and mouse lethality studies to determine if these in vitro assays accurately predict a lethal phenotype in vivo, as expected.

### Example 17

### The Roles of Opioid Agonists and IFN-γ on Gut-Derived Sepsis Due to P. aeruginosa as Revealed using Caenorhabditis elegans and Surgically Stressed Mice

The data provide strong evidence that opioid agonists and IFN-γ enhance the virulence of P. aeruginosa in vitro through the action of PA-I. The degree to which opioid agonists and IFN-γ influence the in vivo lethality of P. aeruginosa is, however, unknown. Thus, the ability of opioids and IFN-γ to enhance the in vivo lethality of P. aeruginosa is assessed, e.g., in two complementary animal models.

Wild-type N2 Caenorhabditis elegans worms are grown to the L4 larval stage on normal growth medium (NGM) with E. coli OP50 as a nutrient source. Specialized agar plates are prepared onto which the PA-I-inducing compounds (vehicle (negative control), opioids (morphine, κ and δ agonist), IFN-γ, and C4-HSL (positive control)) will be added and absorbed into the agar as described for ethanol. The ability to embed bioactive compounds into the C. elegans growth agar is well known. Lawns of P. aeruginosa (wild-type PAO1 and PA-I knockout PAO1 (lecA−)) are then grown on solid at agar plates by adding cultures of P. aeruginosa previously grown overnight in liquid media. Worms from the NGM medium are transferred onto the prepared culture dishes and killing dynamics assessed over time at 25° C. Experiments are performed at different doses and re-dosing schedules to establish the optimum conditions under which a killing effect for each of the PA-I-inducing compounds can be demonstrated.

To test the ability of PA-I inducing compounds to enhance the lethality of P. aeruginosa in the established mouse model of gut-derived sepsis, mice are fasted for 24 hours and are subjected to general anesthesia, a 30% surgical hepatectomy, and cecal instillation of 10⁶ cfu/ml of wild-type PAO1 or PAO1 (lecA−) via direct puncture. Dose-response curves for P. aeruginosa in this model have been established and show that 10⁶ cfu/ml of P. aeruginosa induces a 50% mortality rate at 48 hours. In order to demonstrate that opioid agonists or IFN-γ enhance the lethality of P. aeruginosa in this model, varying doses of each are suspended in 1 ml of 0.9% NaCl and injected retrograde into the ileum in order to provide a constant supply of the PA-I-inducing compound for 24 hours. Normal saline alone is used for controls. This maneuver is known to be efficacious in delivering a continuous supply of an exogenous compound to the cecum in this model. Mice are fed water only for the next 24-48 hours and mortality recorded. Mice that appear moribund are sacrificed and the cecal mucosa, liver, and blood are cultured for P. aeruginosa growth on Pseudomonas isolation agar (PIA) in order to quantify bacterial adherence and dissemination patterns. The mice used in the study include two strains (wild-type+ PA-I knockout) and, with a preference for 6 groups of 10 mice per group, a total of 120 mice is suitable.

Increased mortality in worms feeding on lawns of P. aeruginosa in the presence of opioids and/or IFN-γ demonstrates the ability of these compounds to induce a lethal phenotype in this organism against the intestinal epithelium. The demonstration of enhanced killing of worms in these experiments also serves to establish the feasibility and applicability of this model. As disclosed herein, in the absence of PA-I-inducing compounds, C. elegans displays a 50% mortality rate at 80 hours. In testing opioids and/or IFN-γ, or in screening for modulators of PA-I lectin/adhesin activity in general, it should be noted that, following 48 hours of growth and reproduction, worms can reproduce and progeny worms can be indistinguishable from the parent worms and overgrow the plates. If killing dynamics in response to PA-I-inducing compounds are such that observations extend past 48 hours, then use of a temperature sensitive mutant, e.g., C. elegans GLP4 (which does not reproduce at 25° C.), is preferred. Complementary experiments in mice will verify results obtained with worms.

The use of mouse studies to confirm results obtained with C. elegans preferably includes verification that luminally delivered PA-I-inducing compounds are efficacious in up-regulating PA-I as a general measure of enhanced virulence. To control for this possibility, experiments are performed to show that the PA-I-inducing compounds injected into the small bowel enhance PA-I expression in the mouse cecum. One approach involves the use of quantitative RT-PCR for PA-I and exotoxin A on freshly isolated RNA from cecal contents 24 hours following cecal instillation of P. aeruginosa. An alternative approach to delivering opioids and IFN-γ directly into the cecum is to engineer non-pathogenic E. coli strains that produce both morphine and IFN-γ. The feasibility of making recombinant morphine and IFN-γ in E. coli is well known. Mice subjected to a surgical stress (e.g., hepatectomy) are then co-inoculated directly into the cecum with the LD₅₀ dose of P. aeruginosa (approximately 10⁶) and the morphine- and/or IFN-γ-producing E. coli strain. In this manner, P. aeruginosa would be directly exposed to a constant supply of the PA-I -inducing compound such as might naturally occur in vivo. Relevant here is the knowledge in the art that numerous microbial strains (E. coli, Pseudomonas, Candida) naturally produce opioids, especially morphine. In addition, the “microbial soup” typical of a critically ill patient consists of highly pathogenic and resistant strains of bacteria that compete for nutrients in a highly adverse environment. Therefore, not only will the use of morphine- and/or IFN-γ-producing E. coli constitute a feasible alternative approach to obtaining in vivo mouse data, it may also recapitulate actual events in vivo. Finally, C. elegans normally feed on E. coli strains that do not induce mortality. The availability of morphine- and/or IFN-γ-producing E. coli strains could also be used in the C. elegans assays. Others have shown the feasibility of this approach is feasible in mice, as shown by delivering IL-10 into the intestinal mucosa of mice using direct intestinal instillation of bacteria that produce recombinant IL-10 (Steidler et al., Science, 289:1352-5, 2000). The use of the C. elegans assay is expected to result in the rapid identification of therapeutics and prophylactics that modulate expression of a virulence phenotype by microbial pathogens in contact with, or proximity to, a mammal. The virulence phenotype is amenable to assessment using a variety of measures, many of them indirect, e.g., measurement of epithelial cell barrier function.

In addition, characterizations of the roles of known BSCs in expression of microbial virulence phenotypes affecting other cell types, as well as screens for modulators of such physiologic developments, is expected to benefit from the use of the above-described worm and mouse model systems. The virulence of, e.g., P. aeruginosa, towards epithelial cells (intestinal epithelial cells) is expected to provide a sound prediction of the virulence of the same microbe under conditions that are similar or identical, with the exception of the identity of the eukaryotic cells. Thus, the influence of P. aeruginosa on intestinal epithelia is predictive of the influence of P. aeruginosa on other cells of that organism, such as lung epithelia and lung endothelia.

### Example 18

### Use of Knockout Mice to Confirm the Role of BSCs on PA-I Lectin/Adhesin Activity

IFN-γ is a key immune element that actively participates in both the local and systemic clearance of bacteria during acute infection. Animal models have shown that IFN-γ knockout mice have higher mortality rates following infectious challenge at local tissue sites (lung) compared to IFN-γ-sufficient mice in association with diminished ability to clear bacteria. Virtually all of the studies that have assessed the role of IFN-γ on P. aeruginosa infection in vivo have been performed in non-stressed mice where the infectious challenge has been instilled into the lung, and not in stressed mice, such as surgically stressed mice.

The lethality of intestinal P. aeruginosa is tested in IFN-γ knockout mice and wild-type controls (n=10 each group) in an established model of gut-derived sepsis. Mice fasted for 24 hours undergo 30% surgical hepatectomies followed by instillation of 10⁶ cfu/ml of wild type PAO1 into each cecum via direct puncture. Mice are then allowed water only for the remainder of the experiment and mortality is followed for 48 hours. Mice that appear moribund are sacrificed and the cecal mucosa, liver, and blood is quantitatively cultured on Pseudomonas isolation agar (PIA) to determine the rates of bacterial adherence and dissemination. To determine if PA-I expression in P. aeruginosa is attenuated in IFN-γ, a GFP PA-I reporter strain is injected directly into the cecum of mice subjected to a 30% hepatectomy and bacterial strains are recovered 24 hours later to determine fluorescence. The results of these experiments guide the performance of complementary studies using the segmental mesenteric ischemia model. Briefly, the lumena of 10 cm ileal segments subjected to sham ischemia (no clamp), 10 minutes of ischemia, and 10 minutes of reperfusion are perfused with Ringers solution and the timed aliquots of the perfusates are collected from both IFN-γ knockout mice and their wild-type cohorts. Use of the GFP-PA-I reporter strains facilitates the determination of the extent to which each perfusate induces PA-I promoter activity. A suitable number of mice for such studies is 50 mice, divided into live groups with ten mice in each group.

The display of attenuated lethality by P. aeruginosa in IFN-γ knockout mice is consistent with IFN-γ playing a role as a host stress-derived bacterial signaling compound, or protein, during stress (e.g., surgical stress). IFN-γ may be only one of many signals necessary to orchestrate a fully lethal virulence repertoire for P. aeruginosa under the circumstances of catabolic stress, however. It is noted that IFN-γ knockout mice subjected to hepatectomy may develop an overcompensated and excessive inflammatory response to intestinal P. aeruginosa, resulting in increased mortality that is based more on immune response than enhanced microbial virulence. Tissue and blood culture results from these studies are used to determine whether mortality is due, in part, to such overcompensation. An alternative approach to distinguish between these possibilities is to perform studies in IFN-γ knockout mice and their matched wild-type cohorts (with and without surgical hepatectomy) to determine if there is a mortality difference when groups of mice are systemically inoculated (e.g., intraperitoneal, intravenous, lung instillation) with P. aeruginosa.

### Example 19

### Screens for Stress-Induced Bacterial Signaling Compounds

The data disclosed herein establish that i) filtered luminal contents from the cecum of mice subjected to hepatectomy, or from the small bowel lumen of intestinal segments subjected to mesenteric arterial occlusion, induce a strong signal in P. aeruginosa to express PA-I; and ii) media and membrane preparations from hypoxic or heat-shocked Caco-2 cells induce PA-I expression.

A. Stress-Derived BSCs that are Present in the Media of Caco-2 Cells Exposed to Ischemia and Heat Shock Stress and that Induce PA-I Expression in P. aeruginosa

Intestinal epithelial hypoxia is a common consequence of critical illness following surgical stress and is often an inadvertent consequence of its treatment. In addition, hyperthermia often develops during the acute stress response to injury and infection. Disclosed herein are data demonstrating that hypoxic or hyperthermic stress to cultured intestinal epithelial cells (Caco-2) causes the release of soluble PA-I-inducing compounds into the cell culture medium. This example discloses the isolation and identification of PA-I-inducing compounds that are released by Caco-2 cells exposed to hypoxia and hyperthermic stress.

Two sets of experiments are preferably performed. In the first set of experiments, Caco-2 cells grown to confluence in cell culture plates (150 cm²) are exposed to either normoxia (21% O₂) or hypoxia (0.3% O₂ for 2 hours followed by 1 hour of normoxic recovery). In the second set of experiments Caco-2 cells are exposed to normothermic (37° C.) or hyperthermic (immersed in water bath at 42° C. for 23 minutes followed by 3 hours recovery) conditions. Paired samples from each set of experiments are then processed to identify the specific host stress-derived bacterial signaling compound(s) using GFP-PA-I reporter strains as a detection system. Media from Caco-2 cells is collected, filtered through a 0.22 μm filter (Millipore) and separated by molecular weight using centricones with MW cutoffs of 3, 10, 30, 50, and 100 KDa (<3, 3-10, 10-30, 30-50, 50-100, 100 KDa). All fractions are preferably tested in 96 well plates to determine fractions that activate PA-I expression using PA-I GFP reporter strains. Two preferred approaches are contemplated for use in identifying the proteins that activate PA-I in the stress-conditioned media (hypoxia, hyperthermia). The first approach subjects bioactive fractions (i.e., those that induce PA-I), and their molecular weight-matched control fractions (non-PA-I-inducing), to MALDI-MS analysis. Spectra from the control media fractions are compared to the fractions of stress-conditioned media to determine the appearance of possible protein molecular ions present only in the samples that induce PA-I. This approach will allow subtraction of proteins that are present in both non-PA-I-inducing and PA-I-inducing fractions. In order to separate the molecular ion protein peaks that are present only in the PA-I-inducing fractions, bioactive fractions are loaded onto an HPLC equipped with a Vydac C4 column. Eluted samples are collected as fractions and individual fractions are tested for the ability to induce PA-I expression using the GPF-PA-I reporter strain. Proteins are then further separated, preferably by MW, hydrophobicity, and charge using stepwise, well-controlled, physico-chemical separation techniques in the HPLC system. Samples pre-fractionated in this manner should simplify the observed mass spectra and increase the likelihood of observing any putative protein(s) that induce PA-I expression. For any such proteins, identification using bottom-up proteomics techniques is performed.

An alternative to the use of molecular ion spectra, suitable in studies presenting highly complex spectra, is the classical approach for protein purification using conventional techniques such as ion exchange, hydrophobic, size exclusion, and/or affinity chromatography. Purification of host stress-derived BSCs is preferably assessed using the GFP-PA-I reporter strain.

For protein identification, protein-containing fractions are digested by using trypsin and digested fractions are analyzed with a LC/MSD XCT ion trap mass spectrometer system (Agilent Technologies, Santa Clara. CA). Data analysis for the data from the mass spectrometer is carried out using the SpectrumMill software platform (Agilent Technologies, Santa Clara, Calif.). Confirmation of the ability of identified proteins to induce PA-I expression is conveniently achieved in the PA-I:EGFP reporter strain by measuring fluorescence, and in P. aeruginosa strain PAO1 by immunoblot analysis.

Two protein fractions from Caco-2 cells that induce PA-I expression have been identified. Identification of specific active proteins (i.e., epithelial cell-derived PA-f signaling proteins) within the fraction(s) is achieved using any known technique, and preferably using a proteomics facility such as The University of Chicago proteomics facility. Many of these proteins may originate from the cell membranes themselves, since the most potent induction of PA-I expression occurs following contact with an epithelial cell membrane. In addition to protein identification, antibodies specifically recognizing such proteins are contemplated for such uses as cellular (e.g., Caco 2) localization studies. Although there are more classical approaches to protein identification, mass spectrometry is the most cost effective and rapid approach. For non-proteinaceous PA-I inducing compounds, lipid assays are contemplated that involve adjusting fraction pH to 3.5, followed by HPLC using, e.g., a Sep-Pak C₁₈ column. Eluted samples are trapped on a fraction collector, evaporated to dryness, and re-suspended in PBS for PA-I reporter assays. The structure of the active compound is preferably identified with IT/LC/MS/MS. For bacterial signaling compounds that are neither protein nor lipid, relevant fractions are resolved by IT/LC/MS/MS using a C₁₈ column and a quadrapole-timc of flight mass spectrometer and NMR. Individual compounds are determined by their mass-fragmentation spectra, isolated, and tested for PA-I inducing activity using GFP reporter strains. Alternative approaches, such as 2D-SDS-PAGE electrophoresis for protein separation and TLC for non-protein separation, are also contemplated. Proteins separated by 2D-SDS-PAGE are typically transferred to a polyvinylidene difluoride transfer protein membrane for automated Edman degradation N-terminal sequence determination using an ABI 477A protein sequencer (Applied Biosystcms). Protein identification is further facilitated by sequence comparison to database(s) sequence(s).

In addition to the foregoing screens for modulators, the invention contemplates any assay for a modulator of the expression of a virulence phenotype by a microbe in association with, or proximity to, a mammal such as a human. In particular, the invention comprehends a wide variety of assays for modulators of, e.g., eukaryotic cell barrier function, such as epithelial and endothelial cell barrier function (e.g., epithelial cells of the intestine, epithelial and endothelial cells of the lung, and the like). The invention further comprehends numerous assays for modulators of PA-I lectin/adhesin activity, whether due to a modulation of the specific activity of PA-I or a modulation of the expression of PA-I of constant specific activity, or both. In general, the invention contemplates any assay known in the art as useful for identifying compounds and/or compositions having at least one of the above-described characteristics.

### Example 20

### Miscellaneous Methods

A. Screens for PA-I Modulators using a PA-I Reporter Construct

Media from Caco-2 cells exposed to either hypoxia or heat shock stress induced PA-I expression in P. aeruginosa. Candidate PA-I inducer compounds that are released into the extracellular milieu following epithelial stress include ATP, lactate, cAMP, cytokines, and heat shock proteins.

The aforementioned candidate modulators, and other candidate modulators found in properly conditioned media, are identified using screening methods that constitute another aspect of the invention. Screens for such modulators are conveniently conducted in 96-well plates that contain the GFP-PA-I reporter strain PA27853/PLL-EGFP (see Example 1). The reporter strain is exposed to varying concentrations of candidate host stress BSCs including, but not limited to, heat shock proteins (HSP 25, 72, 90, 110), extracellular nucleosides and nucleotides (adenosine, ATP, cAMP) and cytokines (IL-1-18). Agents are added to the wells and dynamic assessment of bacterial fluorescence is earned out over 12 hours. Positive results are preferably verified by Western blot analysis of PA-I expression. For proteins that induce a PA-I response, the invention further comprehends assays to identify the receptors on P. aeruginosa to which such proteins bind. In one embodiment of this aspect of the invention, the identified protein inducer of PA-I activity is used as a probe to screen, e.g., a comprehensive library of P. aeruginosa by dot blot analysis. Confirmation of the screen results is available by assaying the protein-binding capacity of a lysate from a clone from a P. aeruginosa transposon library in which the relevant coding region has been disrupted by insertional mactivation.

Identified modulators are then subjected to additional in vitro and in vivo virulence assays to refine the understanding of the role in virulence expression played by such modulators.

### B. Caco-2 and MDCK Cell Culture, Measurement of TEER and Exotoxin A Flux.

Caco-2 cells and MDCK cells are well-differentiated epithelial cell lines that maintain a stable TEER when grown in confluent monolayer. Apical to basolateral exotoxin A flux across monolayers is assessed with Alexa 594 labeled exotoxin A using standard flux measurements.

### C. Bacterial Strains

P. aeruginosa strain PAO1 was obtained from the University of Washington Genome Center and is preferably used in the procedures disclosed herein, where appropriate.

D. Caenorhabditis elegans Assays.

Use of the nematode to assay for the lethality of P. aeruginosa is accomplished using standard protocols, as described herein.

### E. Antibodies.

Antibodies to PA-I are generated using conventional techniques. Preferably, such antibodies are purified by affinity chromatography. IFN-γ and morphine antibodies are commercially available.

### F. Dot Blot Assays for Membrane Binding.

ImmunoDot Blot assays for the detection of bacterial proteins in large matrix systems have been described elsewhere (Bolin et al., J Clin Microbiol, 33:381-4, 1995, incorporated herein by reference) and are known in the art. The technique has been validated as highly sensitive and accurate.

### G. Transcriptome Analysis of Bacterial Strain PAO1.

RNA is isolated from bacterial cultures exposed to opioids and/or IFN-γ as described herein at optical densities of 0.5, 1.0, 2.0. Between 1×10⁹ and 2×10⁹ cells are then mixed with RNA Protect Bacteria reagent (Qiagen) and treated as recommended by the manufacturer's mechanical disruption and lysis protocol. RNA is purified by using RNeasy mini columns (Qiagen), including the on-column DNase 1 digestion described by the manufacturer. In addition, the eluted RNA is preferably treated for 1 hour at 37° C. with DNase I (0.1 Upper μg of RNA). DNase I is then removed by using DNA-Free (Ambion) or by RNeasy column purification. RNA integrity is monitored by agarose gel electrophoresis of glyoxylated samples. Further sample preparation and processing of the P. aeruginosa GeneChip genome arrays are then done as described by the manufacturer (Affymetrix). For cDNA synthesis 12 μg of purified RNA is preferably combined with semirandom hexamer primers with an average G+C content of 75%, and Superscript II reverse transcriptase (Life Technologies). Control RNAs from yeast, Arabidopsis, and Bacillus subtilis genes are added to the reaction mixtures to monitor assay performance. Probes for these transcripts are tiled on the GeneChip arrays. RNA is removed from the PCR mixtures by alkaline hydrolysis. The cDNA synthesis products are purified and fragmented by brief incubation with DNase 1, and the 3′ termini of the fragmentation products are labeled with biotin-ddUTP. Fragmented and labeled cDNAis hybridized to an array by overnight incubation at 50° C. Washing, staining, and scanning of microarrays is performed with an Affymetrix fluidics station.

### H. Analysis of Expression Profiling.

The Affymetrix Microarray Software suite (MAS) (version 5.0) is a suitable software choice for determining transcript levels and whether there are differences in transcript levels when different samples are compared. Affymetrix scaling is used to normalize data from different arrays. A scale factor is derived from the mean signal of all of the probe sets on an array and a user-defined target signal. The signal from each individual probe set is multiplied by this scale factor. For any given array, between 18 and 28% of the mRNAs are considered absent by MAS, indicating that the corresponding genes are not expressed at levels above background levels. Furthermore, it is known in the art that the average changes in control transcript intensities are less than twofold for any comparison of array data. This indicates that the efficiency of cDNA synthesis and labeling is similar from sample to sample. For comparative analyses, the log₂ ratio for absolute transcript signals obtained from a given pair of arrays will be calculated by using MAS. A statistical algorithm of the software is also assigned a change call for each transcript pair, which indicates whether the level of a transcript is significantly increased, decreased, or not changed compared to the level for the baseline sample. The baseline samples are those derived from cultures of P. aeruginosa PAO-1 without any added opioids or IFN-γ. Graphical analyses of the signal log ratios from each experiment (any pair of arrays) is performed to display a normal distribution with a mean very close to zero (no change). Among the transcripts with significant increases or decreases compared to the baseline in one or more samples, those that showed at least a 2.5-fold change are subjected to further analysis. For cluster analyses and transcript pattern analyses, GeneSpring software (Silicon Genetics, Redwood City, Calif.) is contemplated as a suitable choice. The fold change values for each gene will be normalized independently by defining the half-maximal value for the gene as 1 and representing all other values as a ratio that includes that value. This scaling procedure will allow direct visual comparison of gene expression patterns within an experiment, as well as between experiments. GeneSpring is also contemplated for use in sorting genes according to the P. aeruginosa genome project.

I. Solubilization of Non-Denatured and Denatured Membrane Proteins Fractions from P. aeruginosa.

P. aeruginosa cells are washed with PBS and re-suspended in PBS containing a protein inhibitor cocktail. For preparation of membrane fractions, P. aeruginosa cells are disrupted by French pressure and centrifuged at 10000 g×30 minutes to eliminate debris. The supernatant is recentrifuged at 50000 g×60 minutes. The pellet is solubilized in 4% CHAPS at 37° C. for 3 hours. After being recentrifuged at 50000 g×60 minutes, the supernatant is spun through a 100K centricone and dialyzed against PBS. The binding capacity of the solubilized protein to γ-IFN is confirmed by ELISA binding assay.

### J. Statistical Analysis and Protein-Protein Interactions.

For statistical analysis, all data are preferably loaded into the SigmaStat platform software and appropriate tests applied. Protein-protein interaction studies are performed using conventional protocols, as would be known in the art.

### K. MALDI-MS Analysis.

Samples (0.5 μL) are mixed with an equal volume of a 5 mg/mL solution of α-cyanohydroxycinnamic acid in 30% acetonitrile in water with 0.1% TFA and are then manually spotted onto a 192 spot target plate (Applied Biosystems, Foster City, Calif.). The plate is inserted into a 4700 MALDI TOF/TOF (Applied Biosystems, Foster City, Calif.) operated in linear mode. Samples are desorbed by a 200 Hz YAG laser. The acquisition program is set to acquire summed spectrum (200-1000) shots across the range 5000 to 100000 Thompsons.

L. Digestion of a Protein-Containing Fraction by using Trypsin to Prepare for Protein Identification.

The protein extract sample is diluted in 50 mM ammonium carbonate buffer, pH 8.5, containing 0.1% Rapigest SF acid-labile detergent (Waters Corp, Millford, Mass.). The sample is heated to 100° C. for 10 minutes to completely denature the proteins. Ten μL of 10 mM TCEP is added to reduce disulfide bonds and the sample is incubated for 10 minutes at 37° C. The pooled sample is digested with Lys-C (12.5 ng/μL) at a mass ratio of 1:100 for 3 hours at 37° C. and then digested with trypsin (12.5 ng/μl) at a mass ratio of 1:50 (trypsin:protein) for 3 hours at 37° C. Digestion is halted by adding PMSF to final concentration of 1 mM. After digestion. 10 μL of TFA is added to the solution and the sample is incubated for 45 minutes at 37° C. to destroy the acid-labile Rapigest detergent.

### M. LC/MSD XCT Ion Trap Mass Spectrometry Analysis.

A digested protein sample is injected (10 μL) onto an HPLC (Agilent Technologies 1100) containing a C18 trapping column (Agilent Technologies, Santa Clara, Calif.) containing Zorbax 300SB-C18 (5×0.3 mm). The column valve is switched to its secondary position 5 minutes after injection and the trapped peptides are then eluted onto a 75 μm id Zorbax Stablebond (300 A pore) column and chromatographed using a binary solvent system consisting of A: 0.1% formic acid and 5% acetonitrile and B: 0.1% formic acid and 100% acetonitrile at a flow rate of 300 mL/minute. A gradient is run from 15% B to 55% B over 60 minutes on a reversed-phase column (75 μm id Zorbax Stablebond (300 A pore)), and the eluting peptides are sprayed into a LC/MSD XCT ion trap mass spectrometer system (Agilent Technologies, Santa Clara, Calif.), equipped with an orthogonal nanospray ESI interface. The mass spectrometer is operated in positive ion mode with the trap set to data-dependent auto-MS/MS acquisition mode. Source conditions are: Vcap −4500V, drying gas flow 8 L/minute, drying gas temperature 230° C. and CapEx 65V. The instrument is set to complete a mass scan from 400-2200 Thompsons in one second. Peaks eluting from the LC column that have ions above 100,000 arbitrary intensity units trigger the ion trap to isolate the ion and perform an MS/MS experiment scan after the MS full scan. The instrument's dynamic ion exclusion filter is set to allow the instrument to record up to 2 MS/MS spectra for each detected ion to maximize the acquisition of qualitative data from peptides (by preventing high abundance peptides from dominating the subsequent MS/MS experiments) and the excitation energy is set to “smart frag” mode to insure the generation of useful product ion spectra from all peptides detected. Data files that result are then transferred to a file server for subsequent data reduction.

N. The Mass Spectrometer Data Analysis with the SpectrumMill Software Platform.

SpectrurnMill is derived from the MS-Tag software package originally developed by Karl Clauser et al., Anal Chem, 71:2871-82, 1999 and is contemplated as a suitable software platform. Raw data is extracted from the MS data files using the data extractor module and the data is then subjected to protein library search and de novo spectral interpretation by the Sherenga module (Dancik et al., J Comput Biol, 6:327-42, 1999). SpectrumMill is designed to minimize spurious identifications obtained from the MS/MS spectra of peptides by careful filtering and grouping of related MS and MS/MS data during extraction from the raw data file. The library searching and de novo interpretation identify the detected proteins form the individual peptides. The results for all proteins detected are collected and listed by protein name, detected peptide sequence(s), and search score. The reports are exported to an Excel spreadsheet file for inclusion in a result database. In addition, data extracted from the raw data files from the ion trap are preferably submitted to the Mascot (MatrixScience Inc, London, UK) search program and searched against both the NCBI non-redundant protein database and the SWISSPROT protein database. The identifications from these two systems are correlated to arrive at a final consensus list of identified proteins.

### O. Separation of Lipid Fractions on HPLC System.

Fractions are pH adjusted to 3.5, and run across a Sep-Pak C₁₈ column on a HPLC system (Millipore corp., Milford, Mass.). The columns are washed with ddH₂O, and compounds are eluted with increasingly polar mobile phases (hexane-methyl formate-methanol). Fractions are concentrated under a stream of nitrogen and reconstituted in either 1 ml PBS (for PA-I reporter assay) or 100 μl of methanol (for UV/HPLC). Active fractions from Sep-Pak are preferably further resolved by a C₁₈ reversed-phase HPLC column (150 mm×5 mm, Phenomenex, Torrance, Calif.) with acidified (0.1% acetic acid) Methanol:H₂O (60:40 vol/vol) at 1 ml/minute on a 1050 series HPLC using ChemStation™ software (Hewlett Packard, Palo Alto, Calif.).

### Example 21

### Hypoxia-Induced PA-Lectin Adhesin Expression

The aim of the study described in this Example was to determine whether intestinal epithelial hypoxia, a common response to surgical stress, could activate PA-I expression. Because splanchnic vasosconstriction and intestinal epithelial hypoxia are a common consequence of surgical injury, the aim of the experiments described herein was to determine the specific role of the intestinal epithelium in signaling to P. aeruginosa by examining the effect of epithelial cell hypoxia and reoxygenation on PA-I expression. A fusion construct was generated to express green fluorescent protein downstream of the PA-I gene, serving as a stable reporter strain for PA-I expression in P. aeruginosa, as described in Example 1. Polarized Caco-2 monolayers were exposed to ambient hypoxia (0.1-0.3% CV) for 1 hour, with or without a recovery period of normoxia (21% O₂) for 2 hours, and then inoculated with P. aeruginosa containing the PA-I reporter construct. Hypoxic Caco-2 monolayers caused a significant increase in PA-I promoter activity relative to normoxic monolayers (165% at 1 h; P< 0.001). Similar activation of PA-I was also induced by cell-> free apical, but not basal, media from hypoxic Caco-2 monolayers. PA-I promoter activation was preferentially enhanced in bacterial cells that physically interacted with hypoxic epithelia. As shown below, the virulence circuitry of P. aeruginosa is activated by both soluble and contact-mediated elements of the intestinal epithelium during hypoxia and normoxic recovery.

### A. Human Epithelial Cells.

Caco-2_{BBe} cells expressing SGLT1 were maintained in DMEM with 25 mM glucose (high-glucose DMEM) with 10% fetal calf serum, 15 mM HEPES, pH 7.4, and 0.25 mg/ml geneticin, as previously described (Viswanathan et al., Adv Drug Delivery Res 56: 727-762, 2004, incorporated herein by reference). Caco-2 cells were plated on 0.33-cm² collagen-coated, 0.4-μm pore size polycarbonate membrane Transwell supports (Corning-Costar, Acton, Mass.) for 20 days, and media were replaced with identical media without geneticin at least 24 hours before bacterial inoculation.

B. GFP Fusion Constructs of Wild-Type P. aeruginosa.

P. aeruginosa (ATCC-27853, American Type Culture Collection, Manassas, Va.) was transformed with the plasmid pUCP24/PLL-EGFP. This construct harbors a PA27853 chromosomal DNA fragment containing an upstream regulatory region of PA-I followed by the entire PA-I gene fused at the COOH terminal with an enhanced green fluorescent protein (EGFP) gene excised from the pBI-EGFP plasmid (Clontech, Palo Alto, Calif.). Expression of the PA-I lectin was detected by fluorescence microscopy and fluorimetry of this reporter strain as previously described (Wu et al., Ann Surg 238: 754-764, 2003, incorporated herein by reference).

### C. Dynamic Fluorimetry.

Caco-2 cells were grown to confluence on collagen-coated 96-well fluorimetry plates (Becton Dickinson Labware, Bedford, Mass.) and maintained in a 37° C. incubator with 5% CO₂ and 21% O₂. The day before experiments, media were removed and replaced with 150 μl of antibiotic-free media. Three experimental conditions were created using a modification of the methods previously described by Xu et al., J Trauma 46: 280-285, 1999, incorporated herein by reference. In control conditions, Caco-2 cells were maintained in a 5% CO₂-21% O₂ incubator for 2 hours. Hypoxic conditions were achieved by placing Caco-2 cells in a humidified hypoxia chamber at 37° C. with 5% CCV95% N₂ for 2 hours. Measured O₂ in the chambers varied between 0.1 and 0.3%. To simulate a reperfusion or reoxygenation state (normoxic recovery), after 2 hours of Caco-2 cell hypoxia, hypoxic media were completely replaced with fresh, normoxic HDMEM media, and the cells were allowed to recover under normoxia (37° C., 5% CO₂-21% O₂) for 2 hours before bacterial inoculation. The fluorescent reporter strain PA27853/PLL-EGFP was next added to the three groups of Caco-2 cells. Bacteria were cultured overnight in Luria-Bertani broth containing 20 μg/ml gentamicin at 37° C. under shaking conditions. After about 12 hours of growth, 50 μl of the bacterial suspension were added to the 96-well plates of Caco-2 cells. Care was taken to ensure that all bacterial samples were cultured for identical periods of time and that wells contained equal cell densities. Fluorescence was tracked immediately following bacterial inoculation and then hourly thereafter up to 3 hours using a 96-well microplate fluorimeter (Synergy HT, Biotek, Winooski, Vt.). Plates were maintained in standard incubators at 37° C. with 5% CO₂-21% O₂ between all measurements. Fluorescence values were calculated as follows: % control=100×(RFUxₜ₌ₙ−RFUxₜ₌₀)/(RFUcₜ₌ₙ−RFUcₜ₌₀), where RFUx refers to the hypoxic or normoxic recovery groups and RFUc refers to the control at time n.

D. Exposure of Bacteria to Filtered Media from Caco-2 Cells and Potential PA-I-Inducing Candidate Molecules.

In this set of experiments, reiterative conditions of control, hypoxia, and normoxic recovery (i.e., reperfusion/reoxygenation) were created in 96-well plates containing confluent Caco-2 cells. Media from all wells were then collected and passed through a 0.22-μm filter and stored on ice. Ninety-six-well fluorimetry plates without Caco-2 cells (Costar 3631, Corning, Corning, N.Y.) were then prepared by adding a 20-μl bacterial suspension containing overnight growing cultures of PA27853/PLL-EGFP. Media from the three experimental groups were then added to the wells, and fluorescence was assessed over 5 hours, with plates maintained at 37° C. with continuous orbital shaking (100 rpm) between measurements. To screen for potential PA-I-inducing compounds that might be present in the media of hypoxic Caco-2 cell media, purified adenosine, D-lactate, and L-lactate (Sigma-Aldrich, St. Louis, Mo.) were added to wells containing HDAIEM across a range of physiologically relevant dosages, which were then tested as described above.

### E. Fluorescence Microscopy.

To visually correlate results from the above experiments to the spatiotemporal effects of PA27853/PLLEGFP on hypoxic Caco-2 cells, cells were grown to confluence on Bioptechs dishes (Bioptechs, Butler, PA) and exposed to 2 hours of hypoxia followed by inoculation with PA27853/PLL-EGFP. Experiments were performed on a 37° C. microscopy stage and visualized using an inverted fluorescence microscope (Axiovert 100, Carl Zeiss, Thornwood, N.Y.). Z-stacks were collected every 30 minutes for 3 hours. Images were analyzed for bacterial distribution using ImageJ graphics analysis software (Version 1.31, National Institutes of Health, Bethesda, Md.).

F. Caco-2 Cell Barrier Function During Hypoxia and Normoxic Recovery in the Presence of P. aeruginosa or Purified PA-I.

Caco-2 monolayer transepithelial electrical resistance (TEER), a measure of general cell (not just epithelia) barrier function, was assessed using agar bridges and Ag-AgCl-calomel electrodes and a voltage clamp (University of Iowa Bioengineering, Iowa City, Iowa) as previously described (Turner et al., Am J Physiol Cell Physiol 273: C1378-C1385, 1997, incorporate herein by reference). TEER was calculated using Ohm's law. Fluid resistance was subtracted from all values (Id.). Two microliters of overnight cultures of PA27853 normalized to cell density or 50 μg of purified PA-1 (Sigma-Aldrich) were added to the apical chamber of the Caco-2 cell transwells following exposure to hypoxia and normoxic recovery as detailed above. Caco-2 cell TEER was assessed every hour, and cells were maintained at 37° C. with 5% CO₂-21% O₂ throughout the experiment. To determine the effect of PA27853 on the barrier function of Caco-2 cells under conditions of sustained hypoxia, reiterative experiments were performed under continuous hypoxia (37° C., 5% CO₂-95% N₂), in which TEER measurements were made every hour for 7 hours within the hypoxic chamber using an EVOM resistance measurement apparatus (World Precision Instruments, Sarasota, Fla.).

### G. Northern Blot Analysis.

In selected experiments, PA-I expression was confirmed using Northern blot analyses as previously described (Wu et al., Ann Surg, 238:754-764, 203, incorporated herein by reference).

### H. Statistical Analysis.

Data were analyzed, and statistical significance was determined using Prism 4.0 (GraphPad Software, San Diego, Calif.). Statistical significance was defined as P< 0.05 by Student's t-test or two-way ANOVA, as appropriate.

The results of these experiments showed that PA27853/PLL-EGFP P. aeruginosa responded to the environment of Caco-2 cell hypoxia and normoxic recovery with enhanced fluorescence. To determine whether the green fluorescent protein (GFP) reporter strain PA27853/PLL-EGFP would display increased PA-I promoter activity when added to Caco-2 cells exposed to hypoxia (2 hours at <0.3% O2) and normoxic recovery (hypoxia followed by 2 hours of recovery in normoxic conditions), reporter strains were added to the media of Caco-2 cells exposed to the two conditions. GFP reporter strains demonstrated significantly more PA-I promoter activity, as measured by fluorescence, within 1 hour of incubation with Caco-2 cells exposed to either hypoxia or normoxic recovery. The media pH in all experimental conditions was measured at all time points and demonstrated no significant difference among control, hypoxia, and normoxic recovery groups because all media were buffered. However, to show that the pH of media did not influence fluorescence in PA27853/PLL-EGFP, strains were incubated in media at pH 6.5, 7.4, and 7.7. The percent change in fluorescence was not different among groups (6.5=106±10, 7.4=100±12, 7.7=112±12; P=not significant). Similarly, to rule out an effect of hypercarbia or hypoxia alone on PA-I promoter activity in our reporter strains, strains were subjected to hypoxia (0.1% for 2 hours) and hypercarbia (80% CO₂ for 2 hours). No difference in fluorescence was observed between groups. Taken together, these findings demonstrate that media from Caco-2 cells exposed to hypoxia with or without normoxic recovery activate PA-I promoter activity.

To determine whether epithelial cell contact contributes to the expression of GFP in our PA-I reporter strain, Caco-2 cells were imaged by fluorescence microscopy following exposure to hypoxia and apical inoculation with PA27S53/PLL-EGFP. Fluorescence imaging demonstrated that PA27853/PLL-EGFP exposed to hypoxic Caco-2 monolayers was markedly more fluorescent than bacteria exposed to normoxic monolayers at the 120-minute time point. Multiple images of the bacterial/Caco-2 cell co-culture demonstrated that more bacteria were located near or within the plane of the cell monolayers exposed to hypoxia than in nonhypoxic cells. Quantitative analysis of multiple microscopy images revealed an average of 658±78 bacteria/high-powered field at the level of the surface of hypoxic epithelia, whereas no bacteria were seen in plane-matched controls (P<0.001).

To determine whether soluble compounds released into the media in response to Caco-2 cell hypoxia were capable of activating PA-I expression independent of bacterial contact with the epithelium, the ability of media from hypoxic Caco-2 cell cultures to enhance fluorescence in our reporter strain was tested. PA27853/PLL-EGFP bacteria exposed to filtered media from Caco-2 cells exposed to hypoxia and normoxic recovery developed a significant enhancement of fluorescence that appeared greatest at the 5-hour time point (control: 3.7%±SD 3.9; hypoxia: 12.6%±SD 5.8; normoxic recovery: 13.1%±SD 3.9; P< 0.001 by 2-way repeated measures ANOVA). Results were confirmed by Northern blot analysis. To determine whether this paracrine factor was isolated to the apical or basolateral compartments, reiterative experiments were performed in which isolated media from the basolateral and apical compartments of hypoxic monolayers, as well as mixtures of apical and basolateral media, were added to wells containing the GFP-PA-I reporter strain PA27853/PLL-EFGP. Only those bacteria exposed to hypoxic media from the apical chamber or hypoxic mixed media showed a statistically significant increase over controls (P < 0.05).

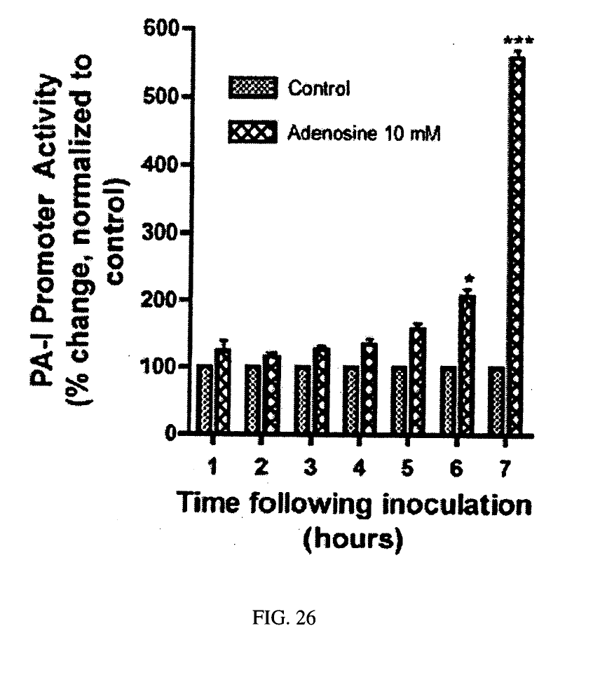

To determine whether candidate compounds specifically released by hypoxic Caco-2 cells could induce the expression of PA-I, the effect of D-lactate, L-lactate, and adenosine was tested in our GFP-PA-I reporter strains. D- and L-lactate had no effect on PA-I promoter activity; however, PLL/PA27853 responded with enhanced fluorescence to 10 mM adenosine (FIG. 26), raising the possibility that adenosine released by hypoxic Caco-2 cells could be the putative mediator of the increased PA-I response observed in the above studies. However, the time required for upregulation of PA-I expression was longer than that observed in response to hypoxic cell media, suggesting that other factors may be involved in the signaling pathway.

To determine whether conditions of hypoxia and normoxic recovery enhance or attenuate the barrier-dysregulating properties of PA27853 against Caco-2 cells, TEER was measured in Caco-2 cells apically inoculated with either PA27853 or purified PA-I following exposure to hypoxia and normoxic recovery. Despite the ability of media from hypoxic and reoxygenated Caco-2 cells to increase the expression of PA-I in P. aeruginosa, the TEER of Caco-2 cells exposed to these conditions were unchanged in response to a P. aeruginosa while Caco-2 cells exposed to purified PA-I exhibited an attenuated drop in TEER compared with normoxic cells (P< 0.05).

To determine whether Caco-2 cells exposed to sustained hypoxia could resist the barrier-dysregulating effect of PA27853, the TEER of Caco-2 cells apically inoculated with PA27853 in an environment of sustained hypoxia was measured. Caco-2 cells maintained TEER equal to hypoxic Caco-2 cells without bacteria and completely resisted the predicted decrease in TEER at the 7-hour time point. That Caco-2 cells partially resist the barrier-dysregulating effect of strains of PA27853 despite increased PA-I expression could be explained by previous observations suggesting that epithelial cells normally respond to hypoxia with an enhancement of local mucosal defense proteins and barrier function.

To determine whether the normoxic Caco-2 cells could be induced to increase their resistance to barrier dysregulation by P. aeruginosa through signals present in hypoxic cell media, the apical and basolateral media of normoxic Caco-2 cells was exchanged with filtered media from the apical and basolateral compartments of hypoxic Caco-2 cells and the barrier function of these cells was tested when apically inoculated with P. aeruginosa. Normoxic Caco-2 cells exposed to media from hypoxic epithelia displayed a prolonged resistance to barrier dysregulation induced by P. aeruginosa, indicating that normoxic epithelia may be activated to enhance their barrier function in the presence of soluble mediators produced during hypoxia.

Although P. aeruginosa is not considered to be an intestinal pathogen in the classic sense, it induces one of the most rapid and profound decreases in intestinal epithelial TEER of any bacterium reported to date. In both Caco-2 and T-84 cells, P. aeruginosa (PA27853) can induce an 80% decrease in TEER within 4 hours following its apical inoculation. If defined by this criterion alone, P. aeruginosa is among the most pathogenic organisms to the intestinal epithelium yet described. The observation that as many as 5% of the normal population harbor this pathogen within their intestinal tracts, coupled with animal studies demonstrating that control mice do not develop any symptoms of infection following the direct introduction of large quantities of P. aeruginosa into the cecum, indicate that this organism behaves like a classic opportunist, switching virulence genes on and off in response to selected environmental cues. Environmental cues such as pH, redox state, and nutrient composition can activate virulence gene expression in bacteria through a variety of membrane-bound biosensor kinases, to which is added bacterial signaling compounds that are released by host cells following physiological or ischemic stress.

To demonstrate that bacteria sense and respond directly to host cells, the PA-I lectin/adhesin of P. aeruginosa was used as a reporter gene. The PA-I lectin is under tight regulatory control of two key systems of virulence gene regulation in P. aeruginosa: the quorum-sensing signaling system and the alternative sigma factor RpoS. The quorum-sensing signaling system and RpoS are interconnected systems of virulence gene regulation in P. aeruginosa that control the expression of hundreds of virulence genes in this pathogen. Because PA-I expression is dependent on the function of both quorum sensing and RpoS, it serves as a relevant biological readout for generalized virulence gene activation in P. aeruginosa. The finding that soluble elements of intestinal epithelial cells, and in particular adenosine, can activate PA-I expression, indicates that specific host cell-derived compounds are released that signal colonizing pathogens such as P. aeruginosa that its host is weak and susceptible. That adenosine alone can activate PA-I expression is an important finding given that adenosine is released and can accumulate in the extracellular milieu of hypoxic tissues at high concentrations. During active intestinal inflammation, 5′-AMP derived from migrating polymorphonuclear leukocytes is converted to adenosine by the apical surface epithelium of the intestine. Strohmeier et al., J Clin Invest 99: 2588-2601, 1997, have demonstrated that under normal conditions, the human intestinal epithelial cell line T-84 can convert substantial amounts of 5′-AMP that accumulate to as much as 5 mM adenosine in the apical media within 30 minutes. Although in the present study, activation of PA-I promoter activity in P. aeruginosa required what appeared to be an unphysiological dose of adenosine, the precise concentration of adenosine to which P. aeruginosa might be exposed within the intestinal tract during prolonged hypoxia and reoxygenation is unknown. In addition, adenosine exposure required 6 hours before PA-I promoter activity was observed, whereas with hypoxic media PA-I promoter activity was observed at 4 hours. Without wishing to be bound by theory, an opportunistic organism like P. aeruginosa may require an inordinately potent and prolonged host-derived signal for it to invest the resources and energy required to mount a toxic offensive against the intestinal epithelium or other host cells. Under such circumstances. P. aeruginosa might “sense” that the host on which its survival depends is subjected to an extreme degree of inflammation and vulnerability and hence represents a liability to its survival.

Given that PA-I expression was increased in response to Caco-2 cell hypoxia and normoxic recovery, a more profound decrease in TEER was expected when P. aeruginosa was apically inoculated onto Caco-2 cells exposed to these conditions. That enhanced PA-I expression in P. aeruginosa did not decrease Caco-2 cell TEER during hypoxia could be explained by the enhancing effect of hypoxia itself on Caco-2 cell barrier function. This possibility is supported by the finding that hypoxic media transferred to normoxic Caco-2 cells enhanced their resistance to P. aeruginosa (FIG. 26). This position is further supported by the finding that hypoxic Caco-2 cells resist the barrier-dysregulating property of purified PA-I, again consistent with the observation that hypoxia enhanced epithelial barrier function to the barrier-dysregulating effects of the PA-I protein of P. aeruginosa. These findings are also in agreement with the known enhancing effect of hypoxia on intestinal epithelial barrier function. Furuta et al., J Exp Med 193: 1027-1034, 2001 have demonstrated that exposure of Caco-2 cells to hypoxia increases the expression of both mucin and trefoil peptides, and they have also observed TEER to be preserved or even increased in Caco-2 cells during hypoxia. This response makes physiological sense given that under such circumstances, the intestinal epithelial surface will be vulnerable to a potentially hostile flora. However, during reperfusion, herein termed normoxic recovery, Caco-2 cells eventually succumb to the potent barrier-dysregulating effect of P. aeruginosa. This is consistent with both clinical and animal studies where the greatest alteration in intestinal permeability and systemic proinflammatory activation occurs during the reperfusion phase following ischemic injury to the intestine.

In summary, P. aeruginosa is capable of sensing and responding to local elements of host-cell stress. Host-derived bacterial signaling compounds appear to be released by intestinal epithelial cells in response to hypoxia and normoxic recovery, which are often present during critical illness and its treatment.

### Example 22

### Virulence Induction

Studies disclosed herein have been focused on the interaction between the human opportunistic pathogen Psendomonas aeruginosa and the intestinal epithelium, and more particularly, in the factors involved in causing P. aeruginosa to activate its virulence against the intestinal epithelium during host stress. Experiments were designed to test the position that following stress, soluble factors are released by the host that directly signal Pseudomonas in a manner that activates the virulence of P. aeruginosa through mechanisms that involve the quorum sensing signaling system. Within the quorum sensing regulon, it has been shown that the expression of PA-I, a lectin adhesin, can open up tight junctions leading to exotoxin A and elastase permeation across the cell barriers causing lethal sepsis.

The PA-I lectin has been shown to play a key role in the lethal effect of Pseudomonas against the intestinal epithelium. Mutant strains that lack PA-I, despite making abundant exotoxin A, have an attenuated effect on intestinal epithelial barrier function, and are completely apathogenic in a mouse model of lethal gut-derived sepsis. This is in dramatic contrast to the parental strain, which is both highly dysregulating against the intestinal epithelium and 100% lethal.

Various experiments were designed to determine whether the intestinal tract of a stressed host is a unique environment in which the virulence of P. aeruginosa is enhanced in vivo. In order to further investigate this question, a reporter strain of P. aeruginosa with GFP inserted downstream of the PA-I gene and the quorum sensing and RpoS promoters was constructed. These promoters increase PA-I expression in response to bacterial signaling pheromones, and the GFP reporter was highly responsive to these signals.

This reporter construct, was used in studies that showed that PA-I is expressed in vivo in the intestinal lumen of mice subjected to 30% surgical hepatectomy by injecting bacteria directly into the cecum and retrieving strains 24 hours later. In addition, PA-I was expressed within the lumen of mouse jejunum following segmental ischemia/reperfusion injury after injecting and retraining strains in an intestinal luminal perfusion model. Because the blood from animals in both models failed to activate PA-I expression, it was expected that the host-derived bacterial signaling factor was likely to be released locally by the intestinal epithelium itself.

The GFP PA-I reporter strains were exposed to filtered media from hypoxic cells, and were able to show a time-dependent increase in PA-I expression. These results were confirmed by northern blot.

To further understand how surgical stress and intestinal hypoxia might play a role in activating the virulence of P. aeruginosa, an experiment was designed to determine whether HTF-1-a might play a central role in this response. It is well known that hypoxia results in the accumulation of HlF-1-α in intestinal epithelial cells.

Given the increasingly important role of HIF-1-α activation in intestinal epithelial homeostasis, the question became whether HIF-1-α activation mediated the release of soluble compounds that activated P. aeruginosa virulence, as judged by expression of the PA-I lectin/adhesin.

To address this issue, an established Caco 2 cell line that has been stably transfected with HIF-1-α and its parental cell line were used. Briefly, both cell lines were grown to confluence. The media was collected and filtered through 0.22u filters to remove any potential cellular components. Media was then added to microtiter wells containing a fixed bacterial cell population of the GFP/PA-I reporter strain described above. Fluorescence was dynamically tracked over time.

Results demonstrate that there is a time-dependent induction of PA-I expression observed in GFP/PA-I reporter strains exposed to HIF-1α media compared to control. This finding was confirmed by Western blot analysis in reiterative experiments, HIF-1α activation was also confirmed by Western blot analysis.

In order to identify the potential compounds that activate PA-I, the media from three groups of Caco-2 cells were examined, namely, control cells, Caco2 cells exposed to hypoxia, and Caco2 cells with forced expression of HIF-1α. Media fractions were separated into 4 molecular weight fractions, which were added to the microtiter plates containing the PA-I/GFP reporter strains and evaluated by dynamic fluorimetry.

Results from these experiments demonstrated that media fractions with MW of <3 kDa induced PA-I expression significantly.

Further studies were performed to show that HIF and hypoxic conditions have similar effects. Because of the MW of the potential inducing compound, known genes that 1 are expressed in response to HIF-1-α activation were examined. Within this MW range, potential candidate compounds related to nucleotide metabolism were identified. In particular, adenosine showed promise as a candidate since it had been shown to be released in high concentrations following intestinal epithelial hypoxia and HIF-1-α activation.

Adenosine accumulated in the media of intestinal epithelial cells exposed to hypoxia and/or HIF-1α activation, through a mechanism that involves upregulation of 5″-nucleosidase (CD73) activity.

Reiterative experiments were therefore performed and media fractions were examined by HPLC/MS/MS for adenosine. Adenosine was greatly elevated in HIF-1-α-activated and hypoxic cell media.

The effect of adenosine on PA-I expression in the above-described reporter strain showed that the PA-I expression that was increased in the presence of adenosine was both dose- and time-dependent. Results were confirmed by Western blot. For completeness, the effects of ATP, ADP, and AMP at similar concentrations were tested and revealed no evident inducing effects.

In order to determine if adenosine was the putative component within the media of HIF-1-α activated Caco-2 cells that induced the expression of PA-I, adenosine deaminase was added to deplete the media of adenosine. Surprisingly, these experiments resulted in an even greater increase in PA-I expression, raising the possibility that a metabolite of adenosine, namely inosine, played a role in PA-I expression. Adenosine deaminase is predicted to be present in P. aeruginosa based on its DNA sequence.

Reiterative experiments were consequently performed in the presence of varying concentrations of inosine. Inosine induced PA-I expression at a concentration 10-fold less than adenosine.

Reiterative experiments to directly compare the change in PA-I expression over time between inosine and adenosine demonstrated that not only is the effect of inosine greater, but it occurs at an earlier time point. Data showed that inosine induced PA-I expression at an earlier time point and at lower cell densities (OD) compared to adenosine.

In conclusion, the present Example demonstrates that hypoxia or forced expression of HIF-1-α in Caco-2 cells resulted in the extracellular release of soluble compounds that activate the virulence circuitry of P. aeruginosa. Further, the data presented herein show that adenosine and inosine may play an important role in this response. Without wishing to be bound by theory, it appears that hypoxia and P. aeruginosa conspire to activate a virulent phenotype against the intestinal epithelium via HIF-1-α activation.

### Example 23

### HMW PEG and Acute Respiratory Distress Syndrome

ARDS is characterized by sudden, life-threatening lung failure with diffuse alveolar infiltrate, reduced arterial oxygenation, and pulmonary edema. Acute lung injury, ALI, is the end result of common pathways initiated by a variety of local or systemic insults. HMW PEG compounds are inert and non-toxic polymers that act as a surrogate mucin lining providing protection against bacterial infections on intestinal epithelial cells. HMW PEG was expected to provide protective effects on lung endothelium by attenuating endothelial cell (EC) activation that results in barrier dysfunction. The work described herein examines the effects of a high molecular weight HMW PEG (HMW PEG 15-20 kDa) on cultured human pulmonary microvessel EC exposed to barrier disruptive agents. EC were cultured on microelectrodes and changes in transendothelial electrical resistances (TEER) were measured to assess alterations in paracellular permeability. HMW PEG induced a rapid, dose-dependent increase in TEER similar to barrier-enhancing lipid such as sphingosine 1-phosphate (S1P). Optimal concentration of 7.5-9% HMW PEG induced a robust increase in TEER from 2500 to 5000 ohm which was sustained for 40 hours. In comparison to S1P which induced a 40% increases resistance in 10-15 minutes, the maximum barrier enhancing effect of HMW PEG was achieved in about 45 minutes, but with 100% increase in resistance. With a 2-fold increase in resistance, HMW PEG is the most-potent barrier enhancing agent tested among all the barrier-enhancing agents such as S1P, FTY720, phospho-FTY720, and HGF. Immunofluorescence data revealed that HMW PEG altered the EC actin cytoskeleton to form a defined cortical actin ring that may help strengthen cell-cell junctional adhesion. HMW PEG rapidly induced dephosphorylation of ERK and MLC as early as 1 minute and completely inhibited thrombin-induced ERK and MLC phosphorylation. More importantly, pretreatment with HMW PEG for 1 hour attenuated thrombin-induced endothelial barrier dysfunction. In summary, HMW PEG activates a rapid, actin-associated, barrier-enhancing signal transduction pathway in EC, which may have therapeutic potential to prevent and reverse pulmonary edema.

High Molecular Weight PolyEthylene Glycol (HMW PEG; P-2263, MW 15,000-20,000 kDa, Lot# 042K0199 and 045K0103), sphingosine 1-phosphate, human thrombin (cell culture grade), fetal bovine serum (FBS), phosphate buffer saline, telostein gelatin, and bovine serum albumin were purchased from Sigma-Aldrich (St. Louis, Mo.). All primary antibodies were generated against human antigens in which anti-ERK, anti-phospho-ERK (Thr202/Tyr204), anti-MLC, and anti-diphospho-MLC (Thr18/Ser19) were from Cell Signaling Technology (Beverly, Mass.). Anti-mouse and anti-rabbit secondary antibodies conjugated to horse radish peroxidase, enhanced chemiluminescence (ECL), and ECL-Plus were purchased from Amersham Biosciences, Inc /GE Health Sciences (Piscataway, N.J.). Texas Red-phalloidin, anti-mouse and rabbit Alexa 488 secondary antibodies, and Prolong mounting solution were from Molecular Probes (Eugene, Oreg.).

Human lung microvessel endothelial cells (HLMVEC) were purchased from Clonetics/BioWhittaker, Inc. (Walkersville, Md.) and grown in the manufacturer's recommended Endothelial Growth Medium-2-Microvessel consisting of defined growth factors and supplemented with additional FBS up to 10% final concentration. Cells were grown at 37° C. in a 5% CO₂ incubator and used from passages 6-9. For experiments, ECs were plated at an appropriate density (875,000 cells/D60; 300,000 cells/D35; 100,000 cells/CultureSlide; 75.000 cells/ECIS well) and used 3 days after plating, unless otherwise noted. Media were changed 1 day prior to experimentation.

Small interfering RNA (siRNA) targeting Racl was obtained as a pool of four siRNA duplexes from Dharmacon (Lafayette, Colo.). Control siRNA (siCONTROL) targeting non-human protein, luciferase, was used to minimize off-target silencing. The silencing protocol was optimized to allow transfection of cells plated at high density and on non-conventional substrates such as gold electrodes. ECs were plated accordingly and treated with siRNA 3 hours later using one half volume typical for a dish/well. SiRNA was premixed with transfection reagent for 20 minutes and then diluted with serum medium for a final concentration of 25-100 nM siRNA and 6 μl/ml siPORT Amine (Ambion, Austin, Tex.). After 24 hours, an equal volume of serum medium was added to the cells containing siRNA. Silenced cells were used 3-5 days post-transfection, and the medium was replaced a day prior to all experiments.

For immunofluorescence studies, ECs were seeded into 8 chamber, collagen-coated, Culture Slides (BD Biosciences, Lexington, Ky.). After agonist stimulation, cells were washed with phosphate-buffered saline (PBS) once and fixed with 3.7%) formaldehyde for 5 minutes, permeabilized with 0.25% Triton X-100 in PBS for 3 minutes, washed and probed with primary antibodies at 1:100-1:200 dilution for 45 minutes. F-actin was probed with Texas Red-phalloidin at a 1:200 dilution. Secondary antibodies were diluted 1:200 and incubated for 30 minutes. Slides were mounted with Prolong™ anti-fade reagent. Stained cells were visualized using a Nikon Eclipse TE2000 inverted microscope (Nikon Inc., Melville, N.Y.).

An electrical cell-substrate impedance sensing (ECIS) system (Applied Biophysics, Troy, N.Y.) was used to measure transendothelial electrical resistance (TEER) with EC's grown on gold microelectrodes (Tiruppathi, C 1992a b). ECs were plated directly onto gold microelectrodes of an ECIS plate (8W10E) and cultured for 2-3 days. Confluency was assessed as minimum basal resistance of 2000 ohms for HLMVEC, Data pooling and analyses were performed using Epool software created by Kane L. Shaphorst, M.D.

Western blots were prepared as follows. After agonist stimulation, cells were washed with cold Endothelial Basal Medium (EBM) once and extracted with 0.3% SDS lysis buffer (300 μl/D60) containing protease inhibitors (1 mM EDTA, 1 mM PMSF, 1 mM sodium orthovanadate, 1 mM sodium fluoride, 0.2 TIU (trypsin inhibitor umt)/ml aprotinin, 10 μM leupeptin, 5 μM pepstatin A). DNA was sheared with a 26-gauge syringe. Each sample was boiled for 5 minutes, and diluted with 5× sample buffer (0.56 M Tris pH 7.0, 10% SDS, 25% β-ME, 25% sucrose, 0.025% bromophenol blue). Sample proteins were separated with a 10% or 15% homogeneous SDS-PAGE gel using a Mini-Protean II apparatus (Bio-Rad, Hercules, Calif.). Proteins were transferred onto Immobilon-P PVDF membrane (Millipore, Bedford, Mass.), and immunoblotted with primary antibodies (1:1000, 4° C., overnight) followed by secondary antibodies conjugated to HRP (1:5000, room temperature, 30 minutes) and detected with enhanced chemiluminescence (Pierce ECL or SuperSignal West Dura, Pierce Biotechnology, Rockford, Ill.) on Biomax MR film (Kodak, Rochester, N.Y.).

For experiments measuring calcium, cells were grown on 20×40 mm coverslips and calcium flux measurements were performed using conventional techniques.

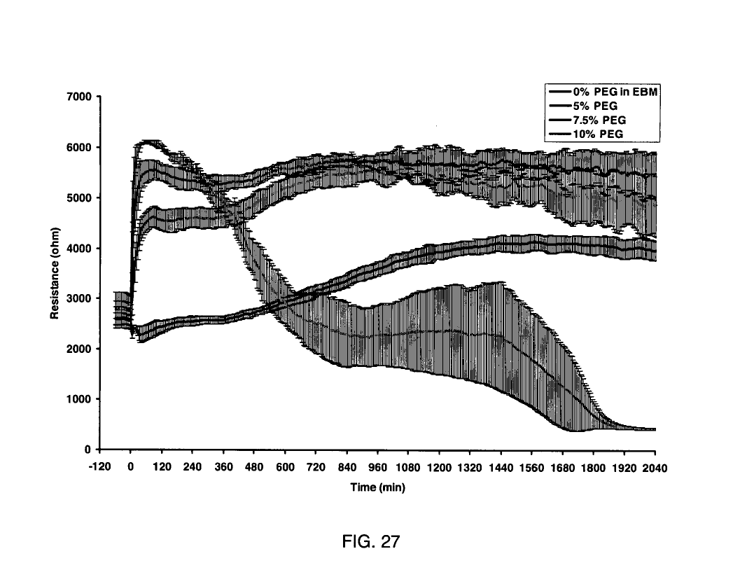

The results of the experiments described in this Example established that HMW PEG induced a potent and sustained endothelial barrier enhancement. Agents that mediate barrier protection in endothelial cells can act by increasing the tightness of junctions, resulting in enhanced barrier function, or by preventing agonist-induced changes in barrier permeability without altering basal barrier function. It was expected that HMW PEG could be used as a therapeutic agent to block or reverse agonist-induced endothelial barrier dysfunction, and this expectation was confirmed by examining the effect of HMW PEG alone on endothelial barrier permeability. To measure changes in barrier permeability. HMW PEG was added to endothelial cells grown on gold microelectrodes and changes in resistances were measured over time, with increases in resistance corresponding to enhanced barrier function. Since HMW PEG is relatively insoluble, concentrated stocks of HMW PEG could not be made for use as a low-volume additive to the media of cells being tested. Rather, various final concentrations of HMW PEG were achieved by dissolving HMW PEG quantities in Endothelial Basal Medium (EBM), warmed to 37° C., and used a s replacement for serum-containing endothelial growth medium (EGM). FIG. 27 demonstrated that HMW PEG induced a dose-dependent increase in endothelial barrier function as measured by increased resistance (up to 6100 ohm) from a basal resistance of approximately 2500 ohm. In comparison to other known barrier-enhancing agents that induce maximal resistance up to 3800 ohm, HMW PEG appears to be the most potent barrier-enhancing agent examined thus far. Furthermore, both 5 and 7.5% HMW PEG induced a sustained barrier enhancement that lasted for more than 40 hours. However, higher HMW PEG concentrations elicited a barrier enhancement that was not sustained, dropping below baseline by 10 hours post-treatment. The viscosity of 10% HMW PEG was extremely high such that it may have interfered with nutrient/gas exchange, resulting in toxicity. Nevertheless, the optimal HMW PEG concentration dissolved in EBM appears to have been approximately 7.5% HMW PEG, which conferred protection with minimal toxicity.

Since the viscosity of HMW PEG increased as concentration increased, the effect of HMW PEG alone on electrical resistance measurements (in the absence of cells; see Table 3) was examined. Highly viscous 10% HMW PEG increased resistance measurements by only 81 ohm in comparison the 3842 ohm difference when HMW PEG was added to cells. Therefore, HMW PEG alone did not significantly contribute to the increased resistance; rather, the cells were responding to HMW PEG by tightening the junctional adhesion of cells, resulting in enhanced barrier function. Note that this conclusion is also evident by the prolonged 10% HMW PEG treatment in which the resistance ultimately dropped to a level near 500 ohms (FIG. 27).

Table 3. Resistance measurements induced by HMW PEG on electrodes without cells in comparison to electrodes with HLMVEC. To verify that HMW PEG did not produce an artifactual effect in the ECIS assay, measurements of various concentrations of HMW PEG with and without cells were taken. The difference of resistance measurements of 10% HMW PEG relative to 0% was calculated with and without cells to demonstrate the insignificant effect of HMW PEG per se on electrical resistance readings. Thus, changes in resistances observed in experiments with cells were a result of cellular responses to HMW PEG.

TABLE 3

| | Unnormalized Data | | | | | Normalized Data | | | |
|---|---|---|---|---|---|---|---|---|---|
| PEG | 0.0% | 2.5% | 5.0% | 10.0% | difference (10 − 0%) | 0.0% | 2.5% | 5.0% | 10.0% |
| resistance (ohm) without cells | 442.38 | 457.63 | 475.75 | 523.25 | 80.88 | 1.00 | 1.03 | 1.08 | 1.19 |
| resistance (ohm) with HLMVEC | 2344.65 | 3927.45 | 4788.25 | 6187.05 | 3842.40 | 1.00 | 1.68 | 2.04 | 2.64 |

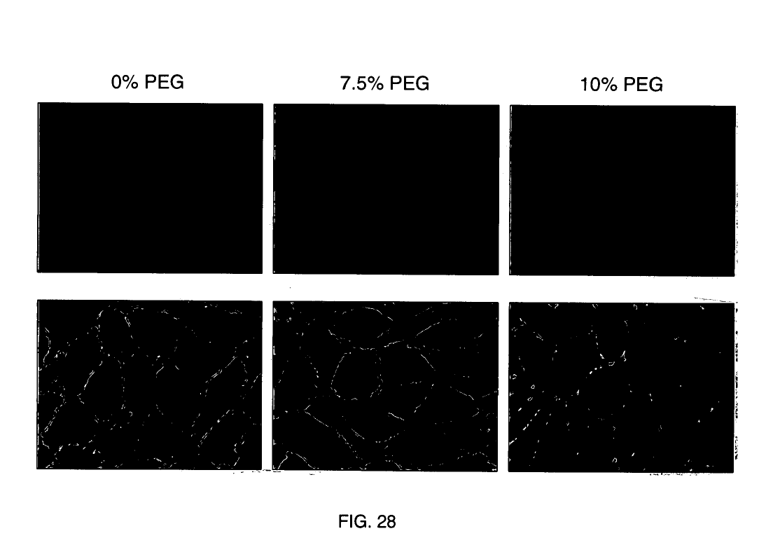

The mechanism(s) of the rapid and potent barrier enhancement observed during the early phase of cell exposure to HMW PEG was investigated, and it was found that HMW PEG induced cortical actin formation along with increased VE-cadherin at cell junctions. HLMVEC were stimulated for 1 hour with 7.5% HMW PEG, 10% HMW PEG, or left unstimulated, and then fixed and stained with probes for VE-cadherin and filamentous actin (FIG. 28). VE-cadherin is a key junctional adhesion protein in endothelial cells, and actin cytoskeleton is a key structure involved in cellular motility that has been implicated in endothelial barrier enhancement. Addition of 7.5% HMW PEG increased cortical actin formation and 10% HMW PEG caused a further increase, along with actin bundling, as observed by the highly delineated and thinner cortical actin ring. Increase in cortical actin formation has also been observed with other barrier-enhancing agents, such as S1P, HGF, and simvastatin. Similarly, addition of 7.5% HMW PEG increased VE-cadherin staining at the junctions, correlating with enhanced barrier function. At higher HMW PEG concentration, VE-cadherin was still present in cell-cell junctions, but absent in some multi-cellular contacts. The presence of VE-cadherin is well-known to be associated with increased junctional adhesion, and the absence of VE-cadherin is associated with junctional disruption. With only a 1-hour treatment of 10% HMW PEG, junctional adhesion appeared to begin decreasing (FIG. 28), consistent with the eventual hairier dysfunction seen with prolonged treatment of cells with 10% HMW PEG (FIG. 27). Nevertheless, HMW PEG induced actin and VE-cadherin reorganization, which correlated with other barrier-enhancing models.

Activation of a signal transduction pathway can be revealed by an increase in target protein phosphorylation, and the data show that HMW PEG downregulated ERK and MLC phosphorylation. ERK and MLC phosphorylation have been associated with treatment with barrier-disrupting agents such as thrombin. However, recent evidence has shown that barrier-enhancing agents may also increase ERK and MLC phosphorylation in a spatially specific region of the cell that promotes contrasting effectors. Specifically, thrombin induces the MLC phosphorylation of central stress fibers, which promotes contraction and barrier dysfunction, as opposed to S1P, which induces MLC phosphorylation at the cortical region to stabilize cell-cell junctions and promote barrier enhancement. Thus, it has been shown that barrier-enhancing S1P activated signal transduction pathways that potently increases ERK and MLC phosphorylation (Garcia et al., JCI 2001). Since HMW PEG similarly induced a rapid barrier enhancement along with increased cortical actin formation, the capacity of HMW PEG to activate similar pathways was examined.

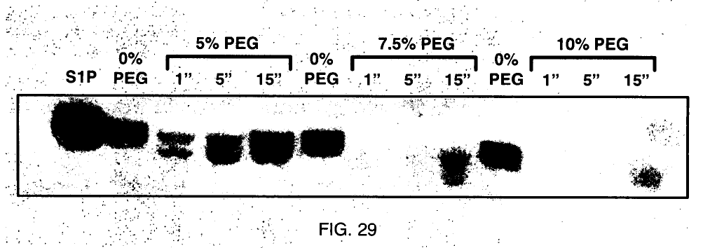

Cells were treated with 0, 5, 7.5, or 10% HMW PEG for various times (1,5, and 15 minutes). Positive controls for ERK and MLC activation were included using cells treated with 1 μM S1P for 5 minutes. Lysates were collected and processed for electrophoretic separation and Western blot probing using phospho-specific antibodies to ERK and MLC (FIG. 29). Rather than a drastic increase in ERK phosphorylation as seen with S1P, both 7.5% and 10% HMW PEG completely blocked basal ERK phosphorylation, indicating a divergence in the signaling pathways for S1P and HMW PEG. Furthermore, 5% HMW PEG, which enhanced barrier resistance, decreased basal ERK phosphorylation by as early as 1 minute post-delivery, but the inhibitory effect was diminished by 15 minutes post-delivery. Similar results on MLC phosphorylation were observed with lower MLC phosphorylation. In summary, HMW PEG does not activate similar signal transduction pathways as S1P, which indicates alternate signaling pathways.

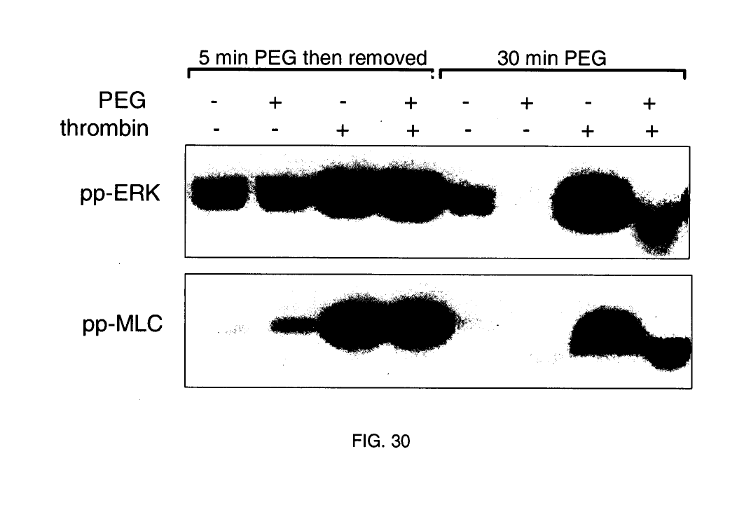

The ability to detect thrombin-induced ERK and MLC phosphorylations was used to develop an assay to determine whether HMW PEG could affect thrombin's signaling pathway. Since HMW PEG has an inhibitory effect on basal ERK and MLC phosphorylation, the ability of HMW PEG pretreatment to inhibit a barrier-disrupting signaling pathway, as detected by ERK and MLC phosphorylation (FIG. 30), was examined. Cells were pretreated with 7.5% HMW PEG for 30 minutes and then stimulated with vehicle or thrombin (1U/ml, 15 minutes). Cell lysates were processed for Western blot probing with phospho-specific antibodies for ERK and MLC. Thrombin induced a potent ERK phosphorylation that was completely blocked by HMW PEG pretreatment. Furthermore, HMW PEG significantly attenuated thrombin-induced MLC phosphorylation, presumably localized in the central stress fibers. Previous data on epithelial cells indicated that HMW PEG induced its effect rapidly (as early as 1 minute post-delivery) and adhered to cell surface despite removal efforts or washes. To determine if the presence of HMW PEG in the endothelial cell model is necessary to inhibit thrombin-induced ERK and MLC phosphorylation, cells were pretreated with 7.5% HMW PEG for 5 minutes followed by removal with EBM replacement (FIG. 30). After 25 minutes incubation in HMW PEG-free medium, cell lysates were collected and processed for Western blots. Surprisingly, the ability of HMW PEG to inhibit thrombin-induced ERK and MLC phosphorylation were abolished. Also, removal of HMW PEG caused barrier function to return to its basal level. When HMW PEG was added to EC and not removed, HMW PEG potently blocked and attenuated thrombin-induced ERK and MLC phosphorylations, demonstrating that HMW PEG inhibited thrombin-induced barrier dysfunction.

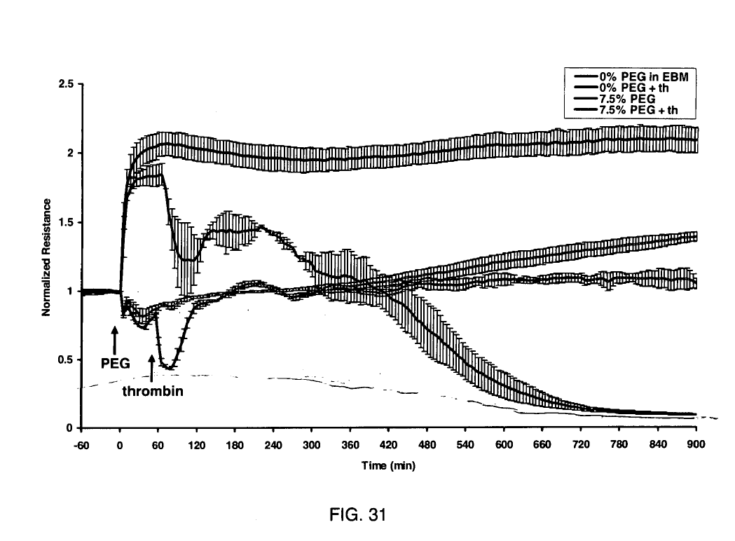

Since HMW PEG blocked and attenuated thrombin-induced ERK and MLC signaling pathways, an experiment was performed to determine whether HMW PEG could block thrombin-induced barrier dysfunction, as measured by changes in electrical resistance. Cells grown on microelectrodes were pretreated with 7.5% HMW PEG reconstituted in warm EBM and added to the cells at time=0 minutes, in comparison to control exposed only to EBM. After 1 hour of HMW PEG pretreatment in which the curves plateaued (maximum resistance was reached and maintained; FIG. 31), thrombin (1 U/ml) was added to selected wells. In control cells, thrombin induced a decrease in resistance (over 50%) that recovered in approximately 1 hour (FIG. 31). The maximum decrease in resistance of approximately 1200 ohm represented barrier disruption characterized by cell-cell junctional gap formations. Addition of HMW PEG immediately enhanced basal resistance 2-fold, which was sustained for more than 40 hours. Subsequent challenge of HMW PEG-pretreated cells with thrombin induced a characteristic decrease in resistance that did not fall below baseline or basal resistance levels, indicating protection from barrier disruption. However, the recovery phase unexpectedly dropped below baseline 5 hours post-thrombin challenge. By 12 hours of thrombin exposure, the resistance dropped to approximately 500 ohm, consistent with complete barrier disruption and possible cell toxicity. Neither 7.5% HMW PEG nor thrombin alone caused the resistance to drop completely. Because thrombin caused a dynamic and short-term barrier disruption, it is interesting to observe that there was a significant decrease in resistance at more than 600 minutes, relative to the basal control. The sensitivity of the cells to external factors appeared to be increased, possibly due to being in serum-free medium. Cells were routinely incubated in serum-free medium to induce quiescence and to eliminate variables in the serum, such as growth factors, that would interfere with short-term studies. However, depletion of serum and growth media for extended periods of time has been shown to be detrimental to lung endothelial cells. Therefore, the effect of HMW PEG reconstituted in serum medium containing growth factors and 10% FBS was examined.

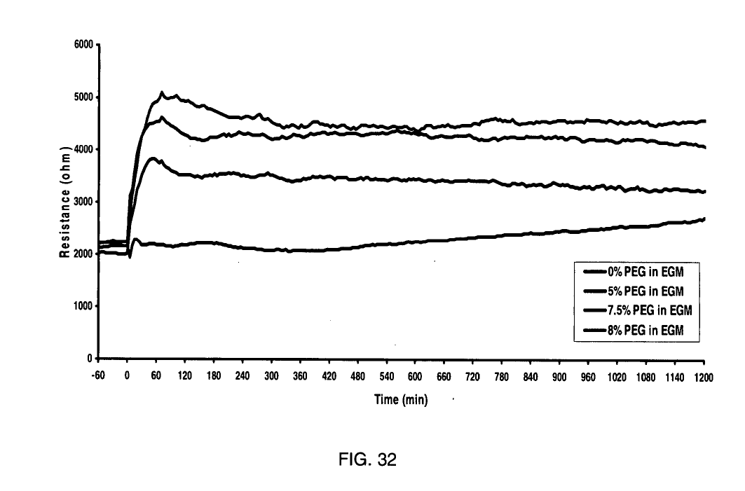

In view of the potential negative effects of serum-free conditions (EBM), HMW PEG was reconstituted in serum medium or endothelial growth medium (EGM) and dose-response relationships were determined using ECIS (FIG. 32). HMW PEG in EGM induced a dose-dependent increase in barrier enhancement similar to HMW PEG in EBM, but with a slightly lower maximum. For example, the maximum increase in resistance for 7.5% HMW PEG in EGM is 4500 ohm compared to 5500 ohm for HMW PEG in EBM. Higher concentrations of HMW PEG in EGM still caused barrier disruptions similar to HMW PEG in EBM. The results for 9% and 10% HMW PEG in EGM were not significant due to large error bars, and it is preferred that HMW PEG solutions of less than 9% be used in the methods according to the invention. Consistently, 8% HMW PEG in EGM yielded barrier enhancements near 5000 ohm, and thus was used for subsequent experiments.

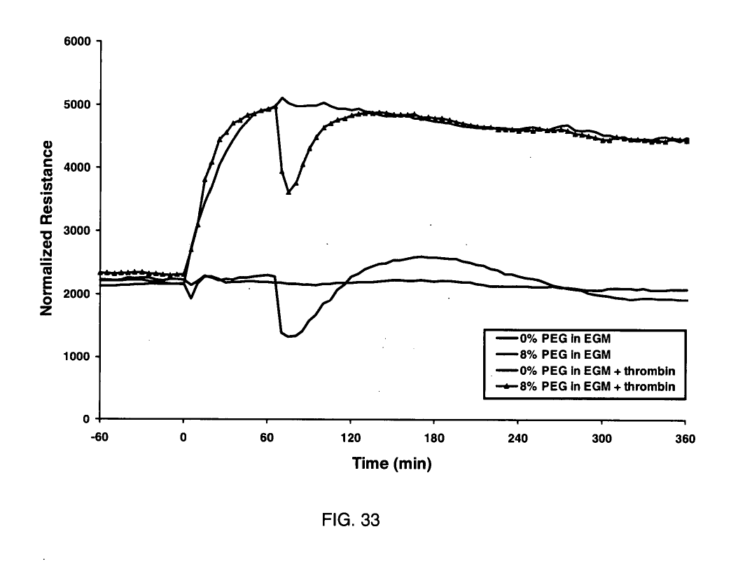

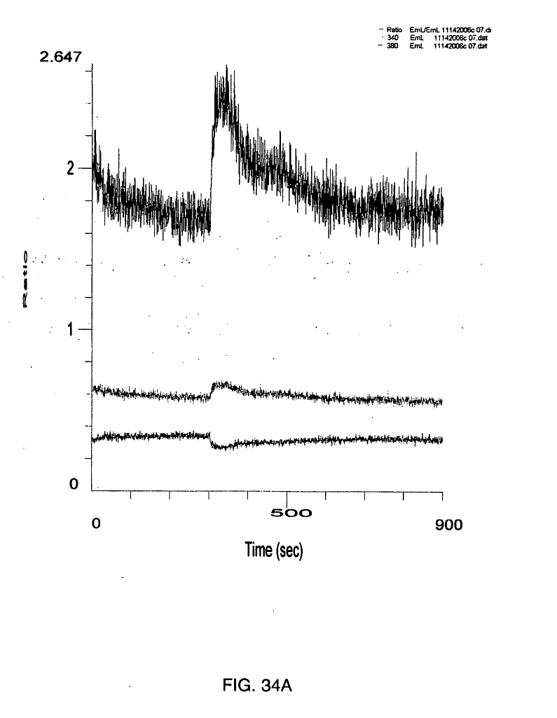

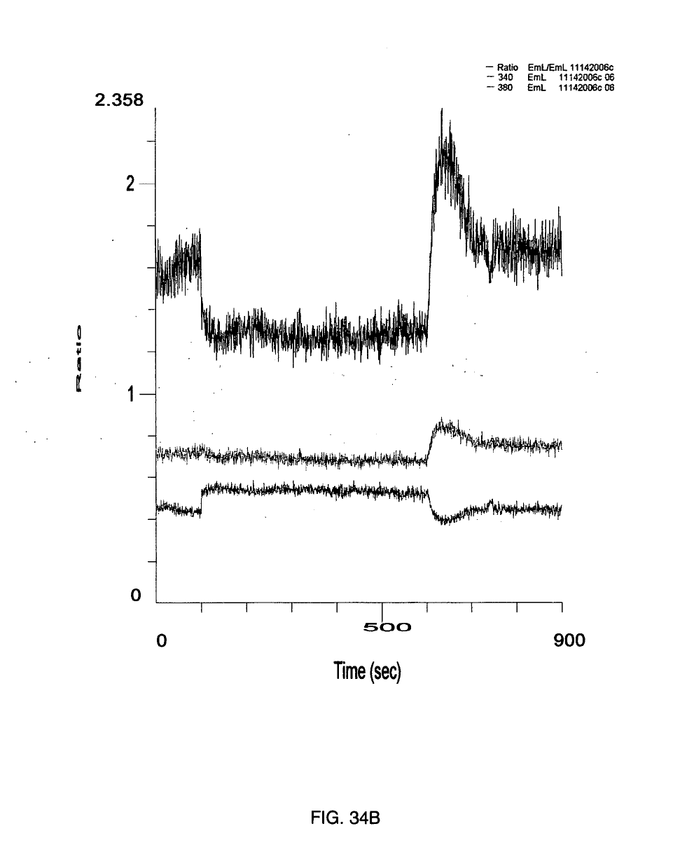

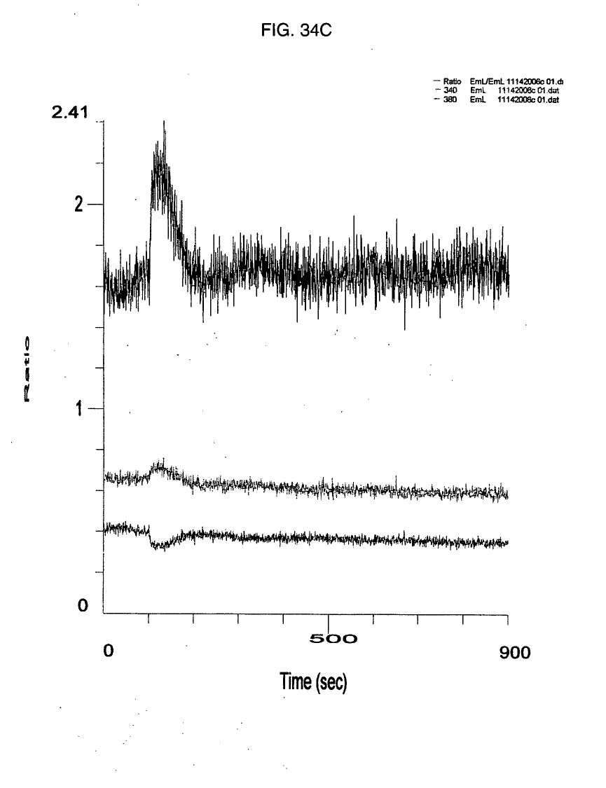

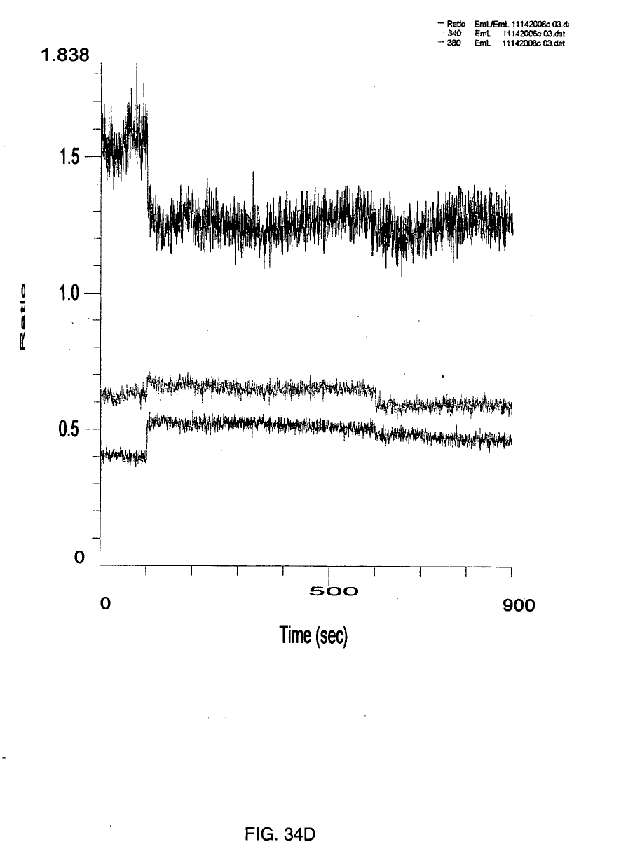

Cells grown in EGM were pretreated with control EGM or with 8% HMW PEG reconstituted in EGM at time= 0 minutes. After a 1-hour incubation, cells were challenged with thrombin (1 U/ml). In untreated cells, thrombin induced a characteristic transient barrier disruption, whereas pretreatment with HMW PEG enhanced barrier resistance (FIG. 33). Thrombin challenge of HMW PEG-pretreated cells resulted in a decrease in resistance with a minimum of 3500 ohm, well above the basal resistance of 2200 ohm. Furthermore, restoration of barrier resistance to the 5000-ohm level occurred within 60 minutes post-thrombin challenge, indicating that HMW PEG in serum-containing medium protected barrier function by maintaining heightened barrier resistance significantly above basal levels. Related to these findings is the discovery that phorbol ester pretreatment of cells attenuates the barrier-sustaining effect of HMW PEG. Consistently, studies on calcium flux showed that HMW PEG induced a decrease in intracellular calcium concentration that blocked an S1P-induced, but not a thrombin-induced, spike in intracellular calcium concentration (FIG. 34).

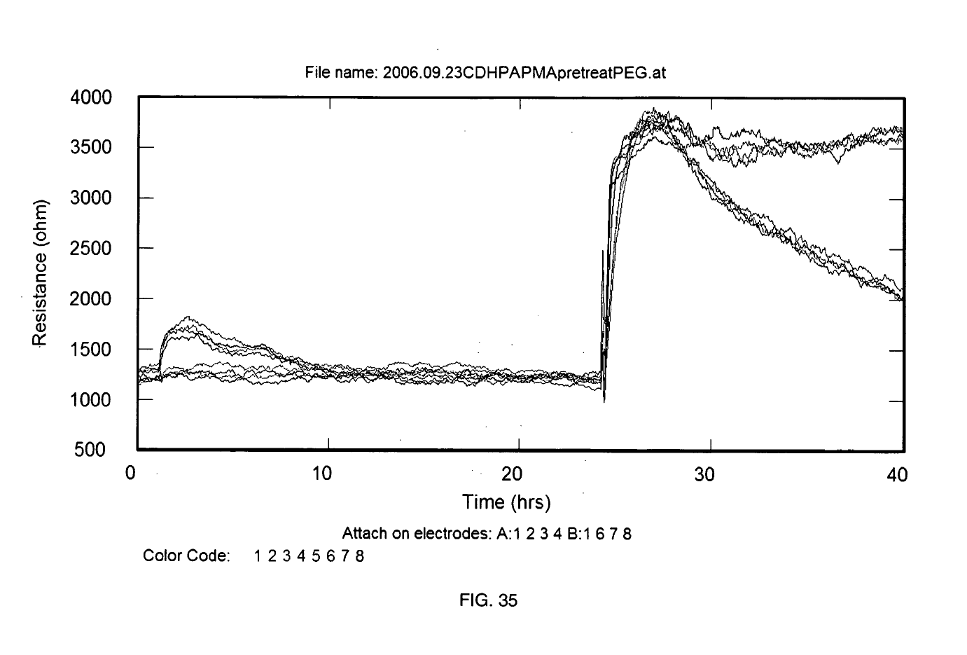

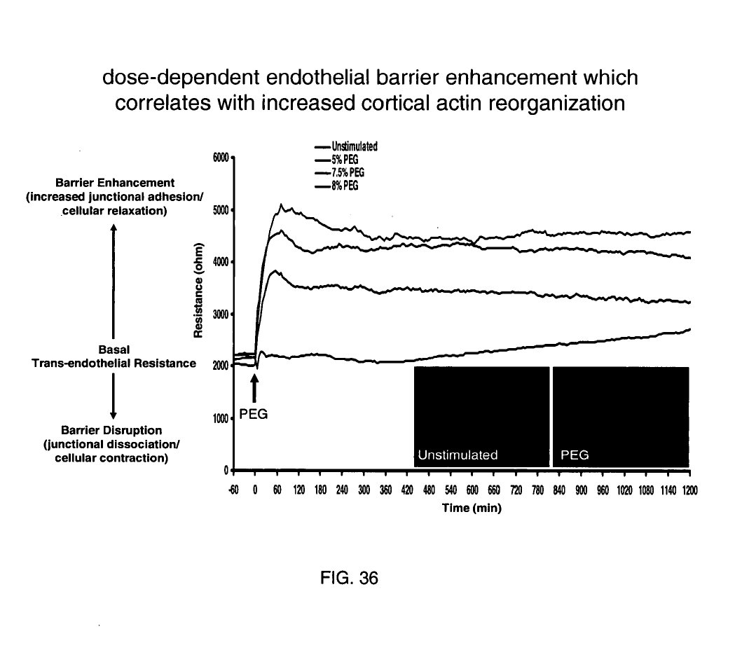

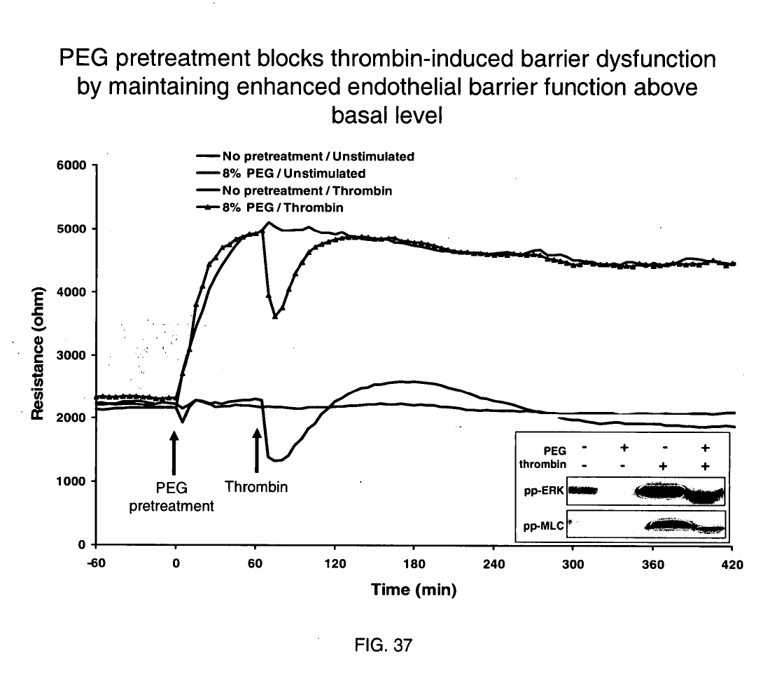

In another experiment, cells pretreated with phorbol ester were shown to exhibit an increased rate of recovery from a spike in TEER attributable to exposure to HMW PEG 24 hours after phorbol ester exposure (FIG. 35). The data showed that phorbol ester pretreatment attenuated the barrier-sustaining effects of HMW PEG, which is consistent with phorbol ester inducing a downregulation of key signaling proteins involved in maintaining endothelial barrier function upon HMW PEG challenge. This led to an additional experiment more generally exploring the pretreatment of endothelial cells. Endothelial cells were pretreated with any one of the following: 1 μM S1P (24 hours), 10 μM FTY720 (24 hours), 10 μM FTY analog (24 hours), 100 nM PMA (24 hours), 100 ng/ml PTX (4 hours), or 10 μM ionomycin (4 hours). Subsequently, pretreated EC samples were challenged with either 1 μM S1P or 8% HMW PEG. The results are presented in Table 4.

TABLE 4

| | | | FTY | | | |
|---|---|---|---|---|---|---|
| | S1P | FTY720 | analog | PTX | Ionomycin | PMA |
| S1P | No effect | Blunted | Blunted | Blocked | N/A | Blocked |
| stimulation | | | | | | |
| HMW | No effect | No effect | No effect | No/marginal | Blocked | Attenuate |
| PEG | | | | effect | | HMW |
| stimulation | | | | | | PEG |
| | | | | | | barrier- |
| | | | | | | sustaining |
| | | | | | | effect |

In summary, HMW PEG is one of the most potent barrier-enhancing agents to be tested on human lung microvessel endothelial cells. An exemplary concentration of HMW PEG that is therapeutically effective is 8% HMW PEG, the in vitro dose demonstrated to enhance lung endothelial barrier function, as well as to protect EC from thrombin-induced barrier disruption. HMW-PEG exposure also leads to morphological changes of cortical actin formation and VE-cadherin recruitment to the junctions, in addition to the ability of HMW PEG to inhibit ERK and MLC signaling pathways. The mechanism by which HMW PEG provides effective protection against agonist-induced barrier dysfunction has also been disclosed. HMW PEG must be prepared in serum medium conditions and remain in that form during cell contact to induce a protective effect. HMW PEG in serum-free conditions resulted in less desirable, and sometimes undesirable, outcomes; removal of HMW PEG abolished these effects. The use of HMW PEG as a pretreatment against thrombin challenge demonstrated that HMW PEG provides protective benefits to cells, including endothelial cells such as lung microvessel endothelial cells.

### Example 24

### HMW PEG and Neonatal Necrotizing Colitis

A high molecular weight co-polymer, such as a high molecular weight PEG-like compound, protects organisms against neonatal necrotizing enterocolitis (NEC), sepsis, lethal toxemia, and ARDS. In neonatal necrotizing enterocolitis (NEC), bacterial invasion into the gut epithelium and toxemia play a critical role in the persistently high mortality characteristic of this disease. The HMW PEGs according to the invention were expected to be therapeutically useful in addressing NEC in accordance with the disclosures herein.

A standardized model of NEC was used. Mouse pups were randomized to receive infant formula with water or 5% HMW PEG. Pups were fed by gavage for 96 hours and NEC was determined. Intestinal segments were examined for the HMW PEG effect by atomic force microscopy (AFM). To determine if 5% HMW PEG protected against toxemia, mice were intravenously (IV) administered 5% HMW PEG followed by a lethal intraperitoneal (IP) dose of exotoxin A, a cytotoxin of P. aeruginosa. Finally, HMW PEG was tested in endothelial cells exposed to exotoxin A.

Results showed that NEC was significantly reduced in animals fed HMW PEG (68% reduction n=14/group P<0.05). AFM demonstrated that HMW PEG formed a topographically tight covering on the epithelium and protected against bacterial invasion and epithelial apoptosis. IV administration of 5% HMW PEG protected against a lethal dose of exotoxin A (75% reduction in mortality, P<0.001). HMW PEG completely protected the endothelium against exotoxin A-induced cytotoxicity. Synchrotron X-ray beam analysis demonstrated the intercalation of 5% HMW PEG into bilipid membranes with the formation of repellant polymer brushes. Mice fed 5% HMW PEG (oral) for one month displayed normal growth patterns and appeared healthy. In addition to the therapeutic and prophylactic activities of HMW PEG-like compounds according to the invention, these compounds have demonstrated no biological or clinical toxicities.

### Example 25

### HMW PEG Protection Against Thrombin-Induced Endothelial Barrier Dysfunction

This Example presents data establishing that high-molecular weight polyethylene glycol-like compounds, such as HMW PEG, protect against thrombin-induced endothelial barrier dysfunction by inducing actin cytoskeleton rearrangement, which results in robust enhancement of endothelial cell barrier integrity.

As noted elsewhere, ARDS is characterized by sudden, life-threatening lung failure with diffuse alveolar infiltrate, reduced arterial oxygenation, and pulmonary edema. HMW polyethylene glycol (PEG) compounds are generally inert and non-toxic polymers that can act as a surrogate mucin lining, providing protection against bacterial infections on intestinal epithelial cells. HMW PEG-like compounds were expected to provide similar protective effects on lung endothelium by attenuating endothelial cell (EC) activation that results in barrier dysfunction. This study reflects an examination of the effects of a high molecular weight PEG on cultured human pulmonary microvessel EC exposed to barrier disruptive agents.

Endothelial cells (EC) line the entire vasculature and provide a semi-permeable barrier between blood and tissue. Pulmonary endothelium is the largest vascular bed in the human body and dysregulation/inflammation of the vascular barrier causes alveolar flooding that may lead to multiple organ failure and mortality.

HMW PEGs, as described herein, are inert and nontoxic compounds disclosed as being useful in the treatment of intestinal epithelium infections. HMW PEGs anchor to living surfaces and exert major changes in surface electric charge, hydrophobicity, and van der Waals forces facing approaching proteins.

As disclosed herein, HMW PEGs are expected to be useful in treatments to regulate vascular barrier function. The experiments disclosed in this Example establish that HMW PEGs reverse vascular barrier dysfunction, which provides protection against edemagenic agents such as thrombin and lipopolysaccharide (LPS).

The reagents used in the experiments described in this Example included polyethylene glycol, sphingosine 1-phosphate (S1P), human thrombin (cell culture grade), fetal bovine serum (FBS), phosphate-buffered saline (PBS), telostein gelatin, and bovine serum albumin, all of which were obtained from Sigma-Aldrich. All primary antibodies were generated against human antigens; anti-ERK, anti-phospho-ERK (Thr202/Tyr204), anti-MLC, and anti-diphospho-MLC (Thr18/Ser19) antibodies were from Cell Signaling Technology. Anti-mouse and anti-rabbit secondary antibodies conjugated to horseradish peroxidase (HRP), enhanced chemiluminescence (ECL), and ECL-Plus were purchased from Amersham Biosciences, Inc /GE Health Sciences. Texas Red-phalloidin, anti-mouse and rabbit Alexa 488 secondary antibodies, and Prolong mounting solution were from Molecular Probes.

Human lung microvessel endothelial cells (HLMVEC) and human pulmonary artery endothelial cells (HPAEC) were purchased from Clonetics/BioWhittaker, Inc. and grown in the manufacturer's recommended Endothelial Growth Medium-2-Microvessel. Cells were grown in an incubator at 37° C. in 5% CCb and used between passages 6-9. For experiments, ECs were plated at appropriate densities (875,000 cells/D60; 300,000 cells/D35; 100,000 cells/CultureSlide; 75,000 cells/ECIS well) and used 3 days after plating, unless otherwise noted. Medium was changed one day prior to experimentation.

Immunofluorescence was used to detect various proteins. ECs were seeded onto 8-chamber, collagen-coated CultureSlides. After agonist stimulation, cells were washed with phosphate-buffered saline once and fixed with 3.7% formaldehyde for 5 minutes. Fixed cells were permeabilized with 0.25% Triton X-100 in PBS for 3 minutes, washed and probed with primary antibodies (1:100-1:200 dilution) for 45 minutes. F-actin was probed with Texas Red-phalloidin at 1:200 dilution. Secondary antibodies were diluted 1:200 and incubated with cells for 30 minutes. Slides were mounted with Prolong™ anti-fade reagent. Stained cells were visualized using a Nikon Eclipse TE2000 inverted microscope.

For electrical resistance measurements (transendothelial electrical resistance or TEER), an electrical cell-substrate impedance sensing (ECIS) system (Applied Biophysics) was used with ECs grown on gold microelectrodes. ECs were plated directly onto gold microelectrodes of an ECIS plate (8W10E) and cultured for 2-3 days. Data pooling and analyses were performed using Epool software created by Kane L. Shaphorst, M.D.

After agonist stimulation, cells were washed with cold Endothelial Basal Medium (EBM) once and extracted with 0.3% SDS lysis buffer (300 μl/D60) containing protease inhibitors. DNA was sheared by passage through a 26-gauge syringe. Each sample was boiled for 5 minutes and diluted with 5× sample buffer. Sample proteins were separated with a 10% or 15% homogeneous SDS-PAGE gel using the Mini-Protean II (Bio-Rad) system. Proteins were transferred onto Immobilon-P PVDF membrane, and immunoblotted with primary antibodies (1:1000, 4° C., overnight) followed by secondary antibodies conjugated to HRP (1:5000, room temperature, 30 minutes) and detected with enhanced chemiluminescence on Biomax MR film.

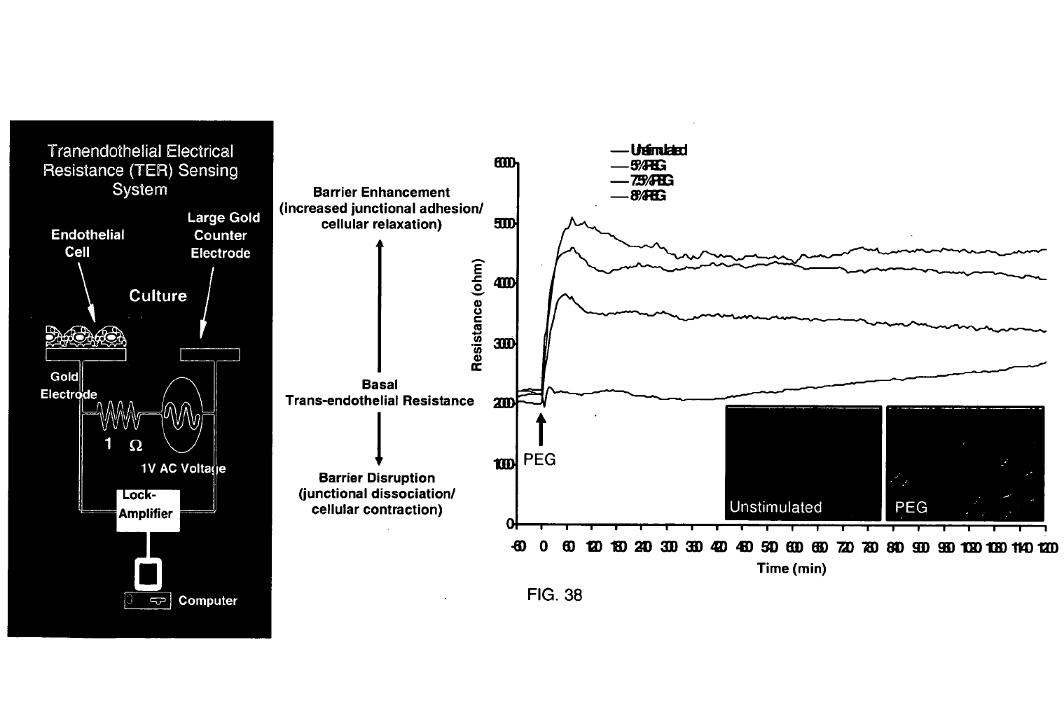

As shown in FIG. 38, PEG induced dose-dependent increases in endothelial barrier enhancement and altered F-actin distribution. HLMVEC were plated on gold microelectrodes and grown to confluency. Changes in endothelial cell barrier permeability were assessed upon addition of 5-8% HMW PEG, in which increased resistance corresponded to enhanced barrier integrity. The inset in FIG. 38 shows immunofluorescence images of unstimulated vs. HMW PEG-stimulated cells stained for F-actin. HMW PEG exposure (10%, 1 hour) resulted in increased cortical F-actin formation and bundling. In addition, VE-cadherin is recruited to the junctions.

Resistance measurements induced by HMW PEG on electrodes without cells were compared to resistance measurements induced by HMW PEG on electrodes with HLMVEC, and the data is provided in Table 3. To verify that HMW PEG did not have an artifactual effect in the EOS assay, measurements of various concentrations of HMW PEG, with and without cells, were taken. The differences in resistance measurements of 10% PEG relative to 0% was calculated with and without cells to demonstrate the insignificant effect of EIMW PEG on electrical resistance readings. Thus, changes in resistances observed in experiments with cells were a result of cellular responses to HMW PEG.

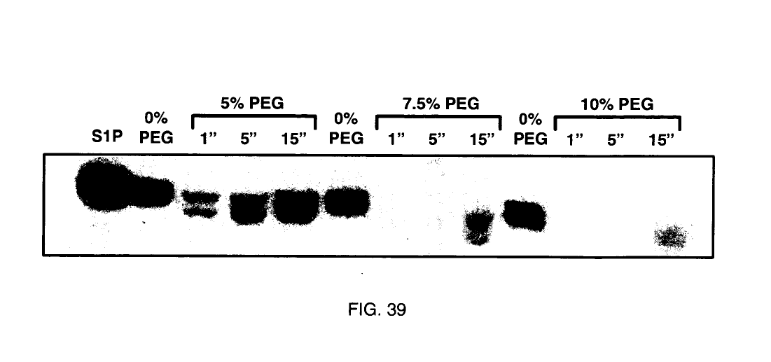

FIG. 39 shows data establishing that HMW PEG inhibited ERK and MLC phosphorylation. S1P, a barrier-enhancing agent, has been shown to result in activation of specific signal transduction pathways, as detected by phosphorylation of ERK and MLC. Thus, the effects of HMW PEG, at various concentrations and treatment times, on ERK and MLC phosphorylation were examined by Western blot analyses. In contrast to S1P, HMW PEG potently inhibited basal phosphorylation of ERK (see FIG. 39) and MLC.

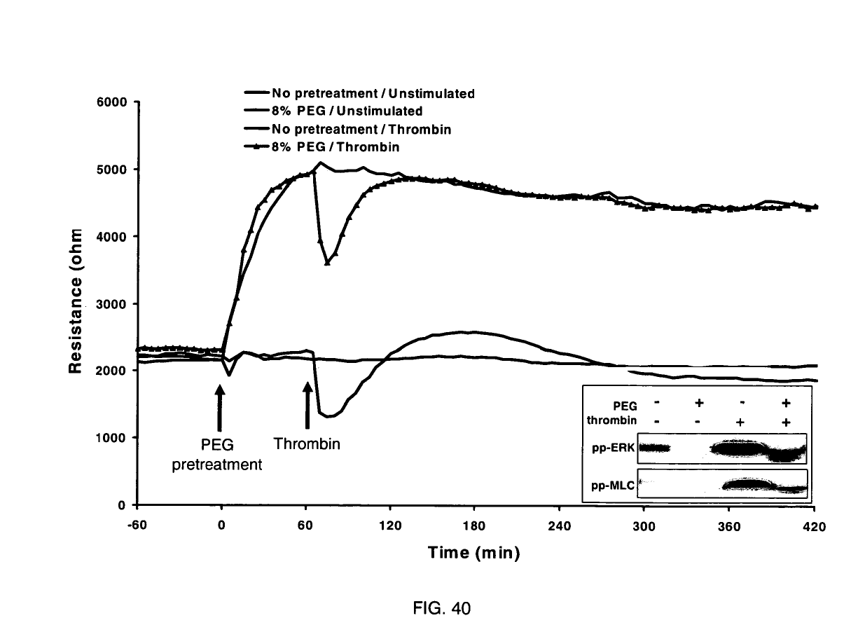

In FIG. 40, data are presented that show that HMW PEG pretreatment protects cells from thrombin-induced barrier disruption. Cells were pretreated with HMW PEG (8%>, 1 hour) and subsequently challenged with thrombin (1 U/ml). HMW PEG induced a rapid and sustained barrier enhancement, which was able to provide barrier protection from thrombin by preventing a decrease in resistance below basal level.

These experiments also established that HMW PEG blocked thrombin-induced ERK phosphorylation and attenuated MLC phosphorylation. Because HMW PEG inhibited basal ERK and MLC phosphorylation, the ability of HMW PEG to inhibit thrombin-induced ERK and MLC activation was examined. Cells were treated with HMW7 PEG and challenged with thrombin (1 U/ml, 5 minutes). Lysates were processed for Western blot analyses and the resulting Western blots were probed with phospho-specific antibodies to ERK and MLC. HMW PEG completely blocked thrombin-induced ERK phosphorylation and potently attenuated thrombin-induced MLC phosphorylation.

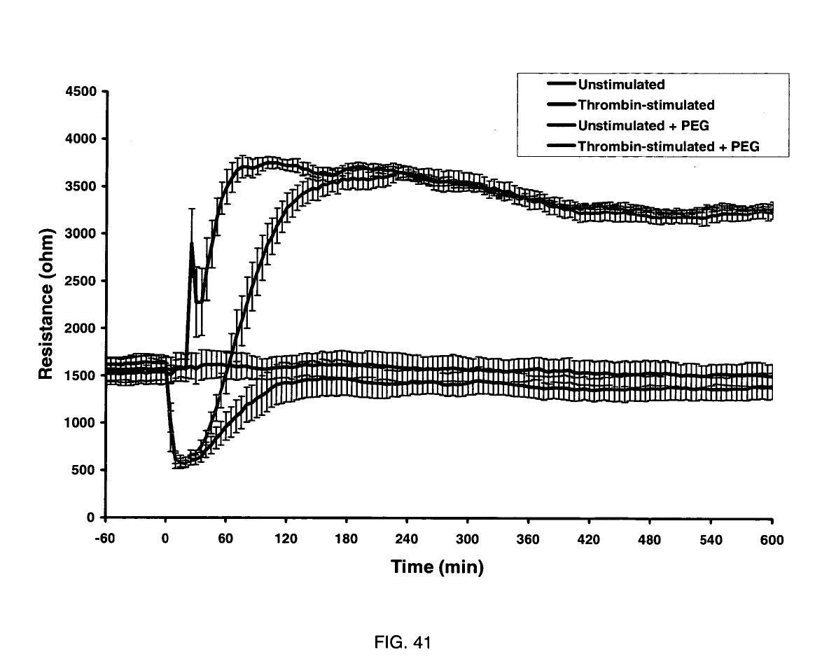

Further, the data in FIG. 41 show that HMW PEG potently reversed thrombin-induced barrier disruption. Reversing paracellular barrier disruption is clinically more relevant, in general, than prevention of barrier disruption. Thus, the ability of HMW PEG to reverse and enhance endothelial banner function after thrombin challenge was examined. HPAEC were stimulated with thrombin (1 U/ml) for 20 minutes and subsequently treated with HMW PEG (8%) and compared with unstimulated cells or cells without thrombin stimulation. Thrombin induced a rapid and dynamic decrease in banier resistance. Addition of HMW PEG at the maximum thrombin-induced decrease in resistance resulted in a rapid barrier reversal and increased barrier resistance to levels observed with HMW PEG-treatment alone (i.e., exposure of cells to HMW PEG without any exposure to thrombin). Thus, HMW PEG reversed thrombin-induced barrier disruption and induced sustained banier integrity after thrombin insult.

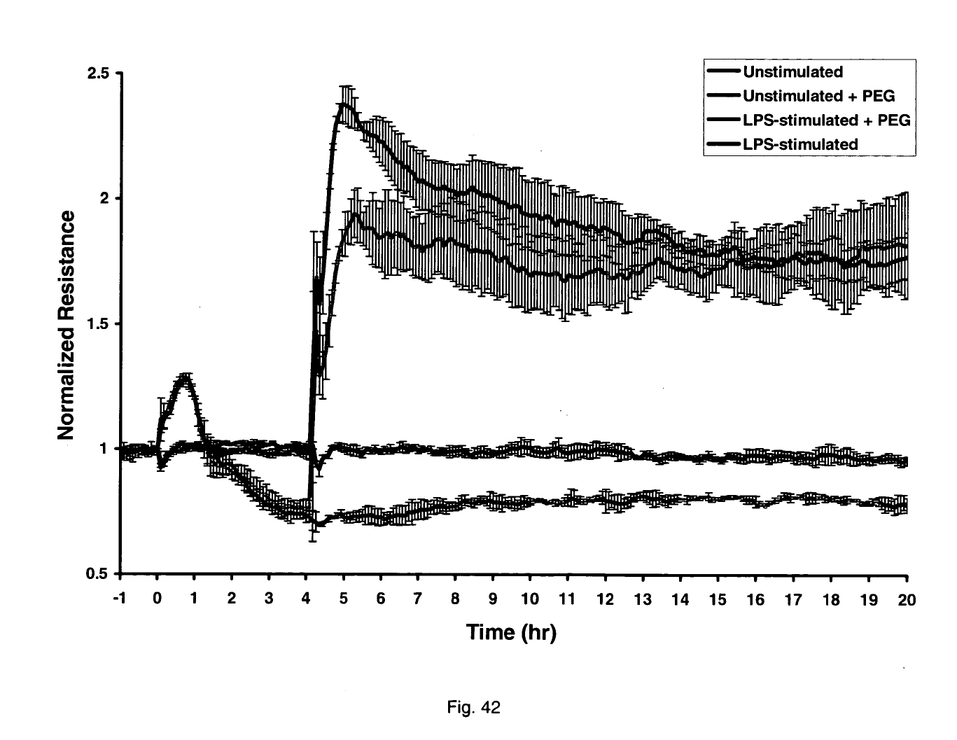

HMW PEG also potently reversed LPS-induced banier disruption, as shown by the data in FIG. 42. Because thrombin is a rapid and temporal edemagenic agent, the effect of HMW PEG on cells exposed to a gradual barrier-inducing agent, LPS, was examined. HPAEC were stimulated with LPS (5 U/ml) for 4 hours and were subsequently treated with HMW PEG (8%) and compared with unstimulated cells or cells without LPS stimulation. LPS induced a gradual and prolonged decrease in barrier resistance for over 20 hours. Addition of HMW PEG after 4 hours of LPS challenge resulted in a rapid banner reversal and increased barrier resistance to levels near the cell barrier resistance resulting from HMW PEG-treatment alone (i.e., no exposure to LPS). Similar to thrombin challenge, HMW PEG also reversed LPS-induced barrier disruption and induced sustained barrier integrity after LPS insult.

In summary, ECs were cultured on microelectrodes and changes in transendothelial electrical resistances (TEER) were measured to assess alterations in paracellular permeability. HMW PEG induced a rapid, dose-dependent increase in TEER. An optimal concentration of 8% HMW PEG induced a robust increase in TEER from 2500 to 5000 ohm, which was sustained for 40 hours. Although 8% HMW PEG was optimal in the experiments described herein, other concentrations of HMW PEG are contemplated, with preferred but non-exclusive ranges of 5-10% and 5-8% HMW PEG being noted. Brief, 30-minute exposure to HMW PEG resulted in a rapid increase in resistance that quickly returned to near baseline levels upon HMW PEG removal, followed by a gradual return to near peak levels, suggesting potential gene upregulation. The diminished HMW PEG removal response was restored by addition of sphingosine 1-phosphate to a level above addition of the lipid alone, suggesting a rapid mechanism mediated by signal transduction. HMW PEG rapidly induced dephosphorylation of ERK and MLC as early as one minute post-exposure, and completely inhibited thrombin-induced ERK and MLC phosphorylations.

Immunofluorescence data revealed that HMW PEG altered the EC actin cytoskeleton to form a defined cortical actin ring that is expected to help strengthen cell-cell junctional adhesion. Thus, HMW PEG activated a rapid, actin-associated, barrier-enhancing signal transduction pathway in EC, which is expected to have therapeutic application in the prevention or treatment of lung disorders such as pulmonary edema, including reversal of the progression of the disorder, as well as application in the mitigation of at least one symptom associated with lung disorders such as pulmonary edema. The results disclosed in this Example and elsewhere in this application establish the protective effects of HMW PEG to bacterial infection, notably including microbe-induced sepsis, such as lung injury-associated sepsis, as well as disorders of the gastrointestinal tract and, more generally epithelial and endothelial cell disorders, such as inflammatory disorders.

The references cited throughout this disclosure are hereby incorporated by reference in their entireties.

1. A method of treating a disorder of a lung cell comprising administering to an organism in need thereof a therapeutically effective amount of a high molecular weight polyethylene glycol-like compound having an average molecular weight of at least 8,000 daltons.

2. The method according to claim 2 wherein the polyethylene glycol has an average molecular weight of at least 15,000 daltons.

3. The method according to claim 2 wherein the lung cell is selected from the group consisting of an endothelial cell and an epithelial cell.

4. The method according to claim 3 wherein the lung cell is an endothelial cell.

5. The method according to claim 4 wherein the disorder is selected from the group consisting of acute respiratory distress syndrome and acute lung injury.

6. The method according to claim 2 wherein the polyethylene glycol-like compound is selected from the group consisting of straight-chain polyethylene glycol, branched-chain polyethylene glycol and polyethylene glycol comprising an aromatic functional group.

7. The method according to claim 6 wherein the aromatic functional group is an unsubstituted or substituted phenol group.

8. The method according to claim 1 wherein the therapeutically effective amount is an amount sufficient to detectably inhibit ERK phosphorylation or MLC phosphorylation in a lung cell of the organism being treated.

9. The method according to claim 1 wherein the organism is a human.

10. A method of ameliorating a symptom associated with a lung cell disorder comprising administering to an organism in need thereof a therapeutically effective amount of a high molecular weight polyethylene glycol-like compound having an average molecular weight selected from the group consisting of at least 8,000 daltons and at least 15,000 daltons.

11. The method according to claim 10 wherein the lung cell is an endothelial cell.

12. The method according to claim 11 wherein the disorder is selected from the group consisting of acute respiratory distress syndrome and acute lung injury.

13. The method according to claim 10 wherein the organism is a human.

14. A method of preventing a lung cell disorder comprising administering to an organism at risk a prophylactically effective amount of a high molecular weight polyethylene glycol-like compound having an average molecular weight selected from the group consisting of at least 8,000 daltons and at least 15,000 daltons.

15. The method according to claim 14 wherein the lung cell is an endothelial cell.

16. The method according to claim 14 wherein the disorder is selected from the group consisting of acute respiratory distress syndrome and acute lung injury.

17. The method according to claim 14 wherein the prophylactically effective amount is an amount sufficient to detectably inhibit ERK phosphorylation or MLC phosphorylation in a lung cell of the organism at risk.

18. The method according to claim 14 wherein the organism is a human.

19. A kit comprising a high molecular weight polyethylene glycol (HMW PEG) according to claim 6 and a set of instructions for administering the HMW PEG to treat, prevent or ameliorate a lung disorder.

20. The kit according to claim 19 wherein the HMW PEG has a molecular weight of at least 15,000 daltons.

21. A method of preventing, inhibiting, or reversing a paracellular barrier disruption comprising administration of a therapeutically effective amount of a high molecular weight polyethylene glycol according to claim 6.

22. The method according to claim 21 wherein the paracellular barrier disruption is produced by an edemagenic agent selected from the group consisting of thrombin and lipopolysaccharide.

23. The method according to claim 21 further comprising enhancement of a cortical actin cytoskeleton.

24. The method according to claim 21 wherein said paracellular barrier disruption is an endothelial cell barrier disruption.

25. The method according to claim 24 wherein said high molecular weight polyethylene glycol inhibits phosphorylation of a protein selected from the group consisting of ERK and MLC in said cell.
